(19) United States
(12) Patent Application Publication
(10) Pub. No.: US 2003/0021792 A1
(43) Pub. Date: Jan. 30, 2003

(54) Tissue-specific endothelial membrane proteins

(76) Inventors: Paul W. Roben, San Diego, CA (US); Anthony C. Stevens, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/165,603

(22) Filed: Jun. 7, 2002

Related U.S. Application Data
(60) Provisional application No. 60/297,021, filed on Jun. 8, 2001.
(60) Provisional application No. 60/305,117, filed on Jul. 12, 2001.

(51) Int. Cl. A61K 39/395; A61K 38/43; A61K 9/127; C07K 16/46

(52) U.S. Cl. 424/178.1; 530/391.1; 424/450; 424/94.1

(57) ABSTRACT

Methods and compositions for targeting of pharmaceuticals or other therapeutics to specific tissues using tissue-specific endothelial membrane proteins are provided. The compositions comprise a therapeutic complex composed of a ligand, a linker, and a therapeutic moiety, where the therapeutic moiety can enter the cell. The ligand can be an antibody or other molecule that binds to a tissue-specific protein on the endothelial membrane of a specific tissue. The ligand need not activate a receptor, but may activate endocytosis. The therapeutic moiety can be a drug, gene, antisense oligonucleotide, contrast agent, protein, toxin, or any type of molecule that acts on the specific tissue. The linker can be a liposome or a cleavable or noncleavable chemical molecule. Alternatively, the linker may simply be the bond between the ligand and the therapeutic moiety. Alternatively, a lipophilic prodrug may be cleaved and may enter the cell due to its lipophilic properties.

### RELATED APPLICATIONS

This application claims priority to U.S. Provisional Patent Application Serial No. 60/297,021, filed Jun. 8, 2001, by Paul Roben, et al., and entitled “TISSUE-SPECIFIC ENDOTHELIAL MEMBRANE PROTEINS” and U.S. Provisional Patent Application Serial No. 60/305,117, filed Jul. 12, 2001, by Paul Roben, et al., and entitled “TISSUE-SPECIFIC ENDOTHELIAL MEMBRANE PROTEINS”, the disclosures of which are incorporated herein by reference in their entirety.

### BACKGROUND OF THE INVENTION

1. Field of the Invention

This invention relates generally to targeting of pharmaceuticals or other therapeutics to specific tissues using tissue specific endothelial membrane proteins.

2. Description of the Related Art

When conventional pharmaceuticals are delivered to a patient they circulate throughout the entire body of the patient and act on most if not all tissues or cells of the body. This requires high doses for treatment and results in systemic toxicity and side effects.

Targeted delivery of therapeutic or diagnostic agents to specific organs, tissues or cells is much safer and more effective then such a non-specific treatment, because much smaller amounts of the drug are needed and there is considerably less chance for side-effects or toxicity.

Previous methods for the targeted delivery of pharmaceuticals include the use of implants (e.g., Elise (1999) PNAS USA 96:3104-3107), stents or catheters (e.g., Murphy (1992) Circulation 86:1596-1604), or vascular isolation of an organ (e.g., Vahrmeijer (1998) Semin. Surg. Oncol. 14:262-268). However, these techniques are invasive, traumatic and can cause extensive inflammatory responses and fibrocellular proliferation.

Most previous attempts at tissue-specific delivery depended on sites within the tissue that were inaccessible to the compounds due to the natural barrier of the vasculature. An alternative method for targeted delivery of compounds involves organ or tissue-specific molecules exposed on the luminal surface of the vasculature rather than on the tissue cells themselves. Use of these molecules would allow for a very specific reaction. The specificity is due to the fact that blood vessels must express these tissue-specific endothelial proteins because the vasculature forms a complex and dynamic system which adapts to the needs of the tissue in which it is immersed.

Previously, methods for identifying these organ or tissue-specific molecules, which were exposed and accessible on the luminal surface of the vasculature, did not result in the identification of usable molecules. This is because the endothelial membrane represents only a miniscule portion of the tissue mass of any organ. When organs are analyzed by conventional means, the endothelial membranes become dispersed throughout the entire tissue homogenate. This renders isolation of the endothelial membrane and its proteins for separate analysis essentially impossible. In addition, even if isolated and in culture, these membranes tend to lose their tissue-specific properties. In the event such molecules are isolated in a useful manner, methods must be conceived which allow for uses of these molecules related to the treatment of diseases in patients.

### SUMMARY OF THE INVENTION

There are several exemplary embodiments of the instant invention. One such embodiment includes a method for delivering a therapeutic agent to a specific tissue, comprising: administering a therapeutically effective amount of a therapeutic complex, said therapeutic complex comprising: a ligand which binds to a tissue-specific luminally expressed protein, a therapeutic moiety, and a linker which links said therapeutic moiety to said ligand.

Another embodiment includes a lung and/or heart-specific therapeutic complex which interacts with a targeted endothelial cell, comprising: a ligand which attaches said therapeutic complex to the luminal surface of a vascular endothelial cell membrane of the specific tissue, wherein said ligand binds to SEQ ID NO:9 or 11, or a homolog thereof; a linker; and a therapeutic moiety, wherein said linker links the ligand to the therapeutic moiety.

Another embodiment includes a method of determining the presence or concentration of Carbonic anhydrase IV (CA-4) in a tissue or cell, comprising administering the above lung and/or heart-specific therapeutic complex to said tissue or cell in vitro or in vivo, and identifying or quantitating the amount of the therapeutic complex which bound.

Another embodiment includes a pharmaceutical composition comprising the above lung and/or heart-specific therapeutic complex and one or more pharmaceutically acceptable carriers.

Another embodiment includes a lung and/or kidney-specific therapeutic complex which interacts with a targeted endothelial cell, comprising: a ligand which attaches said therapeutic complex to the luminal surface of a vascular endothelial cell membrane of the specific tissue, wherein the ligand binds to SEQ ID NO:4 or 6, or a homolog thereof; a linker; and a therapeutic moiety, wherein said linker links the ligand with the therapeutic moiety.

Another embodiment includes a method of determining the presence or concentration of dipeptidyl peptidase IV (DPP-4) in a tissue or cell, comprising administering the above lung and/or kidney-specific therapeutic complex to said tissue or cell in vitro or in vivo, and identifying or quantitating the amount of the therapeutic complex which bound.

Another embodiment includes a pharmaceutical composition comprising the above lung and/or kidney-specific therapeutic complex and one or more pharmaceutically acceptable carriers.

Another embodiment includes a pancreatic and/or gut-specific therapeutic complex which interacts with a targeted endothelial cell, comprising: a ligand which attaches said therapeutic complex to the luminal surface of a vascular endothelial cell membrane of the specific tissue, wherein said ligand binds to SEQ ID NO:14 or 16, or a homolog thereof; a linker; and a therapeutic moiety, wherein said linker links the ligand with the therapeutic moiety.

Another embodiment includes a method of determining the presence or concentration of ZG16-p in a tissue or cell, comprising administering the above pancreatic and/or gut-specific therapeutic complex to said tissue or cell in vitro or in vivo, and identifying or quantitating the amount of the therapeutic complex which bound.

Another embodiment includes a pharmaceutical composition comprising the pancreatic and/or gut-specific therapeutic complex and one or more pharmaceutically acceptable carriers.

Another embodiment includes a prostate-specific therapeutic complex which interacts with a targeted endothelial cell, comprising: a ligand which attaches said therapeutic complex to the luminal surface of a vascular endothelial cell membrane of the specific tissue comprising SEQ ID NO:23 or a homolog thereof; a linker; and a therapeutic moiety, wherein said linker links the ligand with the therapeutic moiety.

Another embodiment includes a method of determining the presence or concentration of Albumin fragment in a tissue or cell, comprising administering the above prostate-specific therapeutic complex to said tissue or cell in vitro or in vivo, and identifying or quantitating the amount of the therapeutic complex which bound.

Another embodiment includes a pharmaceutical composition comprising the prostate-specific therapeutic complex and one or more pharmaceutically acceptable carriers.

Another embodiment includes a brain-specific therapeutic complex which interacts with a targeted endothelial cell, comprising: a ligand which attaches said therapeutic complex to the luminal surface of a vascular endothelial cell membrane of the specific tissue wherein said ligand binds to SEQ ID NO:26 or 28 or a homolog thereof; a linker; and a therapeutic moiety, wherein said linker links the ligand with the therapeutic moiety.

Another embodiment includes a method of determining the presence or concentration of CD71 (transferrin receptor) in a tissue or cell, comprising administering the above brain-specific therapeutic complex to said tissue or cell in vitro or in vivo, and identifying or quantitating the amount of the therapeutic complex which bound.

Another embodiment includes a pharmaceutical composition comprising the above brain-specific therapeutic complex and one or more pharmaceutically acceptable carriers.

Another embodiment includes a pancreas and/or gut-specific therapeutic complex which interacts with a targeted endothelial cell, comprising: a ligand which attaches said therapeutic complex to the luminal surface of a vascular endothelial cell membrane of the specific tissue wherein said ligand binds to SEQ ID NO:18 or 20, or a homolog thereof; a linker; and a therapeutic moiety, wherein said linker links the ligand with the therapeutic moiety.

Another embodiment includes a method of determining the presence or concentration of MAdCAM (MadCam-1) in a tissue or cell, comprising administering the above pancreas and/or gut-specific therapeutic complex to said tissue or cell in vitro or in vivo, and identifying or quantitating the amount of the therapeutic complex which bound.

Another embodiment includes a pharmaceutical composition comprising the above pancreas and/or gut-specific therapeutic complex and one or more pharmaceutically acceptable carriers.

Another embodiment includes a kidney-specific therapeutic complex which interacts with a targeted endothelial cell, comprising: a ligand which attaches said therapeutic complex to the luminal surface of a vascular endothelial cell membrane of the specific tissue, wherein said ligand binds to SEQ ID NO:30 or 32, or a homolog thereof; a linker; and a therapeutic moiety, wherein said linker links the ligand to the therapeutic moiety.

Another embodiment includes a method of determining the presence or concentration of CD90 in a tissue or cell, comprising administering the above kidney-specific therapeutic complex to said tissue or cell in vitro or in vivo, and identifying or quantitating the amount of the therapeutic complex which bound.

Another embodiment includes a pharmaceutical composition comprising the above kidney-specific therapeutic complex and one or more pharmaceutically acceptable carriers.

Another embodiment includes a method for the treatment of prostate cancer comprising administering the above prostate-specific therapeutic complex in an amount effective to reduce the number of cancer cells, wherein said therapeutic moiety is a chemotherapeutic agent.

Another embodiment includes a method for the treatment of brain tumors comprising administering the above brain-specific therapeutic complex in an amount effective to reduce the number of cancer cells, wherein said therapeutic moiety is a chemotherapeutic agent.

Another embodiment includes a method for the treatment of pancreatic cancer comprising administering one or more of the above pancreas and/or gut-specific therapeutic complexes in an amount effective to reduce the amount of thrombosis, wherein said therapeutic moiety is an antithrombotic agent.

Another embodiment includes a method for the treatment of kidney transplant rejection comprising administering the above lung and/or kidney specific therapeutic complex in an amount sufficient to reduce the rejection of the kidney transplant, wherein said therapeutic moiety is an immunosuppressant agent.

Another embodiment includes a method for delivering a therapeutic agent to a specific tissue, comprising: administering a therapeutically effective amount of a therapeutic complex, said therapeutic complex comprising: a ligand which binds to a tissue-specific luminally expressed protein, a therapeutic moiety, and a linker which links said therapeutic moiety to said ligand, wherein said tissue-specific luminally expressed protein is selected from the group consisting of CD71, CD90, MAdCAM, Albumin fragment, carbonic anhydrase IV, ZG16-p and dipeptidyl peptidase IV.

Another embodiment includes a method for lung and/or heart-specific delivery of a substance in vivo or in vitro, comprising: providing a carbonic anhydrase IV-binding agent, and administering said carbonic anhydrase IV-binding agent in vivo or in vitro, wherein said substance is delivered to the lung and/or heart or lung and/or heart tissue as a result of the administration of the carbonic anhydrase IV-binding agent.

Another embodiment includes a method of identifying a lung and/or heart-specific ligand, comprising identifying a carbonic anhydrase IV-binding agent.

Another embodiment includes a method for brain-specific delivery of a substance in vivo or in vitro, comprising: providing a CD71 (transferrin receptor)-binding agent, and administering said CD71-binding agent in vivo or in vitro, wherein said substance is delivered to the brain or brain tissue as a result of the administration of the CD71-binding agent.

Another embodiment includes a method of identifying a brain-specific ligand, comprising identifying a CD71-binding agent.

Another embodiment includes a method for kidney-specific delivery of a substance in vivo or in vitro, comprising: providing a CD90 (Thy-1)-binding agent, and administering said CD90-binding agent in vivo or in vitro, wherein said substance is delivered to the kidney or kidney tissue as a result of the administration of the CD90-binding agent.

Another embodiment includes a method of identifying a kidney-specific ligand, comprising identifying a CD90-binding agent.

Another embodiment includes a method for lung and/or kidney-specific delivery of a substance in vivo or in vitro, comprising: providing a dipeptidyl peptidase IV-binding agent, and administering said dipeptidyl peptidase IV-binding agent in vivo or in vitro, wherein said substance is delivered to the lung and/or kidney or lung and/or kidney tissue as a result of the administration of the dipeptidyl peptidase IV-binding agent.

Another embodiment includes a method of identifying a lung and/or kidney-specific ligand, comprising identifying a dipeptidyl peptidase IV-binding agent.

Another embodiment includes a method for pancreas and/or gut-specific delivery of a substance in vivo or in vitro, comprising: providing a ZG16-p-binding agent, and administering said ZG16-p-binding agent in vivo or in vitro, wherein said substance is delivered to the pancreas and/or gut or pancreas and/or gut tissue as a result of the administration of the ZG16-p-binding agent.

Another embodiment includes a method of identifying a pancreas and/or gut-specific ligand, comprising identifying a ZG16-p-binding agent.

Another embodiment includes a method for pancreas and/or gut-specific delivery of a substance in vivo or in vitro, comprising: providing a MAdCAM-binding agent, and administering said MAdCAM-binding agent in vivo or in vitro, wherein said substance is delivered to the pancreas and/or gut or pancreas and/or gut tissue as a result of the administration of the MAdCAM-binding agent.

Another embodiment includes a method of identifying a pancreas and/or gut-specific ligand, comprising identifying a MAdCAM-binding agent.

Another embodiment includes a method for prostate-specific delivery of a substance in vivo or in vitro, comprising: providing a Albumin fragment-binding agent, and administering said Albumin fragment-binding agent in vivo or in vitro, wherein said substance is delivered to the prostate or prostate tissue as a result of the administration of the Albumin fragment-binding agent.

Another embodiment includes a method of identifying a prostate-specific ligand, comprising identifying an Albumin fragment-binding agent.

### BRIEF DESCRIPTION OF THE DRAWINGS

FIG. 1 is a depiction of a typical therapeutic complex interacting with an endothelial cell surface, tissue-specific molecule.

FIGS. 2A-D show the immunohistochemistry of tissue sections from a rat which was injected with either CD71 or a control antibody. FIG. 2A is Brain from a rat injected with CD71, FIG. 2B is Brain from a rat injected with the control antibody, FIG. 2C is lung from a rat injected with CD71, FIG. 2D is lung from a rat injected with the control antibody.

FIG. 3 shows a polyacrylamide gel of luminal proteins isolated from lung. Dipeptidyl peptidase IV is labeled DPP-4.

FIGS. 4A-F are a series of immunohistograms of various tissues showing binding of an anti-dipeptidyl peptidase antibody to luminal tissue in kidney and lung.

FIG. 5 shows a polyacrylamide gel of another set of luminal proteins isolated from lung. Carbonic Anhydrase IV is labeled CA-4.

FIG. 6 shows a polyacrylamide gel of luminal proteins isolated from pancreas. Zymogen granule 16 protein is labeled ZG16P.

FIGS. 7A-F are a series of immunohistograms of various tissues showing binding of a MAdCAM antibody to luminal tissue in pancreas and colon.

FIGS. 8A-F are a series of immunohistograms of various tissues showing binding of a Thy-1 (CD90) antibody to luminal tissue in the kidney.

FIG. 9 shows a polyacrylamide gel of luminal proteins isolated from prostate. The albumin fragment is labeled T406-608.

FIGS. 10A-D are a series of immunohistograms of various tissues showing binding of OX-61 to dipeptidyl peptidase IV, which is expressed on the luminal surface of the vasculature of the lung.

FIGS. 11A-D are a series of immunohistograms of various tissues showing binding of OST-2 to MadCam-1, which is expressed on the luminal surface of the vasculature of the pancreas and colon.

FIGS. 12A-F are a series of immunohistograms of various tissues showing binding of OX-7 to CD90, which is expressed on the luminal surface of the vasculature of the kidney.

FIGS. 13A-F are a series of immunohistograms of various tissues showing binding of an anti-carbonic anhydrase IV antibody to carbonic anhydrase IV, which is expressed on the luminal surface of the vasculature of the heart and lung.

FIGS. 14A-E are a series of immunohistograms of lung showing a profile of the binding of OX-61 to dipeptidyl peptidase IV over a twenty-four hour timecourse.

FIGS. 15A-D are a series of immunohistograms of pancreas showing a profile of the binding of OST-2 to MadCam-1 over a forty-eight hour timecourse.

FIGS. 16A-F are a series of immunohistograms of kidney showing a profile of the binding of OX-7 to CD90 over an eight hour timecourse.

FIGS. 17A-C are graphs which show the fraction of the injected dose of Europium-labeled OX-61 that localized to lung over a twenty-four hour time period. The dashed line indicates the maximum level of isotype control antibody that bound to any of the indicated tissues at any time point.

FIGS. 18A-C are graphs which show the fraction of the injected dose of Europium-labeled anti-influenza IgG2A isotype control antibody that localized to specific tissues over a twenty-four hour time period.

FIGS. 19A-C are graphs which show the fraction of the injected dose of Europium-labeled OST-2 that localized to pancreas over a twenty-four hour time period. The dashed line indicates the maximum level of isotype control antibody that bound to any of the indicated tissues at any time point.

FIG. 20 is a graph which shows the fraction of the injected dose of Europium-labeled anti-carbonic anhydrase W antibody that localized to heart and lung over a twenty-four hour time period.

FIG. 21 is a graph which shows the amount of injected ¹²⁵I-labeled OX-61 that localized to various tissues and fluids over an eight hour time period.

FIG. 22 is an immunohistogram of a section of lung which shows the transcytotic transport of OX-61 by dipeptidyl peptidase IV.

FIG. 23 is an immunohistogram of a section of kidney which shows the transcytotic transport of OX-7 by CD90.

FIG. 24 is an immunohistogram of a section of pancreas which shows that OST-2 binds to MadCam-1 on the luminal surface of the vasculature but is not transported across the endothelium.

FIG. 25 is an immunohistogram of a section of lung which shows that anti-carbonic anhydrase IV antibody binds to carbonic anhydrase IV on the luminal surface of the vasculature but is not transported across the endothelium.

FIGS. 26A-F are a series of immunohistograms of various tissues showing binding of an OX-61/gentamicin therapeutic complex to dipeptidyl peptidase IV, which is expressed on the luminal surface of the vasculature of the lung.

FIGS. 27A-D are a series of immunohistograms of various tissues showing binding of an OX-61/doxorubicin therapeutic complex to dipeptidyl peptidase IV, which is expressed on the luminal surface of the vasculature of the lung.

FIG. 28 is an immunohistogram of a section of lung which shows the transcytotic transport of an OX-61/gentamicin therapeutic complex by dipeptidyl peptidase IV.

FIG. 29 is an immunohistogram of a section of lung which shows the transcytotic transport of an OX-61/doxorubicin therapeutic complex by dipeptidyl peptidase IV.

FIGS. 30A-F are a series of immunohistograms of various tissues showing binding of an OST-2/gentamicin therapeutic complex to MadCam-1, which is expressed on the luminal surface of the vasculature of the colon and pancreas.

FIGS. 31A-F are a series of immunohistograms of various tissues showing binding of an OST-2/doxorubicin therapeutic complex to MadCam-1, which is expressed on the luminal surface of the vasculature of the colon and pancreas.

FIGS. 32A-B are graphs which show the amount of free gentamicin that accumulated in the lung and the kidney over an eighteen hour time period compared to the amount that was delivered to these tissue in DSPC-DPP therapeutic complexes.

FIGS. 33A-B are graphs which show the amount of free gentamicin that accumulated in various tissues over an eighteen hour time period compared to the amount that was delivered to these tissue in EPC-DPP therapeutic complexes and untargeted liposomes.

FIGS. 34A-B are graphs which show the amount of free gentamicin that accumulated in various tissues over an eighteen hour time period compared to the amount that was delivered to these tissue in DSPC-DPP therapeutic complexes and untargeted liposomes.

FIG. 35 is a graph which shows the efficacy of both free gentamicin and gentamicin in EPC-DPP therapeutic complexes in the treatment of lung infections.

### DETAILED DESCRIPTION OF THE PREFERRED EMBODIMENT

One embodiment described herein supplies both compositions and methods of use of therapeutic compounds for delivery to a specific tissue whether or not such tissue is in a diseased state. Specifically, the invention utilizes tissue-specific luminally exposed proteins on endothelial cells so that the tissue-specific therapeutic complexes described herein will localize to a specific tissue due to binding of these complexes to luminally-exposed endothelial proteins. This embodiment allows for localization and concentration of a pharmaceutical agent to a specific tissue, thus increasing the therapeutic index of that pharmaceutical agent. This localization decreases the chances of side effects due to the agent and may allow one to use a lower concentration of the agent to achieve the same effect. Localization to a luminally-exposed tissue specific endothelial protein affords the added advantage that a single ligand can be used to treat a variety of diseases involving that tissue. In other words, a disease specific ligand for each disease state of a tissue need not be generated; as sufficient amounts of one or more therapeutic complexes will bind to the effected tissue which is expressing a protein normally found on the luminal endothelial cells of that tissue or organ. This feature allows the use of a single ligand to produce therapeutic complexes to treat any disease associated with the tissue. The tissue-specific molecule may be identified by the method of U.S. patent application Ser. No. 09/528,742, filed Mar. 20, 2000, herein incorporated by reference, or any other method of identification. The method disclosed in U.S. patent application Ser. No. 09/528,742 permits the in vivo isolation of all proteins that are exposed on the inner surface of blood vessels from different tissues. All other proteins that make up the tissues (which are the vast majority) are discarded in the process. The resulting set of luminally exposed vascular proteins can then be separated and analyzed biochemically to identify each protein individually. By comparing the set of proteins expressed in each tissue, proteins are identified that are specific to a given tissue. Proteins of interest are then sequenced. Ligands are obtained that specifically bind to the target protein. These ligands, upon binding to the target protein, or the protein that is tissue-specifically luminally expressed, preferably does not activate a specific signal transduction pathway in the cell it binds to, but may activate the process of transcytosis or pinocytosis.

Endothelial cell tissue-specific proteins are accessible to the blood, and thus, they can act at site-specific targets used to localize therapeutic complexes to a specific tissue. Blood vessels express these tissue-specific endothelial proteins because the vasculature forms a complex and dynamic system which adapts to the needs of the tissue in which it is immersed. Many of these proteins are constitutively expressed, meaning that their levels of expression are not significantly changed in different disease states, making them ideal targets for the delivery of pharmaceuticals whether or not the tissue or organ containing the tissue is in the diseased state. In addition, many of these proteins are involved in transcytosis, the process of transporting materials from within the blood vessels into the tissue.

### Definitions

Unless defined otherwise, all technical and scientific terms used herein have the meaning commonly understood by a person of skill in the art to which this invention belongs. As used herein, the following terms have the meanings ascribed to them unless specified otherwise.

As used herein, the term “gut” is synonymous with gastrointestinal (GI) tract.

The term “target protein” as used herein is a tissue-specific, luminally exposed vascular protein.

The term “ligand” as used herein is a molecule that specifically binds to the target protein. These can be peptides, antibodies or parts of antibodies, as well as non-protein moieties.

The term “linker” as used herein is any bond, small molecule, or other vehicle which allows the ligand and the therapeutic moiety to be targeted to the same area, tissue, or cell. The linker binds or otherwise holds together the ligand and the therapeutic moiety for binding to the target protein.

The term “therapeutic moiety” as used herein is any type of substance which can be used to effect a certain outcome. The outcome can be positive or negative, alternatively, the outcome can simply be diagnostic. The outcome may also be more subtle such as simply changing the molecular expression in a cell. The therapeutic moiety may also be an enzyme which allows conversion of a prodrug into the corresponding pharmaceutical agent.

The term “therapeutic complex” is any type of molecule which includes a ligand specific for a target protein and one or more therapeutic moieties and a linker. However, it is to be understood that a therapeutic complex may also comprise an enzyme or some other inducer of cleavage which allows a prodrug to be converted into the corresponding pharmaceutical agent.

The term “tissue-specific” refers to a molecule that is preferentially expressed on a specific tissue or cell-type, allowing a substantial fraction of the therapeutic complex to bind to that tissue after administration. The molecule may be found at a considerably higher concentration in one or a few tissues than in the others. For example, a tissue-specific molecule may be highly upregulated in the lung compared to other tissues but can be dosed to be even more specific based on the statistical distribution of binding throughout the vasculature. Proper, often lower, dosing of the therapeutic complex would be given such that the amounts that appear randomly at non-targeted tissue would render little or no side effects.

### General Techniques

The embodiment described herein can be practiced in conjunction with any method or protocol known in the art and described in the scientific and patent literature. The various compositions (e.g., natural or synthetic compounds, polypeptides, peptides, nucleic acids, antibodies, toxins, and the like) used in the embodiment described herein can be isolated from a variety of sources, genetically engineered, amplified, and/or expressed recombinantly. Alternatively, these compositions can be synthesized in vitro by well-known chemical synthesis techniques, as described in, e.g., Organic Synthesis, collective volumes, Gilman et al. (Eds) John Wiley & Sons, Inc., NY; Carruthers (1982) Cold Spring Harbor Symp. Quant. Biol. 47:411-418; and Caruthers et al, U.S. Pat. No. 4,458,066, Jul. 3, 1984.

### Therapeutic Complexes

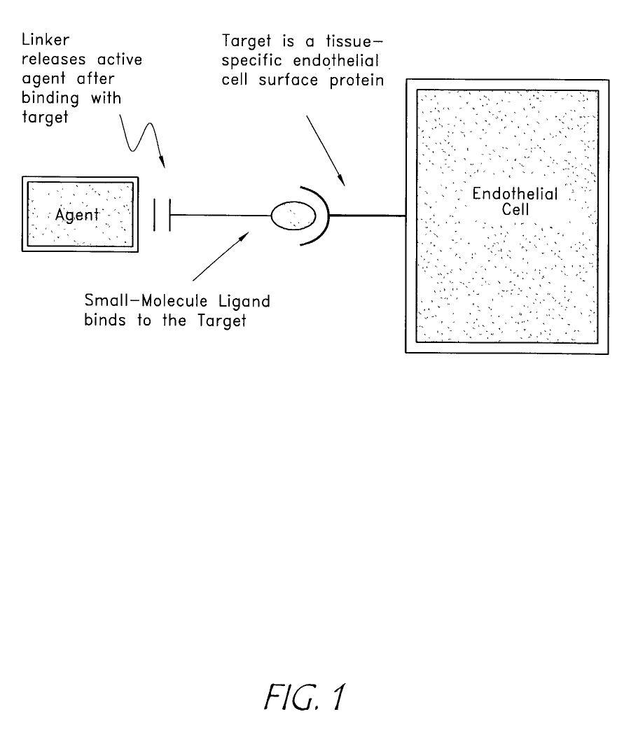

The therapeutic complexes of the invention bind to the target proteins, for example from the pancreas, lung, muscle, intestine, prostate, kidney, and brain to specifically deliver a therapeutic moiety to the tissue or organ of choice. The therapeutic complexes are composed of at least one ligand, a linker, and at least one therapeutic moiety (see FIG. 1). However, the attachment of the three types of components of the therapeutic complex can be envisioned to have a large number of different embodiments. The therapeutic moiety can be one or more of any type of molecule which is used in a therapeutic or diagnostic way. For example, the therapeutic moiety can be an antibiotic which needs to be taken up by a specific tissue. The therapeutic complex can be envisioned to concentrate and target the antibiotic to the tissue where it is needed, thus increasing the therapeutic index of that antibiotic. Alternatively, the therapeutic moiety can be for in vivo or in vitro diagnostic purposes. Further examples of the use of therapeutic complexes in the specific embodiments of the present invention will be outlined in more detail in the section entitled “Type of Therapeutic Complex Interactions”.

### Ligands

The ligand is a molecule which specifically binds to the target protein, in this case, the luminally-expressed tissue-specific proteins. In one embodiment, the ligand is some type of antibody or part thereof which specifically binds to a luminally expressed, tissue-specific molecule. Usually, the ligand recognizes an epitope which does not participate in the binding of a natural ligand. The ligand of the luminally-expressed tissue-specific endothelial protein can be identified by any technique known to one of skill in the art, for example, using a two-hybrid technique, a combinatorial library, or producing an antibody molecule. The ligand may be a protein, RNA, DNA, small molecule or any other type of molecule which specifically binds to target proteins.

The target protein may be an integral membrane protein (such as a receptor) or may be a ligand itself. Should the tissue-specific molecule be a ligand which binds to a luminally expressed protein, the ligand, or a fragment thereof which exhibits the lumen and tissue-specificity, is used in the construction of the therapeutic complex of the invention. Alternatively, antibodies, antibody fragments, or antibody complexes specific to, or with similar binding characteristics to, the luminally exposed ligand molecule may be used in the construction of the therapeutic complex of the invention.

Should the tissue-specific luminally exposed protein (target protein) be a receptor, natural ligands can be identified by one of skill in the art in a number of different ways. For example, a two-hybrid technique can be used. Alternatively, high-throughput screening can be used to identify peptides which can act as ligands. Other methods of identifying ligand are known to one of skill in the art.

In one embodiment, the ligand of the therapeutic complex uses a different epitope than the natural ligand of the receptor target protein, so that there is no competition for binding sites.

In another embodiment, the ligand is an antibody molecule and preferably the antibody molecule has a higher specificity or binds to the tissue-specific luminally exposed receptor target protein in such a way that it will not be necessary to compete with the natural ligand.

Antibodies and fragments can be made by standard methods (See, for example, E. Harlow et al., Antibodies, A Laboratory Manual, Cold Spring Harbor Laboratory, Cold Spring Harbor, N.Y., 1988). However, the isolation, identification, and molecular construction of antibodies has been developed to such an extent that the choices are almost inexhaustible. Therefore, examples of antibody parts, and complexes will be provided with the understanding that this can only represent a sampling of what is available.

In one embodiment, the antibody is a single chain Fv region. Antibody molecules have two generally recognized regions, in each of the heavy and light chains. These regions are the so-called “variable” region which is responsible for binding to the specific antigen in question, and the so-called “constant” region which is responsible for biological effector responses such as complement binding, binding to neutrophils and macrophages, etc. The constant regions are not necessary for antigen binding. The constant regions have been separated from the antibody molecule, and variable binding regions have been obtained. Therefore, the constant regions are clearly not necessary for the binding action of the antibody molecule when it is acting as the ligand portion of the therapeutic complex.

The variable regions of an antibody are composed of a light chain and a heavy chain. Light and heavy chain variable regions have been cloned and expressed in foreign hosts, while maintaining their binding ability. Therefore, it is possible to generate a single chain structure from the multiple chain aggregate (the antibody), such that the single chain structure will retain the three-dimensional architecture of the multiple chain aggregate.

Fv fragments which are single polypeptide chain binding proteins having the characteristic binding ability of multi-chain variable regions of antibody molecules, can be used for the ligand of the present invention. These ligands are produced, for example, following the methods of Ladner et al., U.S. Pat. No. 5,260,203, issued Nov. 9, 1993, using a computer based system and method to determine chemical structures. These chemical structures are used for converting two naturally aggregated but chemically separated light and heavy polypeptide chains from an antibody variable region into a single polypeptide chain which will fold into a three dimensional structure very similar to the original structure of the two polypeptide chains. The two regions may be linked using an amino acid sequence as a bridge.

The single polypeptide chain obtained from this method can then be used to prepare a genetic sequence coding therefor. The genetic sequence can then be replicated in appropriate hosts, further linked to control regions, and transformed into expression hosts, wherein it can be expressed. The resulting single polypeptide chain binding protein, upon refolding, has the binding characteristics of the aggregate of the original two (heavy and light) polypeptide chains of the variable region of the antibody.

In a further embodiment, the antibodies are multivalent forms of single-chain antigen-binding proteins. Multivalent forms of single-chain antigen-binding proteins have significant utility beyond that of the monovalent single-chain antigen-binding proteins. A multivalent antigen-binding protein has more than one antigen-binding site which results in an enhanced binding affinity. The multivalent antibodies can be produced using the method disclosed in Whitlow et al., U.S. Pat. No. 5,869,620, issued Feb. 9, 1999. The method involves producing a multivalent antigen-binding protein by linking at least two single-chain molecules, each single chain molecule having two binding portions of the variable region of an antibody heavy or light chain linked into a single chain protein. In this way the antibodies can have binding sites for different parts of an antigen or have binding sites for multiple antigens.

In one embodiment, the antibody is an oligomer. The oligomer is produced as in PCT/EP97/05897, filed Oct. 24, 1997, by first isolating a specific ligand from a phage-displayed library. Oligomers overcome the problem of the isolation of mostly low affinity ligands from these libraries, by oligomerizing the low-affinity ligands to produce high affinity oligomers. The oligomers are constructed by producing a fusion protein with the ligand fused to a semi-rigid hinge and a coiled coil domain from Cartilage Oligomeric Matrix Protein (COMP). When the fusion protein is expressed in a host cell, it self assembles into oligomers.

Preferably, the oligomers are peptabodies (Terskikh et al., Biochemistry 94:1663-1668 (1997)). Peptabodies can be exemplified as IgM antibodies which are pentameric with each binding site having low-affinity binding, but able to bind in a high affinity manner as a complex. Peptabodies are made using phage-displayed random peptide libraries. A short peptide ligand from the library is fused via a semi-rigid hinge at the N-terminus of the COMP (cartilage oligomeric matrix protein) pentamerization domain. The fusion protein is expressed in bacteria where it assembles into a pentameric antibody which shows high affinity for its target. Depending on the affinity of the ligand, an antibody with very high affinity can be produced.

Preferably the antibody, antibody part or antibody complex of the present invention is derived from humans or is “humanized” (i.e. non-immunogenic in a human) by recombinant or other technology. Such antibodies are the equivalents of the monoclonal and polyclonal antibodies disclosed herein, but are less immunogenic, and are better tolerated by the patient.

Humanized antibodies may be produced, for example, by replacing an immunogenic portion of an antibody with a corresponding, but non-immunogenic portion (i.e. chimeric antibodies) (See, for example, Robinson, et al., PCT Application No. PCTIUS86/02269; Akira, et al., European Patent Application No. 184,187; Taniguchi, European Patent Application No. 171,496; Morrison, et al., European Patent Application No. 173,494; Neuberger, et al., International Patent Publication No. WO86/01533; Cabilly, et al., European Patent Application No. 125,023; Better, et al., Science 240:1041-1043 (1988); Liu, et al., Proc. Natl. Acad. Sci. USA 84:3439-3433 (1987); Liu, et al., J. Immunol. 139:3521-3526 (1987); Sun, et al., Proc. Natl. Acad. Sci. USA 84:214-218 (1987); Nishimura, et al., Canc. Res. 47:999-1005 (1987); Wood, et al., Nature 314:446-449 (1985)); Shaw et al., J. Natl. Cancer Inst. 80:1553-1559 (1988); all of which references are incorporated herein by reference). General reviews of “humanized” chimeric antibodies are provided by Morrison, (Science, 229:1202-1207 (1985)) and by Oi, et al., BioTechniques 4:214 (1986); which references are incorporated herein by reference).

Suitable “humanized” antibodies can be alternatively produced by CDR or CEA substitution (Jones, et al., Nature 321:552-525 (1986); Verhoeyan et al., Science 239:1534 (1988); Bsidler, et al., J. Immunol. 141:4053-4060 (1988); all of which references are incorporated herein by reference.

Small molecules are any non-biopolymeric DNA, RNA, organic, or inorganic molecules such as macrocycles, alkene isomers, and many of what is typically thought of as drugs in the pharmaceutical industry. These molecules are often identified through combinatorial processes. In particular, a ligand can be identified using a process called “docking”, an approach to rational drug design which seeks to predict the structure and binding free energy of a ligand-receptor complex given only the structures of the free ligand and receptor. Typically, these small molecules are used to bind to a specific protein and elicit an effect. However, it is envisioned in this context that they would simply be used to bind a specific protein and thus localize the attached drug to the required organs.

### Linkers

The “linker” as used herein is any bond, small molecule, or other vehicle which allows the ligand and the therapeutic moiety to be targeted to the same area, tissue, or cell. Preferably, the linker is cleavable.

In one embodiment the linker is a chemical bond between one or more ligands and one or more therapeutic moieties. Thus, the bond may be covalent or ionic. An example of a therapeutic complex where the linker is a chemical bond would be a fusion protein. In one embodiment, the chemical bond is acid sensitive and the pH sensitive bond is cleaved upon going from the blood stream (pH 7.5) to the transcytotic vesicle or the interior of the cell (pH about 6.0). Alternatively, the bond may not be acid sensitive, but may be cleavable by a specific enzyme or chemical which is subsequently added or naturally found in the microenvironment of the targeted site. Alternatively, the bond may be a bond that is cleaved under reducing conditions, for example a disulfide bond. Alternatively, the bond may not be cleavable.

Any kind of acid cleavable or acid sensitive linker may be used. Examples of acid cleavable bonds include, but are not limited to: a class of organic acids known as cis-polycarboxylic alkenes. This class of molecule contains at least three carboxylic acid groups (COOH) attached to a carbon chain that contains at least one double bond. These molecules as well as how they are made and used is disclosed in Shen, et al. U.S. Pat. No. 4,631,190 (herein incorporated by reference). Alternatively, molecules such as amino-sulfhydryl cross-linking reagents which are cleavable under mildly acidic conditions may be used. These molecules are disclosed in Blattler et al., U.S. Pat. No. 4,569,789 (herein incorporated by reference).

Alternatively, the acid cleavable linker may be a time-release bond, such as a biodegradable, hydrolyzable bond. Typical biodegradable carrier bonds include esters, amides or urethane bonds, so that typical carriers are polyesters, polyamides, polyurethanes and other condensation polymers having a molecular weight between about 5,000 and 1,000,000. Examples of these carriers/bonds are shown in Peterson, et al., U.S. Pat. No. 4,356,166 (herein incorporated by reference). Other acid cleavable linkers may be found in U.S. Pat. Nos. 4,569,789 and 4,631,190 (herein incorporated by reference) or Blattner et al. in Biochemistry 24: 1517-1524 (1984). The linkers are cleaved by natural acidic conditions, or alternatively, acid conditions can be induced at a target site as explained in Abrams et al., U.S. Pat. No. 4,171,563 (herein incorporated by reference).

Examples of linking reagents which contain cleavable disulfide bonds (reducable bonds) include, but are not limited to “DPDPB”, 1,4- di-[3′-(2′-pyridyldithio)propionamido]butane; “SADP”, (N-succinimidyl(4-azidophenyl)1,3′-dithiopropionate); “Sulfo-SADP” (Sulfosuccinimidyl (4-azidophenyldithio)propionate; “DSP”-Dithio bis (succinimidylproprionate); “DTSSP”-3,3′-Dithio bis (sulfosuccinimidylpropionate); “DTBP”-dimethyl 3,3′-dithiobispropionimidate-2 HCl, all available from Pierce Chemicals (Rockford, Ill.).

Examples of linking reagents cleavable by oxidation are “DST”-disuccinimidyl tartarate; and “Sulfo-DST”-disuccinimidyl tartarate. Again, these linkers are available from Pierce Chemicals.

Examples of non-cleavable linkers are “Sulfo-LC-SMPT”-(sulfosuccinimidyl 6-[alpha-methyl-alpha-(2-pyridylthio)toluamido}hexanoate;“SMPT”; “ABH”-Azidobenzoyl hydrazide; “NHS-ASA”-N-Hydroxysuccinimidyl-4-azidosalicyclic acid; “SASD”-Sulfosuccinimidyl 2-(p-azidosalicylamido)ethyl-1,3-dithiopropionate; “APDP”-N-{4-(p-azidosalicylamido) buthy}-3′(2′-pyidyldithio) propionamide; “BASED”-Bis-[beta-(4-azidosalicylamido)ethyl] disulfide; “HSAB”-N-hydroxysuccinimidyl-4 azidobenzoate; “APG”-p-Azidophenyl glyoxal monohydrate; “SANPAH”-N-Succiminidyl -6(4′-azido-2′-mitrophenyl -amimo)hexanoate; “Sulfo-SANPAH”-Sulfosuccinimidyl 6-(4 ′-azido-2′-nitrophenylamino)hexanoate; “ANB-NOS”-N-5-Azido-2-nitrobenzyoyloxysuccinimide; “SAND”-Sulfosuccinimidyl-2-(m-azido-o-mitrobenzamido)-ethyl-1,3′-dithiopropionate; “PNP-DTP”-p-nitrophenyl-2-diazo-3,3,3-trifluoropropionate; “SMCC”-Succinimidyl 4-(N-maleimidomethyl)cyclohexane-1-carboxylate; “Sulfo-SMCC”-Sulfosuccinimidyl 4-(N-maleimidomethyl)cyclohexane-1-carboxylate; “MBS”-m-Maleimidobenzoyl-N-hydroxysuccinimide ester; “sulfo-MBS”-m-Maleimidobenzoyl-N-hydroxysulfosuccinimide ester; “SIAB”-N-Succinimidyl(4-iodoacetyl)aminobenzoate; “Sulfo-SIAB”-N-Sulfosuccinimidyl(4-iodoacetyl)aminobenzoate; “SMPB”-Succinimidyl 4-(p-malenimidophenyl)butyrate; “Sulfo-SMPB”-Sulfosuccinimidyl 4-(p-malenimidophenyl)butyrate; “DSS”-Disuccinimidyl suberate; “BSSS”-bis(sulfosuccinimidyl) suberate; “BMH”-Bis maleimidohexane; “DFDNB”-1,5-difluoro-2,4-dinitrobenzene; “DMA”-dimethyl adipimidate 2 HCl; “DMP”-Dimethyl pimelimidate -2HCl; “DMS”-dimethyl suberimidate-2-HCl; “SPDP”-N-succinimidyl-3-(2-pyridylthio)propionate; “Sulfo -HSAB”-Sulfosuccinimidyl 4-(p-azidophenyl)butyrate; “Sulfo-SAPB”-Sulfosuccinimidyl 4-(p-azidophenylbutyrate); “ASIB”-1-9p-azidosalicylamido)-4-(iodoacetamido)butane; “ASBA”-4-(p-Azidosalicylamido)butylamine. All of these linkers are available from Pierce Chemicals.

In another embodiment the linker is a small molecule such as a peptide linker. In one embodiment the peptide linker is not cleavable. In a further embodiment the peptide linker is cleavable by base, under reducing conditions, or by a specific enzyme. In one embodiment, the enzyme is indigenous. Alternatively, the small peptide may be cleavable by an non-indigenous enzyme which is administered after or in addition to the therapeutic complex. Alternatively, the small peptide may be cleaved under reducing conditions, for example, when the peptide contains a disulfide bond. Alternatively, the small peptide may be pH sensitive. Examples of peptide linkers include: poly(L-Gly), (Poly L-Glycine linkers); poly(L-Glu), (Poly L-Glutamine linkers); poly(L-Lys), (Poly L-Lysine linkers). In one embodiment, the peptide linker has the formula (amino acid)ₙ, where n is an integer between 2 and 100, preferably wherein the peptide comprises a polymer of one or more amino acids.

In a further embodiment, the peptide linker is cleavable by proteinase such as one having the sequence Gly-(D)Phe-Pro-Arg-Gly-Phe-Pro-Ala-Gly-Gly (SEQ ID NO: 1) (Suzuki, et al. 1998, J. Biomed. Mater. Res. Oct;42(1):112-6). This embodiment has been shown to be advantageous for the treatment of bacterial infections, particularly Pseudomonas aeruginosa. Gentamicin or an alternate antibiotic is cleaved only when the wounds are infected by Pseudomonas aeruginosa because there is significantly higher activity of thrombin-like proteinase enzymes then in non-infected tissue.

In a further embodiment the linker is a cleavable linker comprising, poly(ethylene glycol) (PEG) and a dipeptide, L-alanyl-L-valine (Ala-Val), cleavable by the enzyme thermolysin. This linker is advantageous because thermolysin-like enzyme has been reported to be expressed at the site of many tumors. Alternatively, a 12 residue spacer Thr-Arg-His-Arg-Gln-Pro-Arg-Gly-Trp-Glu-Gln-Leu (SEQ ID NO:2) may be used which contains the recognition site for the protease furin (Goyal, et al. Biochem. J. Jan. 15, 2000; 345 Pt 2:247-254).

The chemical and peptide linkers can be bonded between the ligand and the therapeutic moiety by techniques known in the art for conjugate synthesis, i.e. using genetic engineering, or chemically. The conjugate synthesis can be accomplished chemically via the appropriate antibody by classical coupling reactions of proteins to other moieties at appropriate functional groups. Examples of the functional groups present in proteins and utilized normally for chemical coupling reactions are outlined as follows. The carbohydrate structures may be oxidized to aldehyde groups that in turn are reacted with a compound containing the group H₂NNH-R (wherein R is the compound) to the formation of a C═NH—NH—R group. The thiol group (cysteines in proteins) may be reacted with a compound containing a thiol-reactive group to the formation of a thioether group or disulfide group. The free amino group (at the amino terminus of a protein or on a lysine) in amino acid residues may be reacted with a compound containing an electrophilic group, such as an activated carboxy group, to the formation of an amide group. Free carboxy groups in amino acid residues may be tranformed to a reactive carboxy group and then reacted with a compound containing an amino group to the formation of an amide group.

The linker may alternatively be a liposome. Many methods for the preparation of liposomes are well known in the art. For example, the reverse phase evaporation method, freeze-thaw methods, extrusion methods, and dehydration-rehydration methods. (see Storm, et al. PSTT 1:19-31 (1998), the disclosure of which is incorporated herein by reference in its entirety).

The liposomes may be produced in a solution containing the therapeutic moiety so that the substance is encapsulated during polymerization. Alternatively, the liposomes can be polymerized first, and the biologically active substance can be added later by resuspending the polymerized liposomes in a solution of a biologically active substance and treating with sonication to affect encapsulation of the therapeutic moiety. The liposomes can be polymerized in the presence of the ligand such that the ligand becomes a part of the phospholipid bilayer. In one embodiment, the liposome contains the therapeutic moiety on the inside and the ligand on the outside.

The liposomes contemplated in the present invention can comprise a variety of structures. For example, the liposomes can be multilamellar large vesicles (MLV), oligolamellar vesicles (OLV), unilamellar vesicles (UV), small unilamellar vesicles (SUV), medium sized unilamellar vesicles (MUV), large unilamellar vesicles (LUV), giant unilamellar vesicles (GUV), or multivesicular vesicles (MVV). Each of these liposome structures are well known in the art (see Storm, et al. PSTT 1:19-31 (1998), the disclosure of which is incorporated herein by reference in its entirety).

In one embodiment, the liposome is a “micromachine” that evulses pharmaceuticals for example by the application of specific frequency radio waves. In another embodiment, the liposomes can be degraded such that they will release the therapeutic moiety in the targeted cell, for example, the liposomes may be acid or alkaline sensitive, or degraded in the presence of a low or high pH, such that the therapeutic moiety is released within the cell. Alternatively, the liposomes may be uncharged so that they will be taken up by the targeted cell. The liposomes may also be pH sensitive or sensitive to reducing conditions.

One type of liposome which may be advantageously used in the present invention is that identified in Langer et al., U.S. Pat. No. 6,004,534, issued Dec. 21, 1999 (herein incorporated by reference). In this application a method of producing modified liposomes which are prepared by polymerization of double and triple bond-containing monomeric phospholipids is disclosed. These liposomes have surprisingly enhanced stability against the harsh environment of the gastointestinal tract. Thus, they have utility for oral and/or mucosal delivery of the therapeutic moiety. It has also been shown that the liposomes may be absorbed into the systemic circulation and lymphatic circulation. The liposomes are generally prepared by polymerization (i.e., radical initiation or radiation) of double and triple bond-containing monomeric phospholipids.

In other embodiments of the present invention, the linker can also be a liposome having a long blood circulation time. Such liposomes are well known in the art, (see U.S. Pat. No., 5,013,556; 5,225,212; 5,213,804; 5,356,633; and 5,843,473, the disclosures of which are incorporated herein by reference in their entireties). Liposomes having long blood circulation time are characterized by having a portion of their phosphoslipids derivatized with polyethylene glycol (PEG) or other similar polymer. In some embodiments, the end of the PEG molecule distal to the phospholipid may be activated so a to be chemically reactive. Such a reactive PEG molecule can be used to link a ligand to the liposome. One example of a reactive PEG molecule is the maleimide derivative of PEG described in U.S. Pat. No. 5,527,528, the disclosure of which is incorporated herein by reference in its entirety).

Alternatively, the linker may be a microcapsule, a nanoparticle, a magnetic particle, and the like (Kumar, J. Pharm. Sci., May-August 3(2)234-258, 2000; and Gill et al., Trends Biotechnol. November; 18(11):469-79, 2000), with the lipophillic therapeutic moiety on or in the container, and the container functioning as the linker in the therapeutic complex.

Alternatively, the linker may be a photocleavable linker. For example, a 1-2-(nitrophenyl)-ethyl moiety can be cleaved using 300 to 360 nm light (see Pierce catalog no. 21332ZZ). It can be envisioned that the photocleavable linker would allow activation and action of the drug in an even more specific area, for example a particular part of the organ. The light could be localized using a catheter into the vessel. Alternatively, light may be used to localize treatment to a specific part of the digestive tract and the light may be manipulated through a natural orifice to the area. Alternatively, the light can be surgically manipulated to the area.

Alternatively, the linker may not be cleavable, but the therapeutic moiety or ligand is. An example of this is when the therapeutic moiety is a prodrug and the enzyme which cleaves the prodrug is administered with the therapeutic complex. Alternatively, the enzyme is part of the therapeutic complex or indigenous and the prodrug is administered separately. Preferably, the enzyme or prodrug which is administered separately is administered within about 48 hours of the first administration. Alternatively, the prodrug or enzyme which is administered separately may be administered between about 1 min and 24 hours, alternatively between about 2 min and 8 hours. The prodrug or enzyme which is administered separately, may be readministered at a later date and may continue to be administered until the effect of the drug is not longer needed or until the enzymatic cleavage of all of the drug is effected.

### Therapeutic Moieties

The “therapeutic moiety” could be any chemical, molecule, or complex which effects a desired result. Examples include but are not limited to: conventional pharmaceutical agents such as antibiotics, anti-neoplastic agents, immunosuppressive agents, hormones, and the like, one or more genes, antisense oligonucleotides, contrast agents, proteins, toxins, radioactive molecules or atoms, surfactant proteins, or clotting proteins. The therapeutic moiety may be lipophilic, a quality which will help it enter the targeted cell.

The contrast agents may be any type of contrast agent known to one of skill in the art. The most common contrast agents basically fall into one of four groups; X-ray reagents, radiography reagents, magnetic resonance imaging agents, and ultrasound agents. The X-ray reagents include ionic, iodine-containing reagents as well as non-ionic agents such as Omnipaque (Nycomed) and Ultravist (Schering). Radiographic agents include radioisotopes as disclosed below. Magnetic Resonance Imaging reagents include magnetic agents such a Gadolinium and iron-oxide chelates. Ultrasound agents include microbubbles of gas and a number of bubble-releasing formulations.

The radionuclides may be diagnostic or therapeutic. Examples of radionuclides that are generally medically useful include: Y, Ln, Cu, Lu, Tc, Re, Co, Fe and the like such as ⁹⁰Y, ¹¹¹Ln, ⁶⁷Cu, ⁷⁷Lu, ⁹⁹Tc and the like, preferably trivalent cations, such as ⁹⁰Y and ¹¹¹Ln.

Radionuclides that are suitable for imaging organs and tissues in vivo via diagnostic gamma scintillation photometry include the following: γ-emitting radionuclides: ¹¹¹Ln, ^{113m}Ln, ⁶⁷Ga, ⁶⁸Ga, ^{99m}Tc, ⁵¹Cr, ¹⁹⁷Hg, ²⁰³Hg, ¹⁶⁹Yb, ⁸⁵Sr, and ⁸⁷Sr. The preparation of chelated radionuclides that are suitable for binding by Fab′ fragments is taught in U.S. Pat. No. 4,658,839 (Nicoletti et al.) which is incorporated herein by reference.

Paramagnetic metal ions, suitable for use as imaging agents in MRI include the lanthanide elements of atomic number 57-70, or the transition metals of atomic numbers 21-29, 42 or 44. U.S. Pat. No. 4,647,447 (Gries et al.) teaches MRI imaging via chelated paramagnetic metal ions and is incorporated herein by reference.

Examples of therapeutic radionuclides are the β-emitters. Suitable β-emitters include ⁶⁷Cu, ¹⁸⁶Rh, ¹⁸⁸Rh , ¹⁵³Sm, ⁹⁰Y, and ¹¹¹Ln.

Antisense oligonucleotides have a potential use in the treatment of any disease caused by overexpression of a normal gene, or expression of an aberrant gene. Antisense oligonucleotides can be used to reduce or stop expression of that gene. Examples of oncogenes which can be treated with antisense technology and references which teach specific antisense molecules which can be used include: c-Jun and cFos (U.S. Pat. No. 5,985,558, herein incorporated by reference); HER-2 (U.S. Pat. No. 5,968,748, herein incorporated by reference) E2F-1 (Popoff, et al. U.S. Pat. No. 6,187,587; herein incorporated by reference), SMAD 1-7 (U.S. Pat. Nos. 6,159,697; 6,013,788; 6,013,787; 6,013,522; and 6,037,142, herein incorporated by reference), and Fas (Dean et al. U.S. Pat. No. 6,204,055, herein incorporated by reference).

Proteins which may be used as therapeutic agents include apoptosis inducing agents such as pRB and p53 which induce apoptosis when present in a cell (Xu et al. U.S. Pat. No. 5,912,236, herein incorporated by reference), and proteins which are deleted or underexpressed in disease such as erythropoietin (Sytkowski, et al. U.S. Pat. No. 6,048,971, herein incorporated by reference)

It can be envisioned that the therapeutic moiety can be any chemotherapeutic agent for neoplastic diseases such as alkylating agents (nitrogen mustards, ethylenimines, alkyl sulfonates, nitrosoureas, and triazenes), antimetabolites (folic acid analogs such as methotrexate, pyrimidine analogs, and purine analogs), natural products and their derivatives (antibiotics, alkaloids, enzymes), hormones and antagonists (adrenocorticosteroids, progestins, estrogens), and the like. Alternatively, the therapeutic moiety can be an antisense oligonucleotide which acts as an anti-neoplastic agent, or a protein which activates apoptosis in a neoplastic cell.

The therapeutic moiety can be any type of neuroeffector, for example, neurotransmittors or neurotransmitter antagonists may be targeted to an area where they are needed without the wide variety of side effects commonly experienced with their use.

The therapeutic moiety can be an anesthetic such as an opioid, which can be targeted specifically to the area of pain. Side effects, such as nausea, are commonly experienced by patients using opioid pain relievers. The method of the present invention would allow the very specific localization of the drug to the area where it is needed, such as a surgical wound or joints in the case of arthritis, which may reduce the side effects.

The therapeutic moiety can be an anti-inflammatory agent such as histamine, H₁-receptor antagonists, and bradykinin. Alternatively, the anti-inflammatory agent can be a non-steroidal anti-inflammatory such as salicylic acid derivatives, indole and indene acetic acids, and alkanones. Alternatively, the anti-inflammatory agent can be one for the treatment of asthma such as corticosteroids, cromollyn sodium, and nedocromil. The anti-inflammatory agent can be administered with or without the bronchodilators such as B₂-selective andrenergic drugs and theophylline.

The therapeutic moiety can be a diuretic, a vasopressin agonist or antagonist, angiotensin, or renin which specifically effect a patient's blood pressure.

The therapeutic moiety can be any pharmaceutical used for the treatment of heart disease. Such pharmaceuticals include, but are not limited to, organic nitrites (amyl nitrites, nitroglycerin, isosorbide dinitrate), calcium channel blockers, antiplatelet and antithrombotic agents, vasodilators, vasoinhibitors, anti-digitalis antibodies, and nodal blockers.

The therapeutic moiety can be any pharmaceutical used for the treatment of protozoan infections such as tetracycline, clindamycin, quinines, chloroquine, mefloquine, trimethoprimsulfamethoxazole, metronidazole, and oramin. The ability to target pharmaceuticals or other therapeutics to the area of the protozoal infection is of particular value due to the very common and severe side effects experienced with these antibiotic pharmaceuticals.

The therapeutic moiety can be any anti-bacterial such as sulfonamides, quinolones, penicillins, cephalosporins, aminoglycosides, tetracyclines, chloramphenicol, erythromycin, isoniazids and rifampin.

The therapeutic moiety can be any pharmaceutical agent used for the treatment of fungal infections such as amphotericins, flucytosine, miconazole, and fluconazole.

The therapeutic moiety can be any pharmaceutical agent used for the treatment of viral infections such as acyclovir, vidarabine, interferons, ribavirin, zidovudine, zalcitabine, reverse transcriptase inhibitors, and protease inhibitors. It can also be envisioned that virally infected cells can be targeted and killed using other therapeutic moieties, such as toxins, radioactive atoms, and apoptosis-inducing agents.

The therapeutic moiety can be chosen from a variety of anticoagulant, anti-thrombolyic, and anti-platelet pharmaceuticals.

It can be envisioned that diseases resulting from an over- or under-production of hormones can be treated using such therapeutic moieties as hormones (growth hormone, androgens, estrogens, gonadotropin-releasing hormone, thyroid hormones, adrenocortical steroids, insulin, and glucagon). Alternatively, if the hormone is over-produced, antagonists or antibodies to the hormones may be used as the therapeutic moiety.

Various other possible therapeutic moieties include vitamins, enzymes, and other under-produced cellular components and toxins such as diptheria toxin or botulism toxin.

Alternatively, the therapeutic moiety may be one that is typically used in in vitro diagnostics. Thus, the ligand and linker are labeled by conventional methods to form all or part of a signal generating system. The ligand and linker can be covalently bound to radioisotopes such as tritium, carbon 14, phosphorous 32, iodine 125 and iodine 131 by methods well known in the art. For example, ¹²⁵I can be introduced by procedures such as the chloramine-T procedure, enzymatically by the lactoperoxidase procedure or by the prelabeled Bolton-Hunter technique. These techniques plus others are discussed in H. Van Vunakis and J. J. Langone, Editors, Methods in Enzymology, Vol. 70, Part A, 1980. See also U.S. Pat. No. 3,646,346, issued Feb. 29, 1972, and Edwards et al., U.S. Pat. No. 4,062,733, issued Dec. 13, 1977, respectively, both of which are herein incorporated by reference, for further examples of radioactive labels.

Therapeutic moieties also include chromogenic labels, which are those compounds that absorb light in the visible ultraviolet wavelengths. Such compounds are usually dyestuffs and include quinoline dyes, triarylmethane dyes, phthaleins, insect dyes, azo dyes, anthraquimoid dyes, cyanine dyes, and phenazoxonium dyes.

Fluorogenic compounds can also be therapeutic moieties and include those which emit light in the ultraviolet or visible wavelength subsequent to irradiation by light. The fluorogens can be employed by themselves or with quencher molecules. The primary fluorogens are those of the rhodamine, fluorescein and umbelliferone families. The method of conjugation and use for these and other fluorogens can be found in the art. See, for example, J. J. Langone, H. Van Vunakis et al., Methods in Enzymology, Vol. 74, Part C, 1981, especially at page 3 through 105. For a representative listing of other suitable fluorogens, see Tom et al., U.S. Pat. No. 4,366,241, issued Dec. 28, 1982, especially at column 28 and 29. For further examples, see also U.S. Pat. No. 3,996,345, herein incorporated by reference.

These non-enzymatic signal systems are adequate therapeutic moieties for the present invention. However, those skilled in the art will recognize that an enzyme-catalyzed signal system is in general more sensitive than a non-enzymatic system. Thus, for the instant invention, catalytic labels are the more sensitive non-radioactive labels.

Catalytic labels include those known in the art and include single and dual (“channeled”) enzymes such as alkaline phosphatase, horseradish peroxidase, luciferase, β-galactosidase, glucose oxidase (lysozyme, malate dehydrogenase, glucose-6-phosphate dehydrogenase) and the like. Examples of dual (“channeled”) catalytic systems include alkaline phosphatase and glucose oxidase using glucose-6-phosphate as the initial substrate. A second example of such a dual catalytic system is illustrated by the oxidation of glucose to hydrogen peroxide by glucose oxidase, which hydrogen peroxide would react with a leuco dye to produce a signal generator. (A further discussion of catalytic systems can be found in Tom et al., U.S. Pat. No. 4,366,241, issued Dec. 28, 1982, herein incorporated by reference (see especially columns 27 through 40). Also, see Weng et al., U.S. Pat. No. 4,740,468, issued Apr. 26, 1988, herein incorporated by reference, especially at columns 2 and columns 6, 7 and 8.

The procedures for incorporating enzymes into the instant therapeutic complexes are well known in the art. Reagents used for this procedure include glutaraldehyde, p-toluene diisocyanate, various carbodiimide reagents, p-benzoquinone m-periodate, N,N¹-o-phenylenedimaleimide and. the like (see, for example, J. H. Kennedy et al., Clin. Chim Acta 70, 1 (1976)). As another aspect of the invention, any of the above devices and formats may be provided in a kit in packaged combination with predetermined amounts of reagents for use in assaying for a tissue-specific endothelial protein.

Chemiluminescent labels are also applicable as therapeutic moieties. See, for example, the labels listed in C. L. Maier, U.S. Pat. No. 4,104,029, issued Aug. 1, 1978, herein incorporated by reference.

The substrates for the catalytic systems discussed above include simple chromogens and fluorogens such as para-nitrophenyl phosphate (PNPP), β-D-glucose (plus possibly a suitable redox dye), homovanillic acid, o-dianisidine, bromocresol purple powder, 4-alkyl-umbelliferone, luminol, para-dimethylaminolophine, paramethoxylophine, AMPPD, and the like.

Depending on the nature of the label and catalytic signal producing system, one would observe the signal by irradiating with light and observing the level of fluorescence; providing for a catalyst system to produce a dye, fluorescence, or chemiluminescence, where the dye could be observed visually or in a spectrophotometer and the fluorescence could be observed visually or in a fluorimeter; or in the case of chemiluminescence or a radioactive label, by employing a radiation counter. Where the appropriate equipment is not available, it will normally be desirable to have a chromophore produced which results in a visible color. Where sophisticated equipment is involved, any of the techniques are applicable.

Alternatively, the therapeutic moiety can be a prodrug or a promolecule which is converted into the corresponding pharmaceutical agent by a change in the chemical environment or by the action of a discrete molecular agent, such as an enzyme. Preferably, the therapeutic moiety is administered with the specific molecule needed for conversion of the promolecule. Alternatively, the promolecule can be cleaved by a natural molecule found in the microenvironment of the target tissue. Alternatively, the prodrug is pH sensitive and converted upon change in environment from the blood to the cell or vesicle (Greco et al., J. Cell. Physiol. 187:22-36, 2001).

### Uses of the Therapeutic Complexes

The therapeutic complex may be used to treat or diagnose any disease for which a tissue- or organ-specific treatment would be efficacious. Examples of such tissues and diseases follow:

In one embodiment, the therapeutic complex may be used to treat or alleviate the symptoms of diseases which affect the brain. Examples of such diseases include but are not limited to: bacterial infections, viral infections, fungal and parasitic infections, epilepsy, schizophrenia, bipolar disorder, neurosis, depression, brain cancer, Parkinson's disease, Alzheimer's disease and other forms of dementia, prion-related diseases, stroke, migraine, ataxia, multiple sclerosis, meningitis, brain abscess, and Wernicke's disease or other metabolic disorders.

In a further embodiment, the therapeutic complex may be used to treat diseases which affect the lungs. Examples- of such diseases include but are not limited to: bacterial infections (i.e. S. pneumoniae, M. tuberculosis), viral infections (i.e. Hantavirus), fungal and parasitic infections (i.e. Pneumocystis carinii), asthma, lung cancer, emphysema, lung transplant rejection, cystic fibrosis, pulmonary hypertension, pulmonary thromboembolism, and pulmonary edema.

In a further embodiment, the therapeutic complex may be used to treat or alleviate the symptoms of diseases which affect the pancreas. Examples of such diseases include but are not limited to: parasitic infections, pancreatic cancer, chronic pancreatitis, and pancreatic insufficiency, endocrine tumors, and diabetes.

In one embodiment, the therapeutic complex may be used to treat or alleviate the symptoms of diseases which affect the kidney. Examples of such diseases include but are not limited to: bacterial infections, viral infections, fungal and parasitic infections, polycystic kidney disease, kidney transplant rejection, edema, hypertension, hypervolemia, bladder and renal cell cancer and uremic syndrome.

In one embodiment, the therapeutic complex may be used to treat or alleviate the symptoms of diseases which affect the muscles. Examples of such diseases include but are not limited to: muscular dystrophy, polymyositis, arthritic diseases, rhabdomyosarcoma, polymyositis, disorders of glycogen storage, and soft tissue sarcomas.

In one embodiment, the therapeutic complex may be used to treat or alleviate the symptoms of diseases which affect the gut or intestine. Examples of such diseases include but are not limited to: dysentery, gastroenteritis, irritable bowel disease, diverticulosis/diverticulitis, peptic ulcer, cryptosporidiosis, giardiasis, inflammatory bowel disease, colorectal cancer, and tumors of the small intestine.

In one embodiment, the therapeutic complex may be used to treat or alleviate the symptoms of diseases which affect the prostate. Examples of such diseases include but are not limited to: hyperplasia of the prostate, prostate cancer, and infections of the prostate.

In a further embodiment, the therapeutic complex may be used as a diagnostic of disease or tissue type or to quantify or identify the tissue-specific luminally expressed protein.

The cells bearing target proteins interact with the therapeutic complex in two general ways, by transcytosis or passive diffusion. These interactions allow the therapeutic complex to interact directly with the vascular endothelial cell bearing the target protein, become enmeshed in the endothelial matrix containing said endothelial cell, or cross through the endothelial matrix into the encapsulated tissue or organ.

Transcytosis occurs when, after attachment of the complex with the target protein on the endothelial cell, the therapeutic complex is transcytosed across the vasculature into the endothelial matrix tissue or endothelial cell of choice. Preferably, the binding of the ligand to the target protein will stimulate the transport of the therapeutic complex across the endothelium within a transcytotic vesicle. During transcytosis, the conditions within the microenvironment of the vesicle are more highly acidic and can be used to selectively cleave the therapeutic moiety. For this to happen, preferably, the linker should be pH sensitive, so as to be cleaved due to the change in pH upon going from the blood stream (pH 7.5) to transcytotic vesicles or the interior of the cell (pH 6.0) such as the acid sensitive linkers disclosed. Alternatively, a separate linker may not be necessary when the bond between the ligand and the therapeutic moiety is itself acid sensitive.

In passive diffusion, the ligand in the complex may attach to the exterior cell membrane, following which there is release of the therapeutic moiety which crosses into the endothelial cell or tissue by passive means, but there is no entry of the entire therapeutic complex into the cell. Preferably, the therapeutic agent is released in high concentrations in microproximity to the endothelium within the specific target tissue. These higher concentrations are expected to result in relatively greater concentrations of the drug reaching the target tissue versus systemic tissues.

The therapeutic complexes may be taken up by the cell and stay within the cell or cellular matrix or may cross into the organs and become diffuse within the organ.

The therapeutic complexes of the present invention advantageously bind to a target protein on a specific tissue, organ or cell and can be used for a number of desired outcomes. In one embodiment, the therapeutic complexes are used to keep toxic substances in a specific environment, allowing for a more specific targeting of a therapeutic moiety to that environment and preventing systemic effects of the therapeutic moiety. In addition, a lower concentration of the substance would be needed for the same effect.

In a further embodiment, the therapeutic complex is used to keep substances from getting into tissues. The therapeutic moiety might be used to block receptors, that if activated, would cause further harm to the surrounding tissue.

In a further embodiment the therapeutic complex is used to replace a substance, such as a surfactant protein, or a hormone which is in some way dysfunctional or absent from a specific tissue.

### Prodrugs

The concept of prodrugs are well known in the art and are used herein in a similar manner. The instant prodrugs possess different pharmaceutical characteristics before and after their conversion from prodrug to the corresponding pharmaceutical agent. The therapeutic complexes of the present invention may advantageously incorporate the use of a prodrug in two ways. The therapeutic complexes may have a prodrug attached as a therapeutic moiety which can be converted either by the subsequent injection of a non-indigenous enzyme, or by an enzyme found in the tissue of choice. Alternatively, the therapeutic moiety may be the enzyme which is needed to convert the prodrug. For example, the enzyme β-lactamase may be a part of the therapeutic complex and the prodrug (i.e., doxocillin) is subsequently added and, because the β-lactamase is only found in the targeted tissue, the doxocillin is only unmasked in that area. Unfortunately, neoplastic tissues usually share the enzyme repertoire of normal tissues, making the use of an indigenous enzyme less desirable. However, it can be envisioned that diseased tissues, particularly those diseased by pathogens, may be producing an enzyme specific to the pathogen which is infecting the tissue and this could be used to design an effective prodrug treatment which would be very specific to the infected tissue. For example, a prodrug which is converted by a viral enzyme (i.e., HBV) could be used with a liver-specific antiviral therapeutic complex to get very specific antiviral effect because the prodrug would only be converted in the microenvironment containing the virus.

Therefore, in one embodiment, a “ligand-enzyme” therapeutic complex is used in combination with the unattached prodrug. The prodrug is cleaved by an enzyme and enters the cell. Preferably, the prodrug is hydrophilic, blocking its access into endothelial cells, while the (cleaved) drug is lypophilic, enhancing its ability to enter cells. Alternatively, a “ligand-prodrug” is used as the therapeutic complex in combination with the administration of an unattached non-indigenous enzyme or an indigenous enzyme. The prodrug is cleaved by the enzyme, thus, separated from the therapeutic wherein its lipophilic qualities allow it to enter the cell.

Two of the advantages of the prodrug approach include bystander killing and amplification. One problem with the previous use of antibodies or immunoconjugates in the treatment of cancer was that they were inefficiently taken up by the cells and poorly localized. However, when using a prodrug treatment, because a single molecule of enzyme can convert more than one prodrug molecule the chance of uptake is increased or amplified considerably. In addition, as the active drug diffuses throughout the tumor, it provides a bystander effect, killing or otherwise effecting the therapeutic action on antigen-negative, abnormal cells. Although this bystander effect may also effect normal cells, they will only be those in the direct vicinity of the tumor or diseased organ.

A number of prodrugs have been widely used for cancer therapy and are presented below as examples of prodrugs which can be used in the present invention (Greco et al., J. Cell. Phys. 187:22-36, 2001; and Konstantinos et al., Anticancer Research 19:605-614, 1999). However, it is to be understood that these are some of many examples of this embodiment of the invention.

The most well-studied enzyme/prodrug combination is Herpes simplex virus thymidine kinase (HSV TK) with the nucleotide analog GCV. GCV and related agents are poor substrates for the mammalian nucleoside monophosphate kinase, but can be converted (1000 fold more) efficiently to the monophosphate by TK from HSV 1. Subsequent reactions catalyzed by cellular enzymes lead to a number of toxic metabolites, the most active ones being the triphosphates. GCV-triphosphate competes with deoxyguanosine triphosphate for incorporation into elongating DNA during cell division, causing inhibition of the DNA polymerase and single strand breaks.

The system consisting of cytosine deaminase and 5-fluorocytosine (CD and 5-FC respectively) is similarly based on the production of a toxic nucleotide analog. The enzyme CD, found in certain bacteria and fungi but not in mammalian cells, catalyses the hydrolytic deamination of cytosine to uracil. It can therefore convert the non-toxic prodrug 5-FC to 5-fluorouracil (5-FU), which is then transformed by cellular enzymes to potent pyrimidine antimetabolites (5-FdUMP, 5-FdUTP, and 5-FUTP). Three pathways are involved in the induced cell death: thymidylate synthase inhibition, formation of (5-FU) RNA and of (5-FU) DNA complexes.

The mustard prodrug CB1954 [5-(aziridin-1-yl)-2,4-dinitrobenzamide] is a weak monofunctional alkylator, but it can be efficiently activated by the rodent enzyme DT diaphorase into a potent DNA cross-linking agent. However, the human enzyme DT diaphorase shows a low reactivity with the prodrug, causing side effects. This problem was overcome when the E. coli enzyme nitroreductase (NTR) was found to reduce the CB1954 prodrug 90 times faster then the rodent DT diaphorase. The prodrug was converted to an alkylating agent which forms poorly repairable DNA crosslinks.

The oxazaphosphorine prodrug cyclophosphamide (CP) is activated by liver cytochrome P450 metabolism via a 4-hydroxylation reaction. The 4-hydroxy intermediate breaks down to form the bifunctional alkylating toxin phosphoramide mustard, which leads to DNA cross-links, G₂-M arrest and apoptosis in a cycle-independent fashion.

In the enzyme/prodrug systems described so far the prodrug is converted to an intermediate metabolite, which requires further catalysis by cellular enzymes to form the active drug. The decreased expression of or total lack of these enzymes in the target cells would lead to tumor resistance. The bacterial enzyme carboxypeptidase G2 (CPG2), which has no human analog, is able to cleave the glutamic acid moiety from the prodrug 4-[2-chloroethyl)(2-mesyloxyethyl)amino]benzoic acid without further catalytic requirements.

The reaction between the plant enzyme horseradish peroxidase (HRP) and the non-toxic plant hormone indole-3-acetic acid (IAA) has been analyzed in depth, but not yet completely elucidated. At neutral pH, IAA is oxidized by HRP-compound I to a radical cation, which undergoes scission of the exocyclic carbon-carbon bond to yield the carbon-centered skatolyl radical. In the presence of oxygen, the skatolyl radical rapidly forms a peroxyl radical, which then decays to a number of products, the major ones being indole-3-carbinol, oxindole-3-carbinol and 3-methylene-2-oxindole. In anoxic solution, decarboxylation of the radical cation can still take place and the carbon-centered radical preferentially reacts with hydrogen donors.

As can readily be seen, the prodrug/enzyme systems advantageously use an enzyme which is not produced by human cells to provide specificity. However, it can readily be seen by one of skill in the art that a human enzyme which is specifically produced in a particular organ or cell type could also be used to achieve this specificity, with the advantage that it would not be immunogenic.

Finally, heterogeneity could be circumvented by the application of a “cocktail” of conjugates constructed with the same enzyme and a variety of antibodies directed against different organ-associated antigens or different antigenic determinants of the same antigen.

### Administration of the Therapeutic Complexes

The therapeutic complexes of the present invention are said to be “substantially free of natural contaminants” if preparations which contain them are substantially free of materials with which these products are normally and naturally found.

The therapeutic complexes include antibodies, and biologically active fragments thereof, (whether polyclonal or monoclonal) which are capable of binding to tissue-specific luminally-expressed molecules. Antibodies may be produced either by an animal, or by tissue culture, or recombinant DNA means.

In providing a patient with the therapeutic complex, or when providing the therapeutic complex to a recipient patient, the dosage of administered agent will vary depending upon such factors as the patient's age, weight, height, sex, general medical condition, previous medical history, and the like. In addition, the dosage will vary depending on the therapeutic moiety and the desired effect of the therapeutic complex. As discussed below, the therapeutically effective dose can be lowered if the therapeutic complex is administered in combination with a second therapy or additional therapeutic complexes. As used herein, one compound is said to be additionally administered with a second compound when the administration of the two compounds is in such proximity of time that both compounds can be detected at the same time in the patient's serum.

The therapeutic complex may be injected via arteries, arterioles, capillaries, sinuses, lymphatic ducts, epithelial cell perfusable spaces or the like. When administering the therapeutic complex by injection, the administration may be by continuous infusion, or by single or multiple boluses.

The therapeutic complex may be administered either alone or in combination with one or more additional immunosuppressive agents (especially to a recipient of an organ or tissue transplant), antibiotic agents, chemotherapeutic agents, or other pharmaceutical agents, depending on the therapeutic result which is desired. The administration of such compound(s) may be for either a “prophylactic” or a “therapeutic” purpose.

A composition is said to be “pharmacologically acceptable” if its administration can be tolerated by a recipient patient. Such an agent is said to be administered in a “therapeutically effective amount” if the amount administered is physiologically significant. A typical range is 0.1 μg to 500 mg/kg of therapeutic complex per the amount of the patients weight. One or multiple doses of the therapeutic complex may be given over a period of hours, days, weeks, or months as the conditions suggest. An agent is physiologically significant if its presence results in a detectable change in the physiology of a recipient patient. The term “pharmaceutically effective amount” refers to an amount effective in treating or ameliorating an IL-1 mediated disease in a patient. The term “pharmaceutically acceptable carrier, adjuvant, or excipient” refers to a non-toxic carrier, adjuvant, or excipient that may be administered to a patient, together with a compound of the preferred embodiment, and which does not destroy the pharmacological activity thereof. The term “pharmaceutically acceptable derivative” means any pharmaceutically acceptable salt, ester, or salt of such ester, of a compound of the preferred embodiments or any other compound which, upon administration to a recipient, is capable of providing (directly or indirectly) a compound of the preferred embodiment. Pharmaceutical compositions of this invention comprise any of the compounds of the present invention, and pharmaceutically acceptable salts thereof, with any acceptable carrier, adjuvant, excipient, or vehicle.

The therapeutic complex of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (18^{th} ed., Gennaro, Ed., Mack, Easton Pa. (1990)). In order to form a pharmaceutically acceptable composition suitable for effective administration, such compositions will contain an effective amount of the therapeutic complex, together with a suitable amount of carrier vehicle.

Additional pharmaceutical methods may be employed to control the duration of action. Controlled release preparations may be achieved through the use of polymers to complex or absorb the therapeutic complex. Alternatively, it is possible to entrap the therapeutic complex in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1990).

A number of embodiments have been described. Nevertheless, it will be understood that various modifications may be made without departing from the spirit and scope of the invention. For example, a variety of cleavable chemical moieties, surface molecules, and therapeutic moieties can be used in the instant methods Accordingly, other embodiments are within the scope of the invention.

Having now generally described the invention, the following examples are offered to illustrate, but not to limit the claimed invention.

### EXAMPLES

The following tissue-specific molecules were identified and isolated using the method of Roben et al., U.S. Pat. No. 09/528,742, filed Mar. 20, 2000 (herein incorporated by reference). The method used a cell membrane impermeable reagent which nonspecifically binds to luminal molecules via a chemical reaction. The reagent comprised a first reactive domain which binds to the molecules in the lumen nonspecifically and a second biotin-comprising domain, linked by a cleavable chemical moiety that will not cleave under in vivo conditions, but can be induced to cleave under defined conditions. The binding reagent was injected via arteries, arterioles, capillaries, sinuses, lymphatic ducts, epithelial line perfusable spaces or the like. The reagent bound to the lumen specific molecules. The tissue or organ was homogenized, and cell debris removed. All of the molecules which bound the reagent were isolated from the organ using affinity chromatography which bound the biotin-comprising domain (i.e., a streptavidin bead). Then, the lumen-exposed molecules which were “tagged” with the reagent were eluted by cleaving the reagent under “mild conditions” (mild reducing, non-denaturing conditions). Thus, the tissue-specific molecules were eluted and purified on PAGE. An organ specific molecule was identified as such and isolated from the PAGE and partially sequenced to determine its identity. Then histology, Western blots and/or in vivo localizations were performed to confirm the tissue specificity of the isolated polypeptide.

In Example 1, an endothelial specific protein was identified as such and an antibody specific to the protein was used to show that when injected into the tail vein of a rat, the antibody would specifically bind to brain. Example 1 shows that an antibody to a tissue-specific endothelial protein can be used to target a specific organ and that that antibody can be coupled to a therapeutic moiety and will direct that therapeutic moiety to the specific organ, where it can exert its effect.

### Example 1

### Localization of the Therapeutic Moiety to Tissue Using a Brain-Specific, Luminally Expressed Protein, CD71

CD71, or transferrin receptor, is known to be exposed on the luminal surface of the endothelium in only one tissue: the brain. This molecule was found to exist only in the brain preparation and not in any other tissues using the instant methods, confirming the ability of the method to identify tissue specific endothelial proteins.

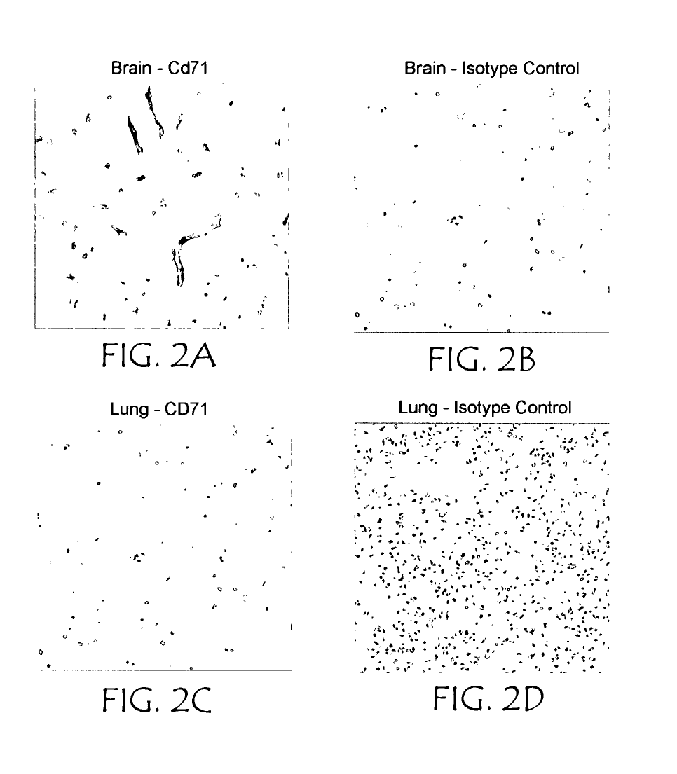

To demonstrate the ability to use the tissue-specific endothelial expression of a protein to selectively deliver an agent to a particular tissue, an antibody to the rat CD71 was used (BD Pharmingen, San Diego, Calif., catalog number 22191). CD71 is a luminally exposed endothelial protein specific to the brain. The rat amino acid and nucleotide sequences are Genbank Accession Nos. AAA42273 and M58040 (SEQ ID NOs:26 and 27), the human amino acid and nucleotide sequences are Genbank Accession Nos. AAH01188 and BC001188 (SEQ ID NOs:28 and 29). The antibody was injected into the tail vein of a rat. Another antibody with a similar isotype but different specificity was injected into another rat as a control. The antibody used as an isotype control was an anti-albumin antibody (IgG2) that was produced by Target Protein Technologies. After 30 minutes, the rats were sacrificed and tissue sections were made from a number of organs from each rat. Each tissue was then analyzed by immunohistochemistry for the presence of the antibodies. FIGS. 2A-D show the immunohistochemistry of tissue sections from a rat which was injected with either CD71 or a control antibody. FIG. 2A is brain from a rat injected with CD71, FIG. 2B is brain from a rat injected with the control antibody, FIG. 2C is lung from a rat injected with CD71, FIG. 2D is lung from a rat injected with the control antibody. These results demonstrate that the anti-CD71 antibody localized to the capillaries of the brain, and to no other tissue. This is particularly advantageous in that it is often difficult to find therapeutics which can cross the blood-brain barrier.

In a follow-up experiment, a toxin was coupled to the anti-CD71 antibody. The toxin used was the Ricin A chain (Sigma, Catalog number L9514). This was coupled to the antibody by adding a biotin with a disulfide-containing linker (Pierce, catalog number 21331) to both the ricin and the antibody. The two were then coupled by the addition of Nuetravidin (Pierce, catalog number 31000) which bound both biotins, thus forming a complex of the ricin and antibody. The in vivo localization experiment was repeated using the toxin-antibody complex. In this case, the antibody not only facilitated the localization of the toxin to the vasculature of the brain, but presumably also its entry into the tissue via transcytosis. Once in the tissue, the toxin elicited an inflammatory response in the brain, a reaction typically seen for any toxin introduced into the brain. No inflammatory response was seen in any other sectioned tissue.

A human CD71-specific antibody is available from BD Pharmingen and usable for the production of a human therapeutic complex.

In Examples 2-6, a number of other tissue-specific luminally expressed proteins were identified and used to produce therapeutic complexes.

### Example 2

### Identification and Sequencing of Rat Dipeptidyl peptidase IV

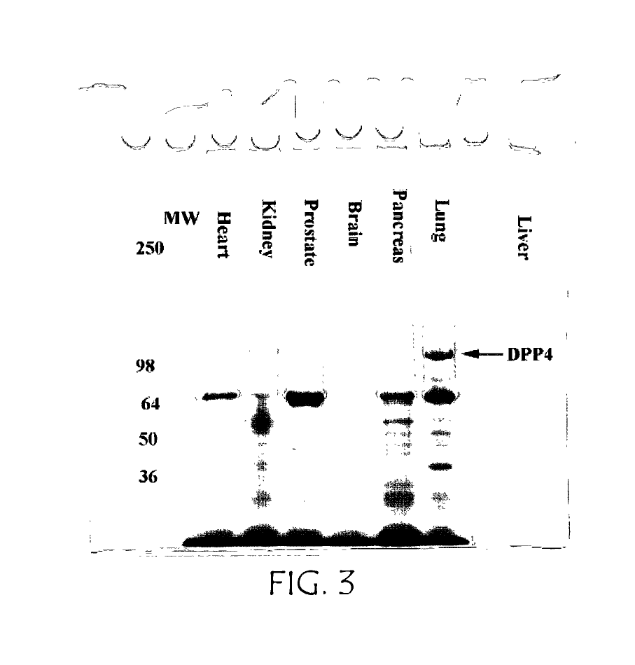

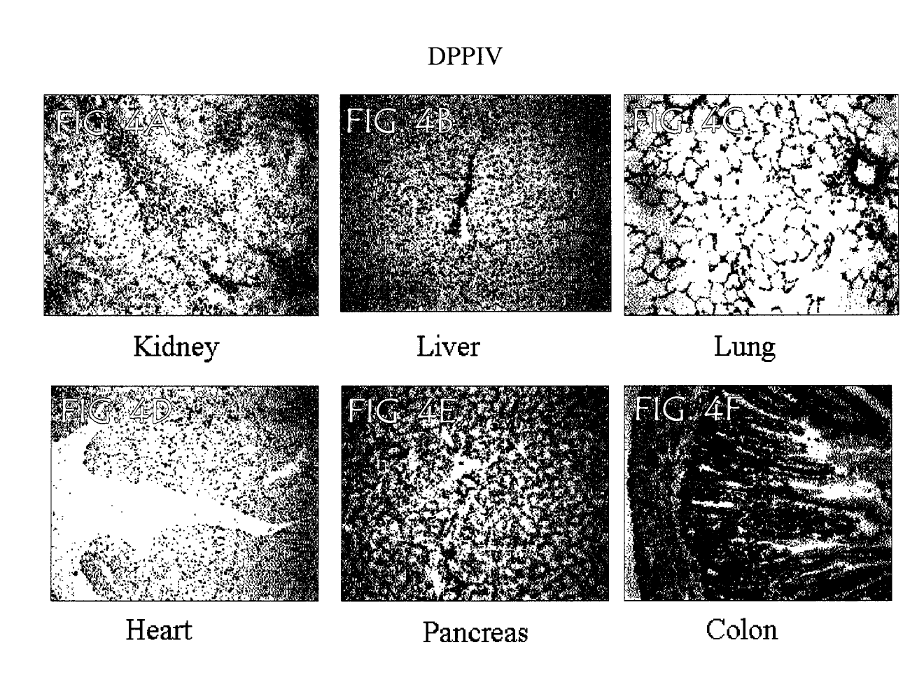

The luminal proteins of the vasculature of an entire rat were labeled with biotin. Then the organs were removed individually and the labeled proteins were isolated as described in Roben et al., U.S. Pat. No. 09/528,742, filed Mar. 20, 2000. The labeled proteins that were isolated from the homogenized lung were subjected to polyacrylamide gel electrophoresis and a protein (labeled DPP-4) which was specific to lung and kidney (FIG. 3), but predominately lung was identified. A peptide was sequenced corresponding to the sequence, FRPAE (SEQ ID NO:3) and the protein was identified as rat liver dipeptidyl peptidase IV, Genbank Accession Number P14740 (nucleotide sequence Genbank Accession Number NM_{—}012789). The full-length protein sequence corresponds to SEQ ID NO:4 and the nucleotide sequence is SEQ ID NO:5. The protein sequence is encoded by nucleotides 89-2392 of NM_{—}012789. The human sequences correspond to SEQ ID NOS:6 and 7. Genbank Accession Number NM_{—}001935 is SEQ ID NO:6 and the coding region of the mRNA is from nt 76 to 2376 (SEQ ID NO:7). Previous studies suggest that the rat liver dipeptidyl peptidase IV has a membrane anchoring region consisting of its amino terminus. (Ogata et al., J. Biol Chem 264(6):3596-601 (1989) ). A monoclonal antibody specific to rat dipeptidyl peptidase IV (BD Pharmingen, San Diego, Calif. Catalog number 22811) was injected into the tail vein of a rat (about 0.1 to 100 mg/ml). The tissue from various organs was treated using immunohistochemistry and the antibody to DPP-4 was shown to localize to lung and kidney (see FIG. 4). In FIG. 4 panel a. kidney, panel b. liver, panel c. lung, panel d. heart, panel e. pancreas, and panel f. colon.

An antibody to human DPP-4 is available for use in producing the therapeutic complex of the invention (BD Pharmingen, San Diego, Calif.).

### Example 3

### Identification and Sequencing of Carbonic Anhydrase IV

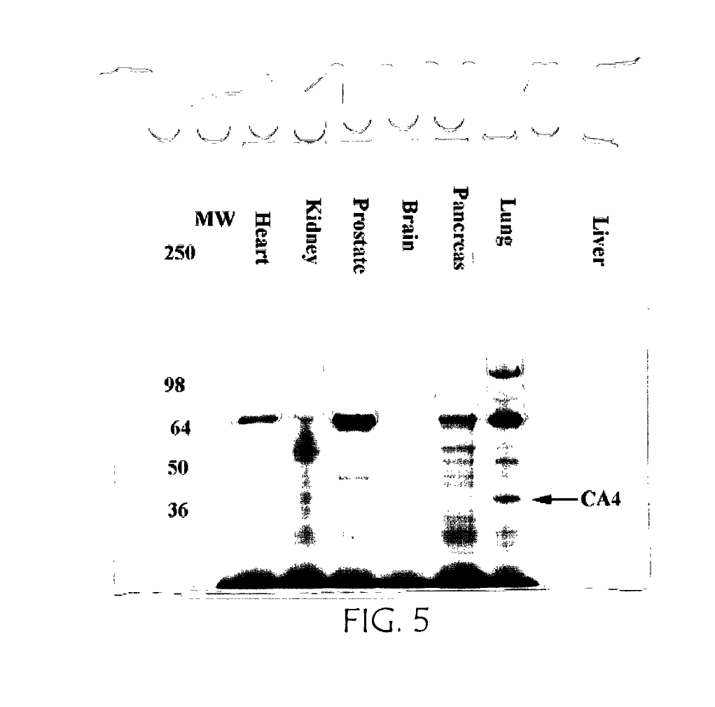

The luminal proteins of the vasculature of an entire rat were labeled with biotin. Then the organs were removed individually and the labeled proteins were isolated as described in Roben et al., U.S. Pat. No. 09/528,742, filed Mar. 20, 2000. The labeled proteins that were isolated from the homogenized lung were subjected to polyacrylamide gel electrophoresis showed a protein (labeled CA-4) which was subsequently shown to be specific to lung and heart (FIG. 5). A peptide was sequenced corresponding to the sequence, DSHWCYEIQ (SEQ ID NO: 8) and identified as rat Carbonic Anhydrase IV, Genbank Accession Number NM_{—}019174. The full-length protein sequence corresponds to SEQ ID NO: 9 and the nucleotide sequence is SEQ ID NO:10. The human sequence corresponds to SEQ ID NOS: 11 and 12, Genbank Accession Number NM_{—}000717. Previous studies suggest that carbonic anhydrase IV shows developmental regulation and cell-specific expression in the capillary endothelium (Fleming et al., Am J. Physiol, (1993) 265 (6 Pt 1):L627-35).

### Example 4

### Identification and Sequencing of Zvmogen Granule 16 Protein (ZG16-p)

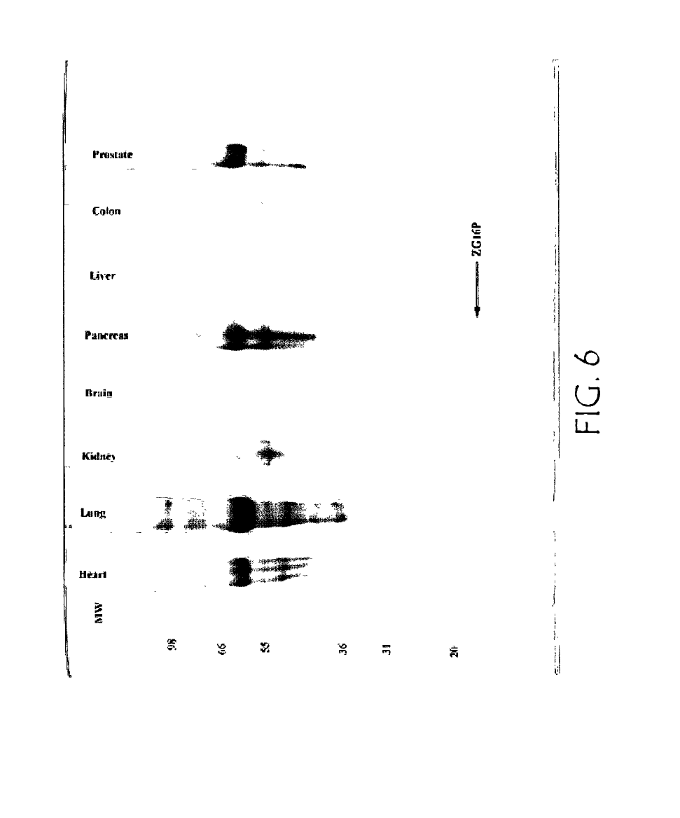

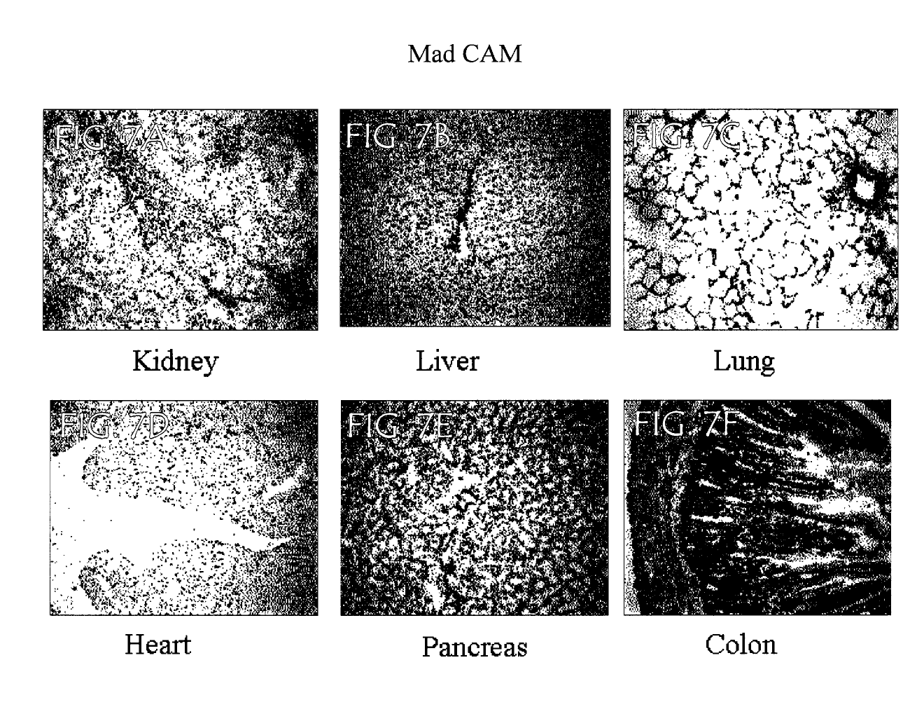

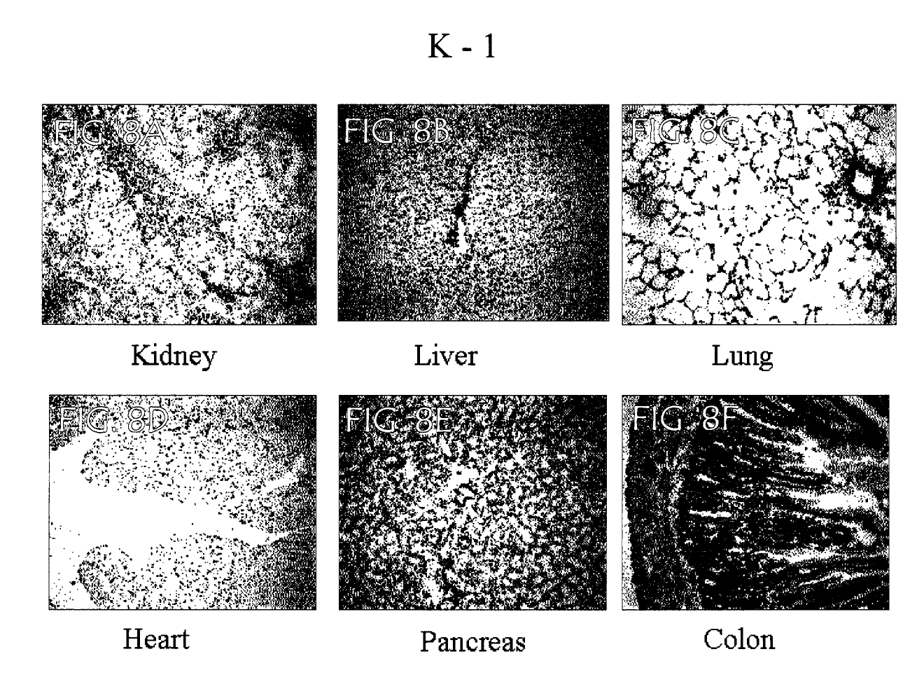

The luminal proteins of the vasculature of an entire rat were labeled with biotin. Then the organs were removed individually and the labeled proteins were isolated as described in Roben et al., U.S. Pat. No. 09/528,742, filed Mar. 20, 2000. The labeled proteins that were isolated from the homogenized pancreas were subjected to polyacrylamide gel electrophoresis and a protein (labeled ZG16P) which was subsequently shown to be specific to pancreas and gut (see FIG. 6), but predominately pancreas was identified. The peptide was sequenced and the sequence NSIQSRSSSY, SEQ ID NO:13 was obtained and identified as rat ZG16-p, Genbank Accession Number Z30584. The full-length protein sequence corresponds to SEQ ID NO:14 and the nucleotide sequence is SEQ ID NO:15. The human sequence corresponds to SEQ ID NOS:16 and 17, Genbank accession No. AF264625. Previous studies suggest that ZG16-p is located in zymogen granules of rat pancreas and goblet cells of the gut. (Cronshagen and Kern, Eur J. Cell Biology 65: 366-377, 1994).

### Example 5

### Identification and Sequencing of Rat MAdCAM

A monoclonal antibody was purchased from BD Pharmingen (catalog number 22861) and about 0.1 to 100 mg/ml were injected into the tail vein of a rat. The tissue from various organs was treated using immunohistochemistry and the antibody to MAdCAM (MadCam-1) was shown to localize to pancreas and colon (FIG. 7). In FIG. 7 panel a. kidney, panel b. liver, panel c. lung, panel d. heart, panel e. pancreas, and panel f. colon. Rat MadCam-1, Genbank Accession Number D87840 corresponds to protein sequence, SEQ ID NO:18 and the nucleotide sequence is SEQ ID NO:19. The human sequence corresponds to SEQ ID NOS:20 and 21, Genbank Accession Number U82483. A human MadCam-1 antibody is available from BD Pharmingen (San Diego, Calif.) to produce the therapeutic complex of the invention for human use.

### Example 6

### Identification of CD90

An antibody to the rat CD90 was purchased (BD Pharmingen, San Diego, Calif., catalog number 22211 D) and about 0.1 to 100 mg/ml was injected into the tail vein of a rat. The tissue from various organs was treated using immunohistochemistry and the antibody to Thy-1 was shown to localize to kidney (FIG. 8). In FIG. 8 panel a. kidney, panel b. liver, c. lung, d. heart, e. pancreas, and f. colon. Rat Thy-1, Genbank Accession Number NP036805 corresponds to protein sequence SEQ ID NO:30 and Genbank Accession Number NM 012673 to nucleotide sequence SEQ ID NO:31. Human Thy-1, Genbank Accession Number XP006076 corresponds to protein sequence SEQ ID NO:32 and Genbank Accession Number XM 006076 to nucleotide sequence SEQ ID NO:33 (see also Genbank Accession Number AF 261093). A mouse anti-rat Thy-1 antibody is available from Pharmingen Intl. and was used for immunohistochemistry at a concentration of 0.5 to 5 μg/ml to produce the therapeutic complex of the preferred embodiment for human use.

### Example 7

### Identification and Sequencing of an Albumin Fragment

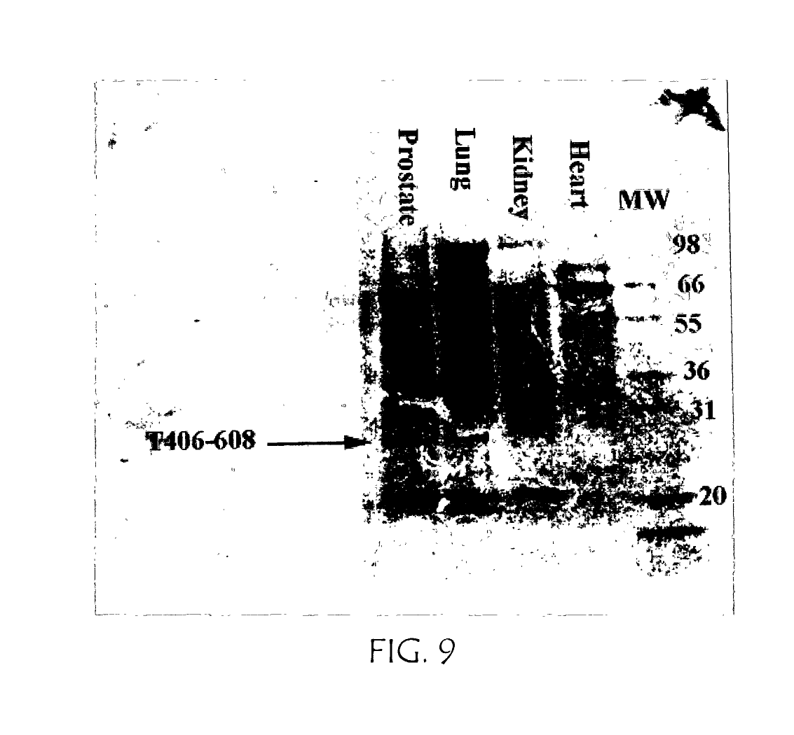

The luminal proteins of the vasculature of an entire rat were labeled with biotin. Then the organs were removed individually and the labeled proteins were isolated as described in Roben et al., U.S. Pat. No. 09/528,742, filed Mar. 20, 2000. The labeled proteins that were isolated from the homogenized prostate were subjected to polyacrylamide gel electrophoresis which identified a protein labeled T436-608 (FIG. 9). The protein was partially sequenced and identified as a fragment of Albumin TQKAPQVST (SEQ ID NO:22). In addition, sequencing showed that the prostate-specific form was a fragment in which translation was terminated early, corresponding to amino acids 436 to 608 of the full-length albumin protein (SEQ ID NO:23). The Albumin fragment has been identified by others as a vasoactive fragment (Histamine release induced by proteolytic digests of human serum albumin: Isolation and structure of an active peptide from pepsin treatment, Sugiyama K, Ogino T, Ogata K, Jpn J. Pharmacol, 1989 Feb., 49(2): 165-71). The rat protein sequence is SEQ ID NO: 24 (Genbank Accession No. P02770). The human counterpart is shown as SEQ ID NO:25, Genbank accession No. P02768.

In Example 8, the in vivo distribution of the luminally expressed target proteins isolated and identified in the previous Examples is described.

### Example 8

### Biodistribution of DPP-4, MadCam-1, CD90 and CA-4

The following example describes the use of specific labeled antibody ligands to visualize the biodistribution of several of the luminally expressed target proteins that were identified in previous Examples. Specifically, 50 μl of a 1 μg/μl solution of an antibody specific for DPP-4, MadCam-1, CD90 or CA-4 was injected into the tail veins of a group of Sprauge-Dawley rats. The antibody was allowed to circulate for about thirty minutes after which time the animals were sacrificed and their organs removed. Small cubes of brain, heart, lungs, liver, pancreas, colon and kidneys were excised, placed in embedding medium and immediately frozen. The frozen cubes were kept on dry ice until they were sectioned. The tissues were sectioned in 6 μm slices using a cryostat, air-dried overnight and fixed in acetone for two minutes. The fixed tissue sections were incubated with Cy3-labeled secondary antibodies, rinsed then mounted for subsequent image capture. At least three independent experiments were performed for each luminally expressed target protein.

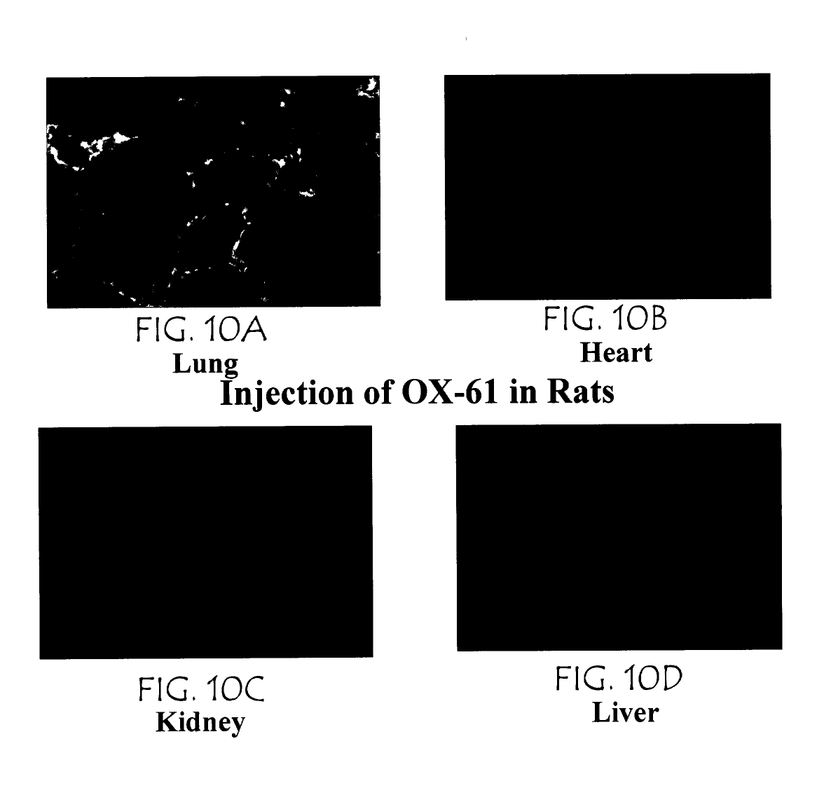

Using the above-described method, the biodistribution of DPP-4 was verified by using OX-61 (Pharmingen), a mouse monoclonal antibody that is specific for the luminally expressed target protein DPP-4. FIG. 10A shows strong fluorescent staining, which indicates that DPP-4 is present in the lung. Additional weak staining was observed in the glomeruli of the kidney (FIG. 10B); however, DPP-4 was not significantly found in any of the other tissues that were examined (FIGS. 10C-D). These results indicate that DPP-4 is primarily localized to the endothelium of the lung.

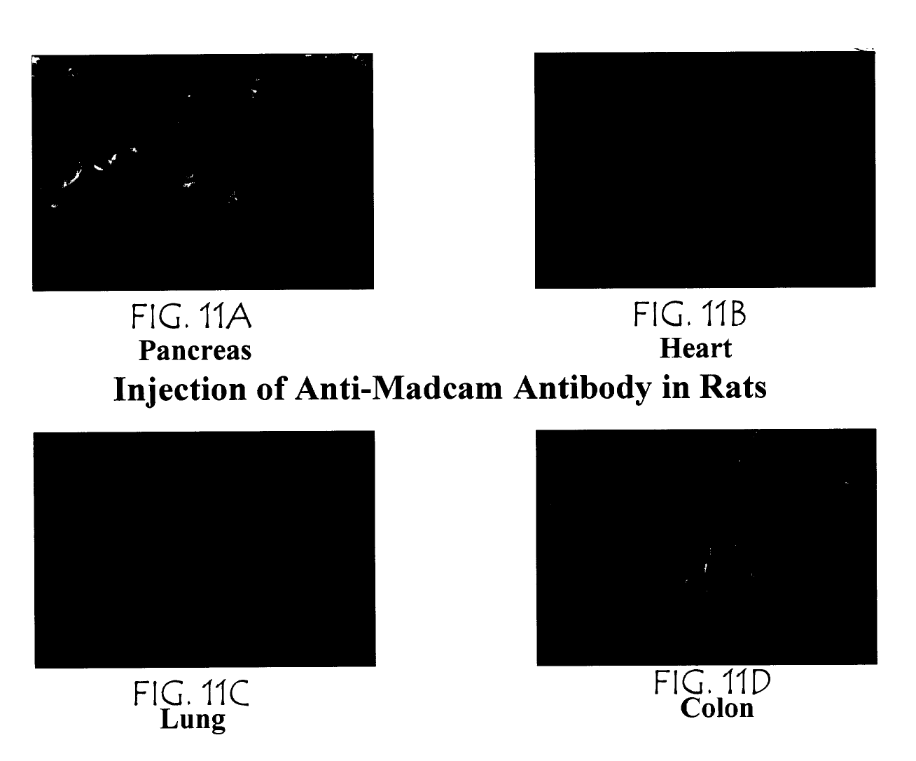

The biodistribution of MadCam-1 was also verified by using the above methods. Specifically, OST-2 (Pharmingen), a mouse monoclonal antibody that recognizes rat MadCam-1, was used. FIGS. 11A and 11D show that fluorescence was observed in both pancreas and the colon. Additional staining was observed in the small intestine. In contrast, very little fluorescence was observed in the other tissues that were examined (e.g. FIGS. 11B-C). These results indicate that MadCam-1 is localized to certain tissues that comprise the gastrointestinal (GI) tract.

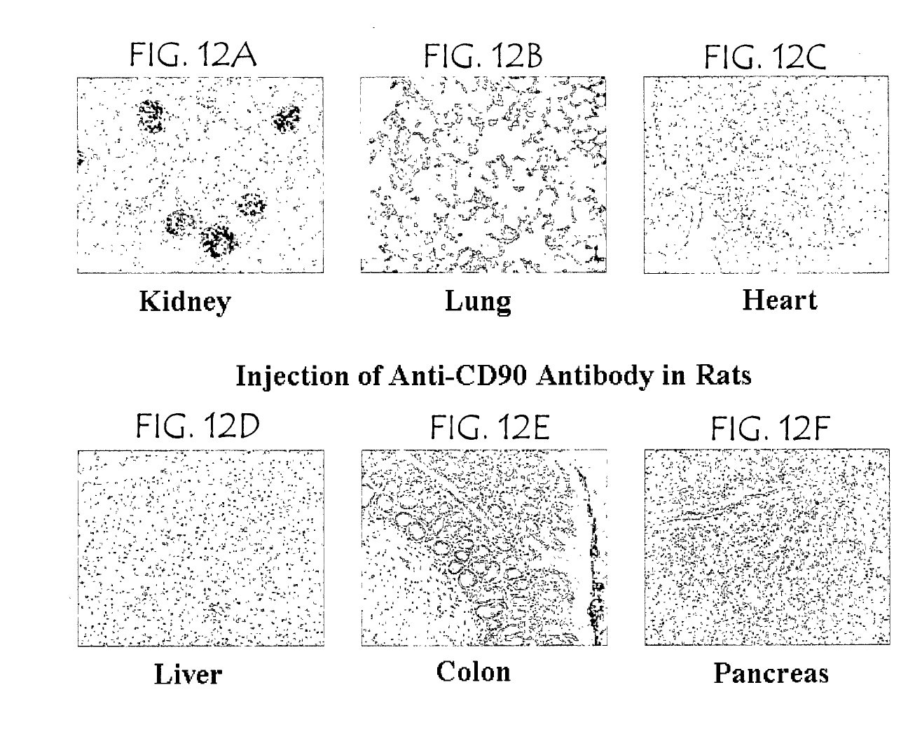

The biodistribution of CD90 was verified by administering OX-7 (Pharmingen), a mouse monoclonal antibody that specifically recognizes rat CD90. FIG. 12A shows the fluorescent staining that was observed in the kidney. No staining was detected in any of the other tissues that were examined (FIGS. 12B-F). These results indicate that CD90 is localized only in the kidney.

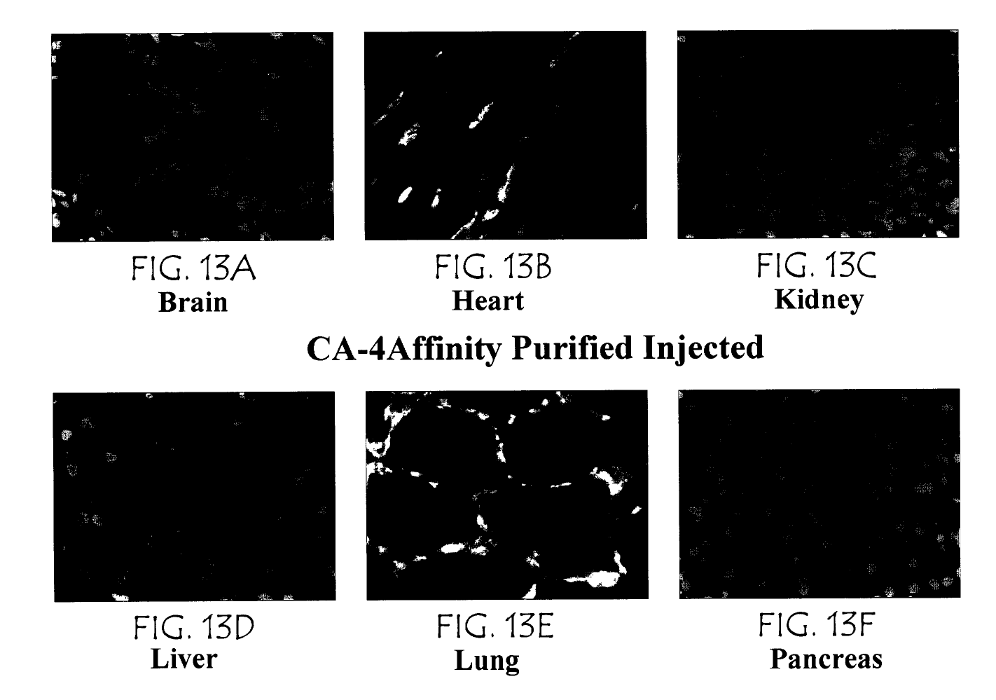

To determine the biodistribution of CA-4, a rabbit polyclonal antibody that recognizes rat CA-4 was generated using methods well known in the art. Using the above-described administration and histology procedures, this polyclonal antibody was then used to determine the localization of CA-4. Strong staining was observed in both the heart (FIG. 13B) and the lung (FIG. 13E) indicating the presence of CA-4. No staining was observed in brain (FIG. 13A), kidney (FIG. 13C), liver (FIG. 13D) or pancreas (FIG. 13F). A monoclonal antibody that is specific for CA-4 was also found to bind specifically to the heart and lung but not to other tissues. These results indicate that CA-4 is specifically localized to the heart and lung.

In Examples 9-13, the characteristics of ligand binding to specific luminally expressed proteins in target tissues is described.

### Example 9

### Relationship Between Ligand Dose and Specificity of Localization to Target Tissues

The following example describes the specificity of localization of antibody ligands to target tissues in relation to the amount of antibody that is administered. Specifically, mouse monoclonal antibodies specific to DPP-4, MadCam-1 or CD90 were administered to Sprague-Dawley rats via tail-vein injection. Each of the rats received either 5 μg, 20 μg, 50 μg or 100 μg of one of the above antibodies. Following the injection, the antibody was allowed to circulate for thirty minutes after which time the animals were sacrificed and their organs were removed. The organs were then processed for immunohistochemistry as described in Example 8.

Using the above-described method, the OX-61 monoclonal antibody was used to determine the relationship between the amount of antibody ligand administered and its specificity for the luminally expressed target protein DPP-4 in the lung. When administered to rats in doses of 5 to 50 μg, OX-61 displayed a high degree of specificity to the lung. However, when 100 μg or more was injected in a single dose, the OX-61 antibody began to appear in the kidneys. These results are consistent with the bioavailability data for DPP-4 presented in Example 8.

The monoclonal antibody, OST-2, was used in similar studies to determine the effect of dosage on its specificity for MadCam-1 in the pancreas and other GI organs. When administered in 5 μg, 20 μg, 50 μg and 100 μg doses, OST-2 remained specific for the pancreas and other tissues of the GI tract. These results seem to indicate that MadCam-1 specificity is limited to the GI tract irrespective of the dose that is administered.

The monoclonal antibody, OX-7, was used to determine the effect of dosage on its specificity for CD90 in the kidney. From doses of 5 to 50 μg, OX-7 displayed complete specificity for the kidney. However, at 100 μg, a small amount of OX-7 began to appear in the lung and liver. Although some OX-7 was detectable in lung and liver at high antibody concentrations, the amount of OX-7 present in the lung and liver was far less than the amount of OX-7 which appeared in the kidneys.

### Example 10

### Characterization of Ligand Binding to Target Tissues Over Time

The following example describes the binding of antibody ligands to specific target tissues throughout time. Specifically, mouse monoclonal antibodies specific to DPP-4, MadCam-1 or CD90 were administered to Sprague-Dawley rats via tail-vein injection. Each of the rats received a 50 μg dose of a single antibody which was allowed to circulate for time periods ranging from 5 minutes to 48 hours. Following the period of antibody circulation, the animals were sacrificed and their organs were processed for immunohistochemistry as described in Example 8.

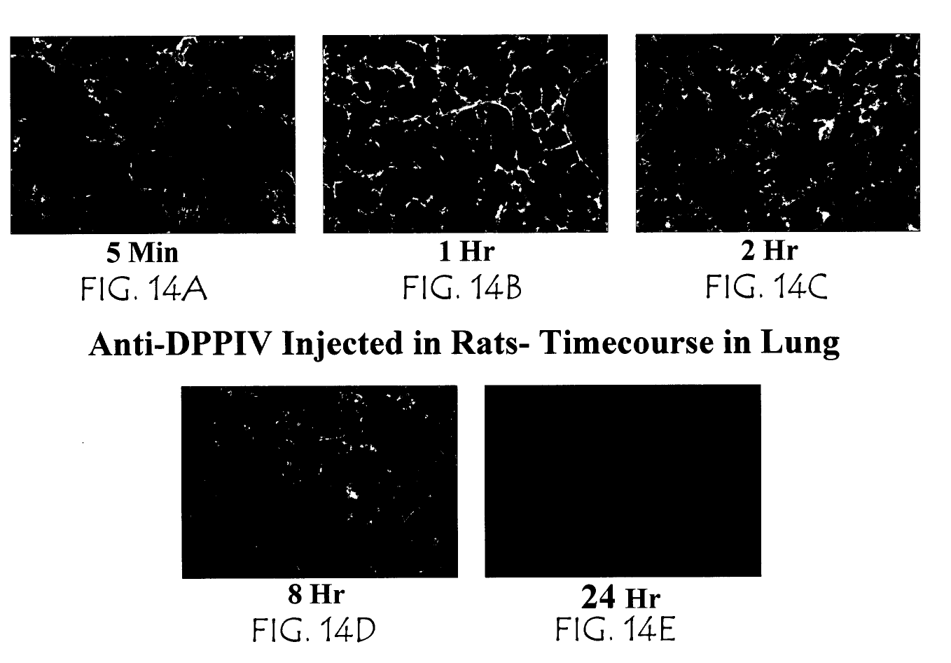

Using the above-described method, a profile of the binding of the OX-61 monoclonal antibody to DPP-4 in the vasculature of the lung was determined with respect to time. FIGS. 14A-E show the amount of OX-61 that localized to the lung during time periods ranging from 5 minutes to 24 hours after intravenous injection. Specifically, OX-61 was detected in the lung in as little as 5 minutes subsequent to administration (FIG. 14A). Similar amounts of this antibody were detected in the lung for at least eight hours after administration (FIGS. 14B-D). At 24 hours subsequent to the administration, however, the amount of OX-61 detectable in the lung had significantly decreased (FIG. 14E).

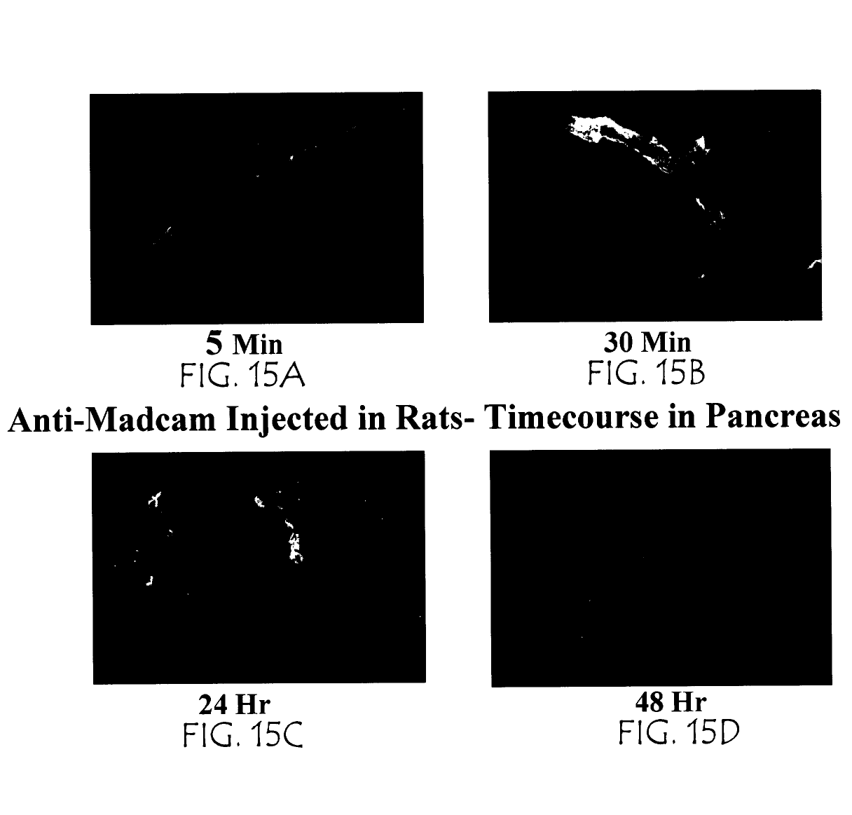

A profile with respect to time was established for the binding of the OST-2 monoclonal antibody to MadCam-1 in the vasculature of the pancreas. FIGS. 15A-D show the amount of OST-2 that was detected in the pancreas during time periods ranging from 5 minutes to 48 hours. Specifically, OST-2 was detected in the pancreas within 5 minutes subsequent to administration (FIG. 15A). In addition, similar amounts of this antibody were detected in the pancreas after 30 minutes, 24 hours and even 48 hours post injection (FIGS. 15A-D).

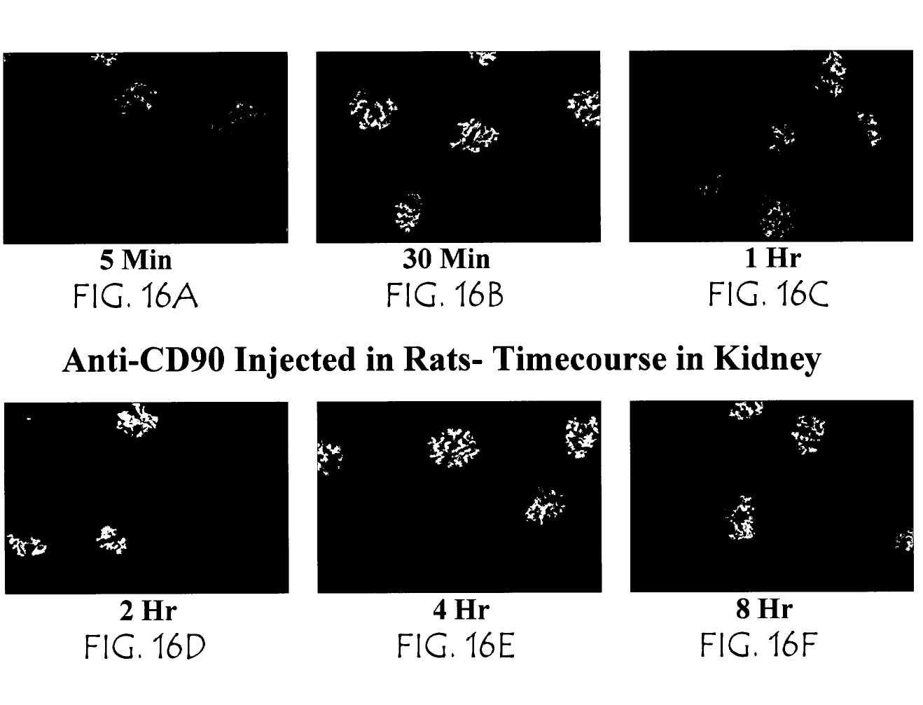

A profile with respect to time was also established for the binding of the OX-7 monoclonal antibody to the luminally expressed target protein CD90 in the vasculature of the kidney. FIGS. 16A-F show the amount of OX-7 that had localized to the kidney during time periods ranging from 5 minutes to 8 hours. Specifically, OX-7 was detected in the kidney in as little as 5 minutes subsequent to administration (FIG. 16A). Similar amounts of this antibody were detected in the kidney for at least eight hours after its administration (FIGS. 16B-F).

### Example 11

### Quantification of Antibody Ligand Bound to Target Tissues by Time-Resolved Fluorescence

The following example describes quantitative analyses of antibody ligands localized to luminally expressed target proteins in various target tissues. Specifically, antibodies specific for DPP-4, MadCam-1 or CA-4 were each labeled with approximately three molecules of Europium per antibody molecule using a europium-DTPA labeling kit (Perkin Elmer, Cat# AD0021) according to manufacturer's instructions. Additionally, monoclonal antibodies specific for influenza virus (IgG2a and IgG1 isotypes) were also labeled for use as isotype controls. After labeling, the antibody/Europium conjugates were injected into the tail veins of Sprauge-Dawley rats at doses of 5 μg, 20 μg and 50 μg. For each dosage level, the antibodies were allowed to circulate for either 30 minutes, 6 hours or 24 hours. At least three independent experiments were performed for each dose and time point combination.

At the end of each time period, the rats were sacrificed and their organs were processed for fluorescence analysis. Organs that were examined typically included, kidney, lung, liver, brain, pancreas, small intestine, large intestine (colon), stomach and heart. Excised organs were first homogenized in ten volumes of enhance solution (Perkin Elmer, Cat# 400-0010) then incubated overnight at 4° C. One percent of the resulting solution was then diluted 1:40 into fresh enhance solution, rotated for 30 minutes at room temperature and centrifuged at 1500 g for 10 minutes. The resulting solution was placed in a fluorimeter and the signal intensity was measured three times.

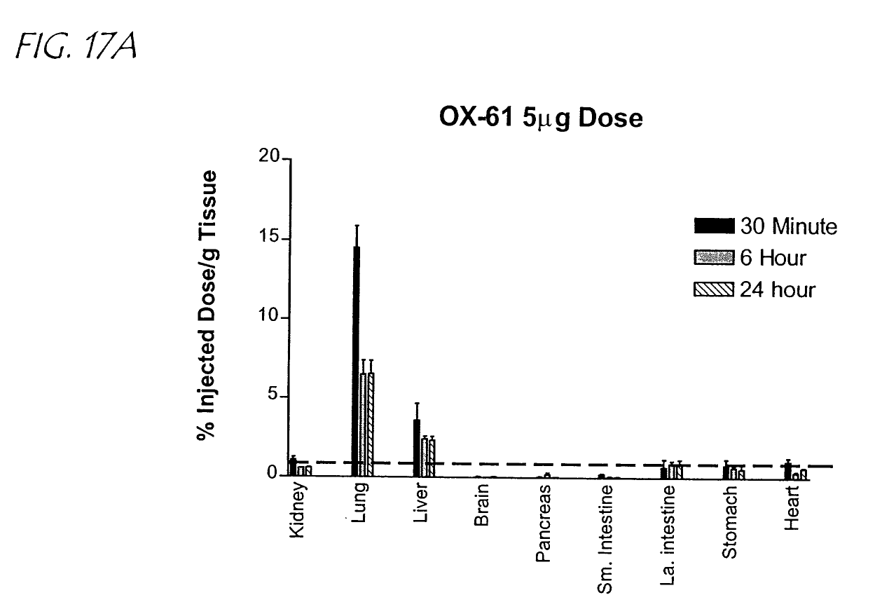

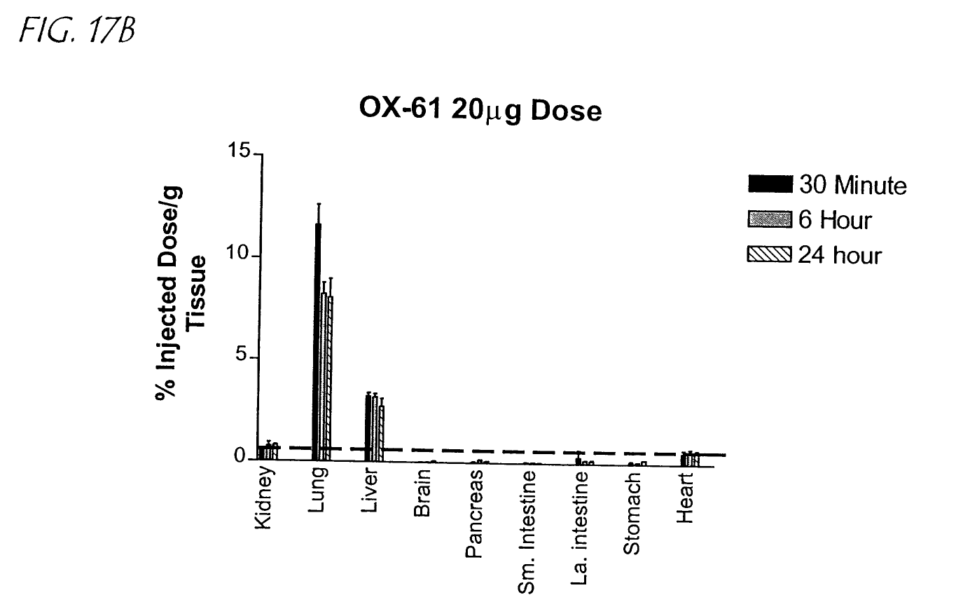

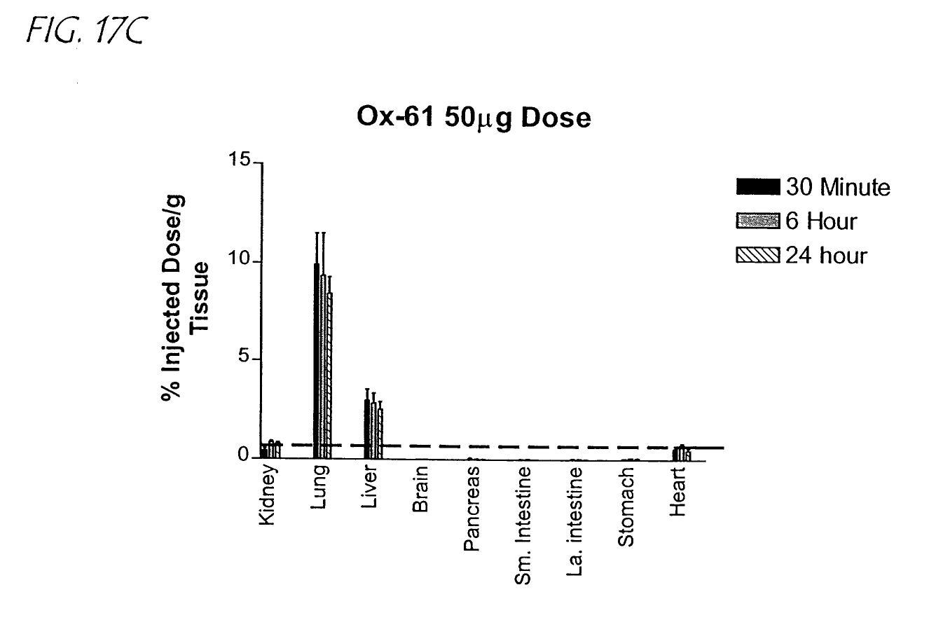

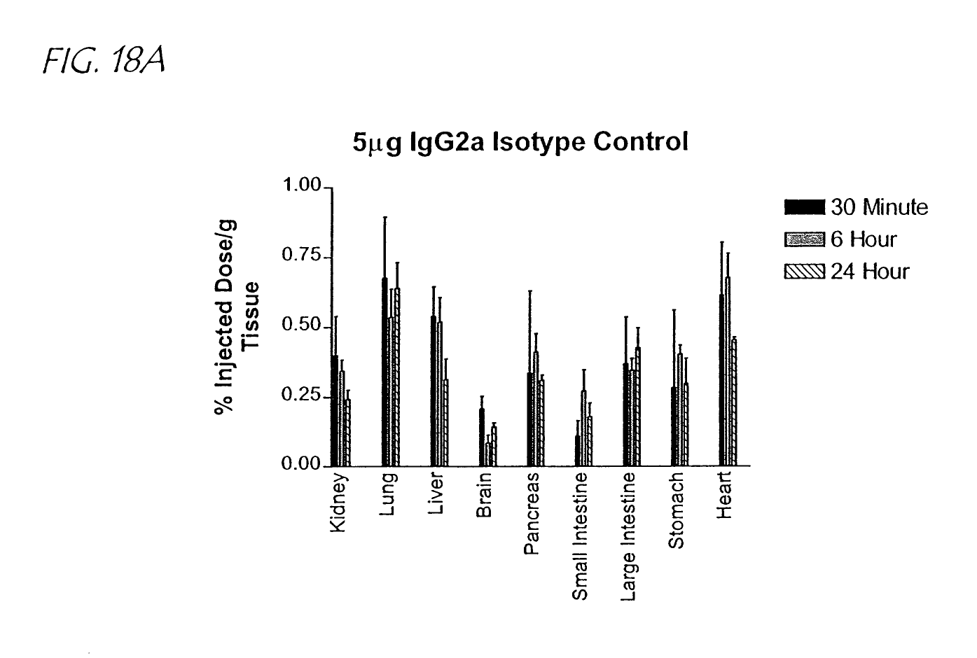

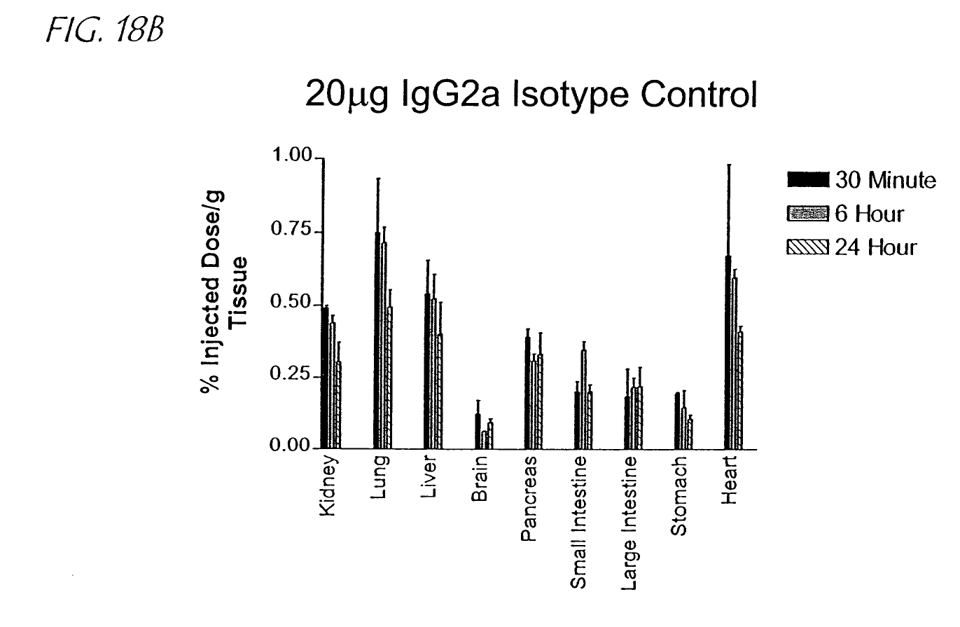

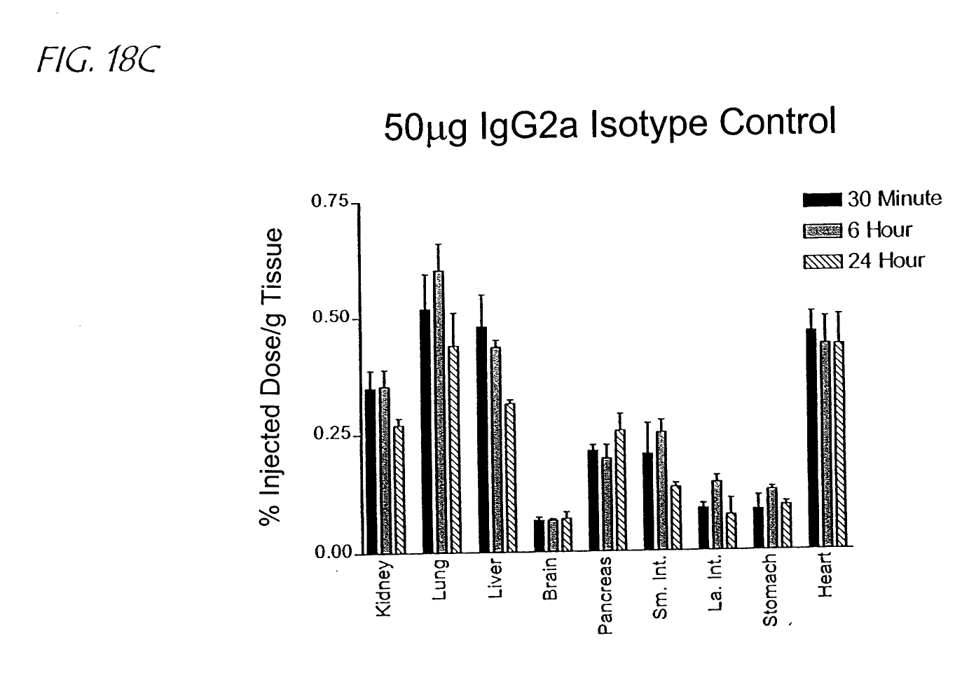

Using the above-described method, the amount of OX-61 (anti-DPP-4) antibody localized in each tissue type was determined at specific time points for each antibody dose that was administered.. IgG2a isotype anti-influenza monoclonal antibodies were used as a control for background fluorescence. FIGS. 17A-C show the weight percent of OX-61 that was present in each tissue at each time point tested for each dosage level. Specifically, FIG. 17A shows that approximately 15% of the total 5 μg dose localized in the lungs after 30 minutes. By 6 hours, the level had fallen to about 7% but then remained constant up to the 24 hour timepoint. For the most part, the amount of OX-61 localized to other tissues was less than 0.75% of the dose weight, which corresponds to the maximum levels of anti-influenza control antibody that localized to each tissue type (FIG. 18A-C and FIG. 17A, dashed line). One exception was the slightly increased localization to the liver.

Results similar to those obtained for the 5 μg doses were also obtained for the 20 and 50 μg doses (FIGS. 17A-C, respectively). With respect to levels of OX-61 in the lung, it should be noted that as the initial dose increased, the percentage loss of OX-61 localized to the lung over time was reduced (FIGS. 17A-C). Taken together, these results indicate that high levels of OX-61 localize specifically to the lung and the levels of antibody remain high over a long period of time. Such high levels of localization will likely result in a significant improvement in the therapeutic index of any lung-acting drug delivered using this antibody ligand.

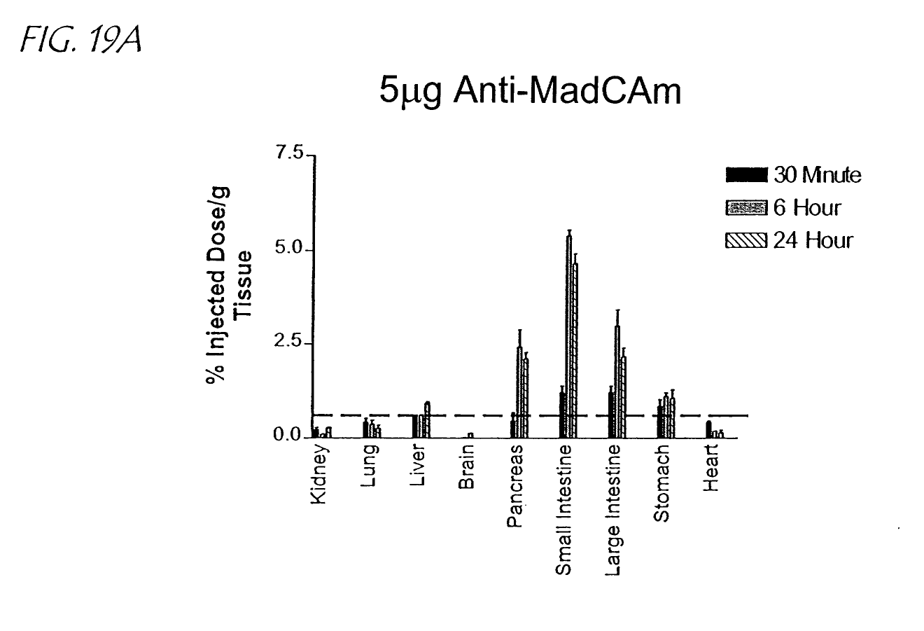

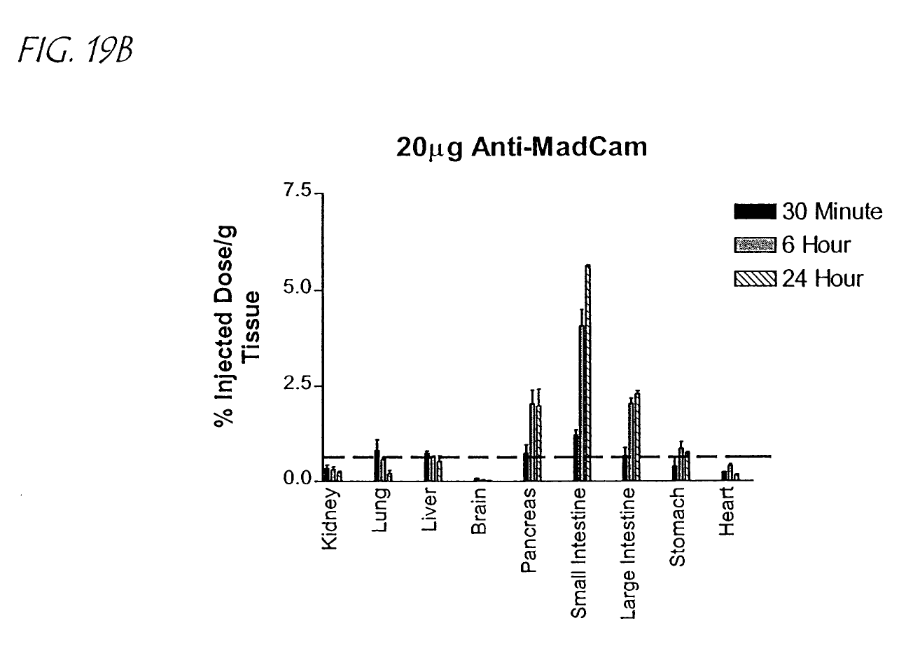

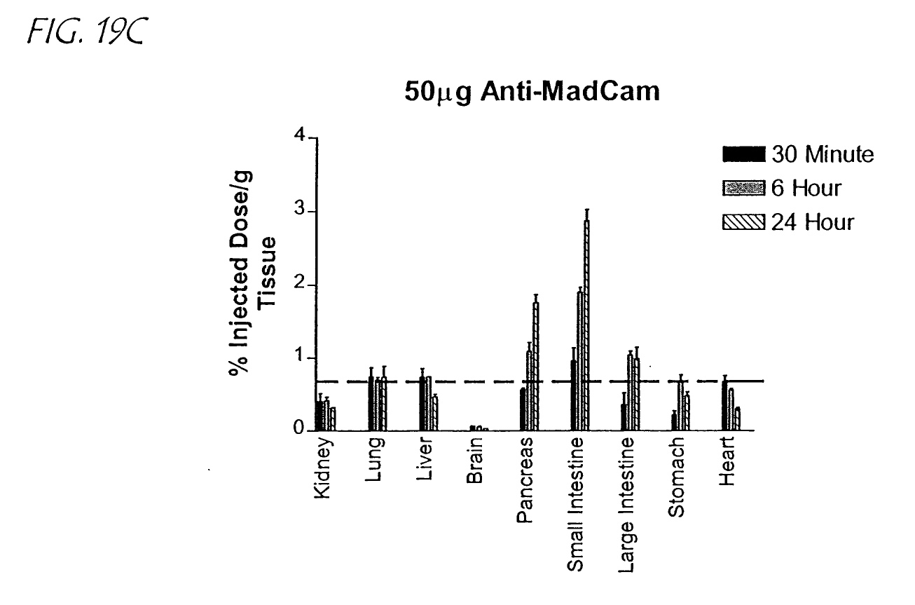

In additional experiments, the amount of OST-2 (anti-MadCam-1) antibody localized in each tissue type was determined at specific time points for each antibody dose that was administered. IgG1 isotype anti-influenza monoclonal antibodies were used as a control for background fluorescence. FIGS. 19A-C show the weight percent of OST-2 that was present in each tissue at each time point tested for each dosage level. Specifically, FIG. 19A shows that about 3% of the total 5 μg dose localized to the pancreas after 6 hours. Greater than 5% of the dose was observed in the small intestine after the same amount of time. The amount of OST-2 localized to non-GI tissues was generally less than 0.75% of the dose weight, which corresponds to the maximum levels of anti-influenza control antibody that localized to each tissue type (FIG. 19A, dashed line). It should be noted, that compared to the lungs, the pancreas is poorly vascularized. Accordingly, the percentage of antibody dose that is bound to this small area would be expected to be lower than for a antibody ligand that binds to a highly vascularized tissue such as the lung.

Results similar to those obtained for the 5 μg doses were also obtained for the 20 and 50 μg doses (FIGS. 19B and 19C, respectively). Additionally, the amounts of anti-influenza IgG1 isotype control antibody localized to each tissue was also similar to the amounts localized at the 5 μg dose level. There was at least one notable difference between the 5 μg dose and the two higher doses, however. At the 5 μg dosage, the amount of OST-2 localized in the GI organs peaked after 6 hours (FIG. 19A) and by 24 hours they began to fall. At higher doses, localization occurred in the pancreas and other GI organs cumulatively over the 24 hour time period. (FIGS. 19B-C). Taken together, these results indicate that high levels of OST-2 localize specifically to the GI organs, such as the pancreas, and the levels of this antibody increase over time. Such high levels of localization will likely result in a significant improvement in the therapeutic index of any drug delivered using this antibody ligand.

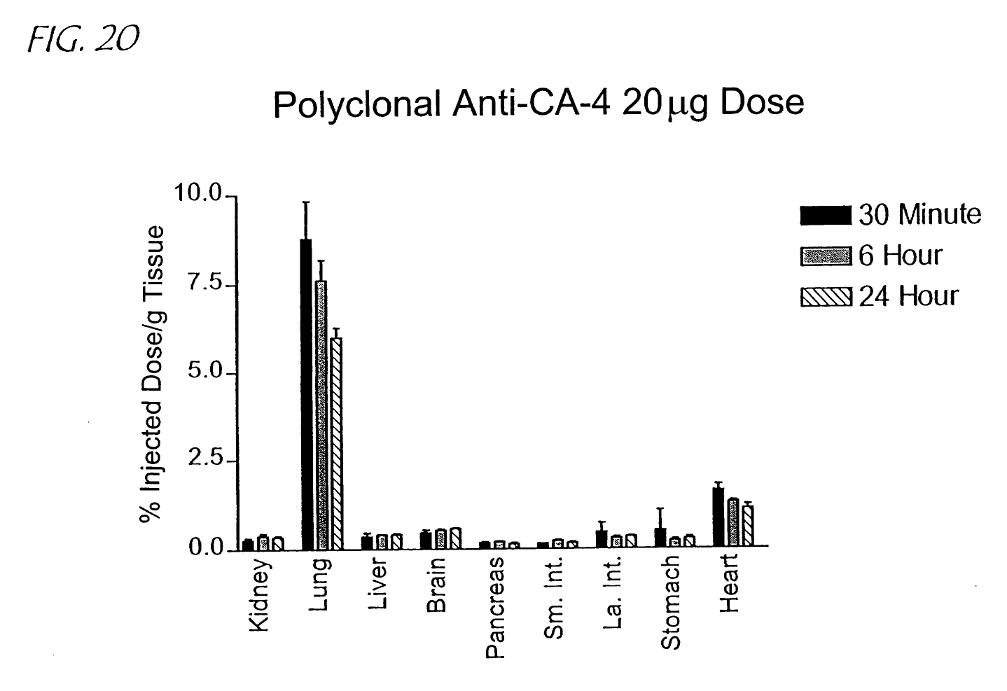

In similar experiments, 20 μg of Europium-labeled anti-CA-4 antibody ligand was administered intravenously to rats and the amount of ligand that localized in each tissue type was determined at specific time points. The affinity-purified rabbit polyclonal antibody to CA-4 (anti-CA-4), which was prepared as described in Example 8, was used as the tissue specific ligand. FIG. 20 shows that approximately 8.5% of the total injected antibody dose localized to the lung within the first 30 minutes. Approximately 2% of the antibody was found in the heart after the same time period. Levels of antibody in both the heart and lung slightly decreased after 6 hours then continued to decline when measured again at 24 hours. Anti-CA-4 did not accumulate significantly in any other tissues during the 24 hour timecourse.

### Example 12

### Quantification of Antibody Ligand Bound to Luminally Expressed Target Protein by Scintigraphy

The following example describes an alternative means for quantitatively analyzing antibody ligands localized to luminally expressed target proteins in various target tissues. OX-61 antibodies, which are specific for DPP-4, were radio-labeled with 1251 then either 1 μg or 5 μg doses were injected into the tail veins of Sprauge-Dawley rats and allowed to circulate for 5 minutes, 2 hours or 8 hours. Numerous tissues and fluids were analyzed by scintigraphic methods that are well known in the art. Results of the scintigraphy were expressed as nanogram equivalents of antibody per gram of tissue in each organ. The percentage of injected dose that localized to a particular organ was calculated using the known average weight of rat organs.

Using the above method, OX-61 was found to localize predominately to the lung. At both doses, OX-61 localized to the lung within the first five minutes. After two hours, 22% of the total injected 1 μg dose was found localized in this tissue. After 8 hours, the amount of antibody found in the lung increased to 30% of the injected dose. OX-61 was also found in the liver. Initially, a high level of OX-61 was observed in the liver; however, after 8 hours only 7% of the injected dose remained. Initial detection in the liver followed by the rapid decrease was most likely due to antibody circulating in the blood.

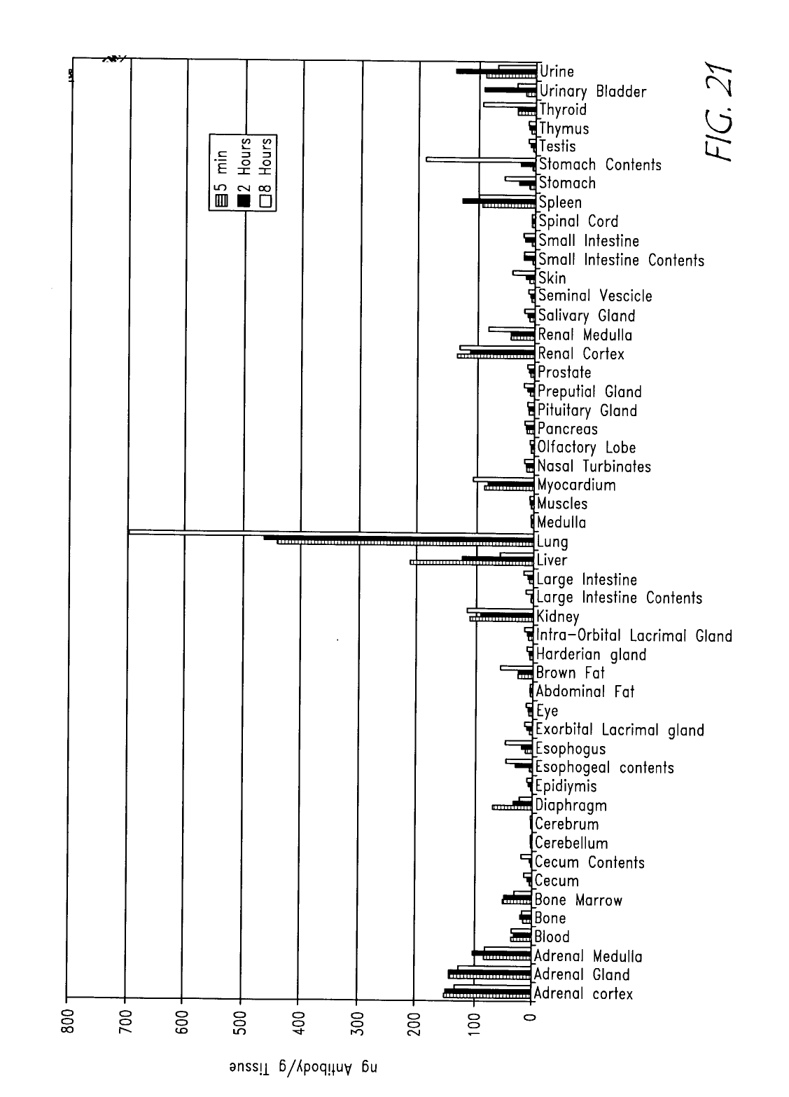

The results were similar when a 5 μg dose was administered. FIG. 21 shows that more than 0.4 μg of OX-61 per gram of tissue (20% of the initial antibody dose) localized to the lung after the first five minutes. After 8 hours, the amount of OX-61 increased to approximately 0.7 μg of OX-61 per gram of lung tissue. Throughout the timecourse, there was no significant build-up of OX-61 in any other tissue. These results confirm that high levels of OX-61 localize specifically to the lung and the levels of antibody remain high over a long period of time.

### Example 13

### Transcytosis of Antibody Ligands by Luminally Expressed Target Proteins

The following example describes methods that were used to characterize transcytotic, luminally expressed target proteins in terms of their ability to mediate transcytosis. More specifically, three-color histology was used to characterize luminally expressed target proteins capable of transporting bound ligand from the luminal surface of the blood vessel to the surrounding tissue space. Of the target proteins examined, only DPP-4 and CD90 appeared to have the ability to mediate transcytosis across the endothelial cell layer.

Three-color histology was performed using specific antibody ligands and stains specific for cellular structures. As in previous examples, antibodies specific to DPP-4, MadCam-1, CD90 or CA-4 were injected into the tail veins of Sprauge-Dawley rats in 50 μg doses. After 30 minutes, the rats were sacrificed and their organs were prepared for histology as previously described in Example 8. The tissue sections were then incubated with Cy3-labeled secondary antibodies in order to detect bound primary antibodies. Additionally, the tissue sections were stained with 4′, 6-diamidino-2-phenylindole, dihydrochloride (DAPI) and fluorescein-labeled Griffonia simplicifolia Lectin 1-isolectin B4 (GSL-1). DAPI stains the nuclei of the cells blue and GSL-1 stains the endothelium green. Transcytosis of antibody across the endothelium was detected by determining the distribution of yellow regions which were produced by the mixing of the red Cy-3 signal with the green-stained endothelium as antibody was transported across this cell layer.

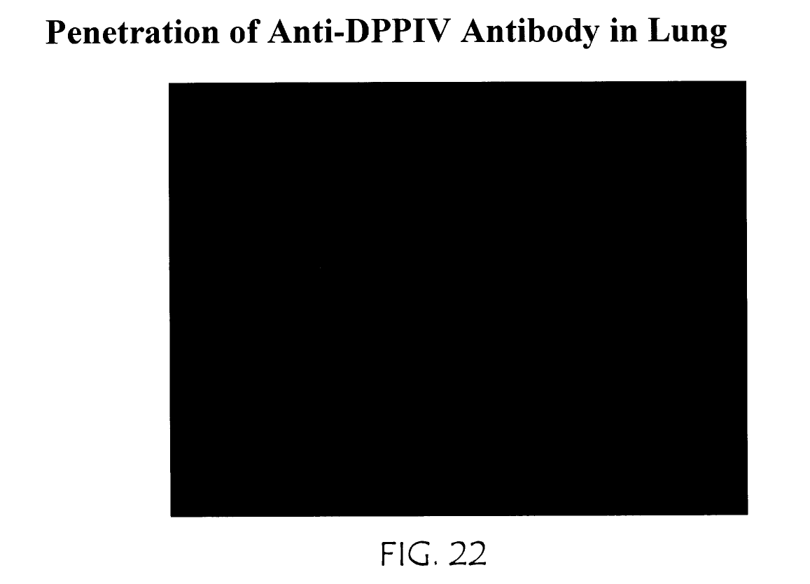

Using the above-described method, the transcytotic transport of OX-61 by DPP-4 was detected. FIG. 22 shows that OX-61 penetrated into the lung tissue surrounding the vasculature. As expected the surfaces of capillaries were stained green and cell nuclei were stained blue. Air-spaces in the lung were represented as black areas. The presence of yellow distributed throughout the endothelium indicated that the antibody was transported across the endothelial barrier and into the interstitial lung tissue.

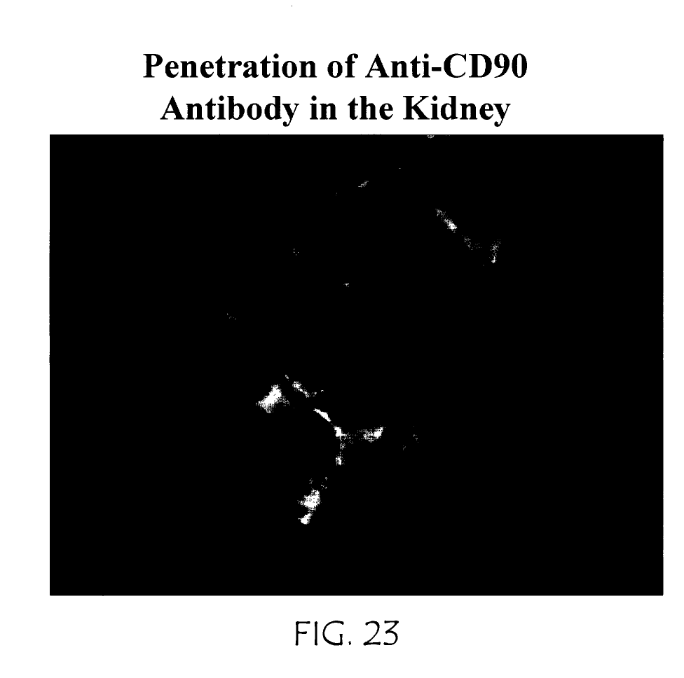

Similarly, the transcytotic transport of OX-7 by CD90 was detected. FIG. 23 shows that OX-7 penetrated into the glomerulus of the kidney. The penetration was indicated by the substantial amount of mixing that was observed between the bound antibody and the endothelium. This distribution of antibody into the endothelium can be seen in FIG. 23 as a diffuse area of yellow located between the red staining antibody that is bound at the luminal surface and the green staining endothelial layer.

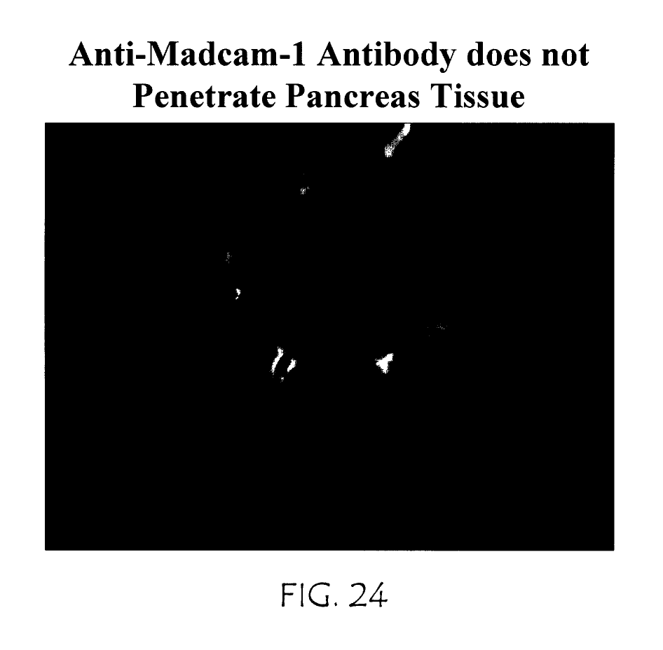

Although OST-2 bound to MadCam-1 as expected, the antibody was not transported across the endothelium into the pancreas. FIG. 24 shows a section of the pancreas having no visible penetration of antibody into the endothelium. The antibody localized to the surface of the blood vessel (red) but never moved across the endothelium (green) and into the surrounding tissue. The absence of any yellow coloring in FIG. 24 demonstrates this lack of transcytosis.

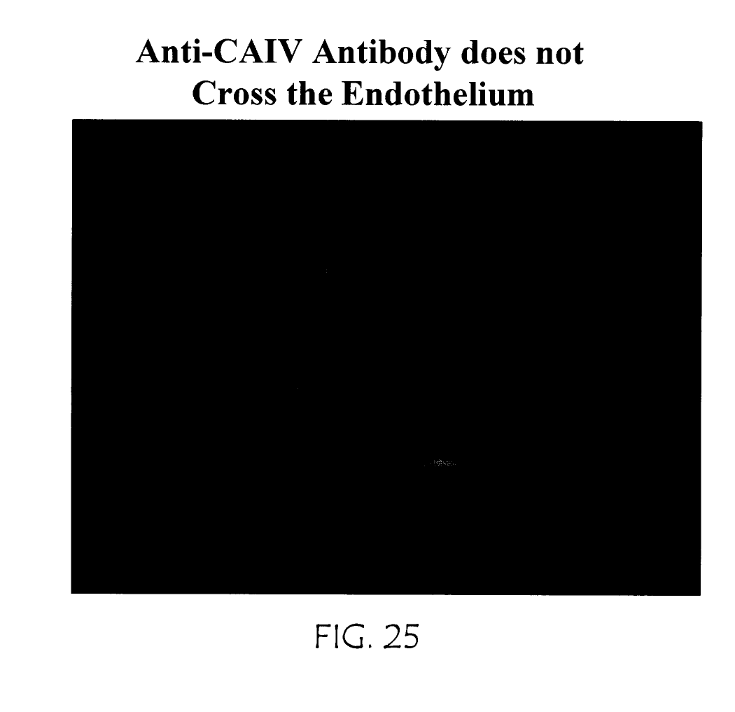

Similarly, no transcytosis was seen for anti-CA-4 antibody that was bound to CA-4 on the luminal surface of the vasculature of the lung. FIG. 25 shows a section of the lung having no visible penetration of antibody into the endothelium. In other words, the red areas of antibody bound to the endothelial surface never moved into the endothelial layer. This lack of movement is noted in FIG. 25 by the absence of yellow color intermixed in the endothelial cell layer. Similar results were noted for anti-CA-4 antibody that localized to the heart.

Taken together, the above results indicate that the luminally expressed target proteins that are identified herein are useful for both the delivery of drugs to the interstitium of specific tissues as well as their vascular surfaces.

Examples 14-16 describe therapeutic complexes comprising target-protein-specific antibody ligands that are linked to therapeutic moieties such as gentamicin and doxorubicin.

### Example 14

### Selective Drug Delivery to Tissues Using Specific Target Proteins

The following example describes the delivery of therapeutic complexes to specific target tissues. Therapeutic complexes were constructed by coupling mouse monoclonal antibodies specific to DPP-4 or MadCam-1 to either gentamicin or doxorubicin via a non-cleavable linker using methods well known in the art. On average, three molecules of drug were covalently conjugated to each antibody. Approximately, 50 μg of each therapeutic complex was administered to rats by tail vein injection and allowed to circulate for 30 minutes. The rats were then sacrificed and their organs were sectioned for histology using the method described in Example 8. Gentamicin and doxorubicin therapeutic complexes were detected by addition of either gentamicin- or doxorubicin-specific antibodies as appropriate, followed by signal amplification with Cy3 conjugated secondary antibodies. In some experiments, the tissue sections were also stained with 4′, 6-diamidino-2-phenylindole, dihydrochloride (DAPI) and fluorescein-labeled Griffonia simplicifolia Lectin 1-isolectin B4 (GSL-1) to demonstrate transcytosis (Three-color histology methods as described in Example 13).

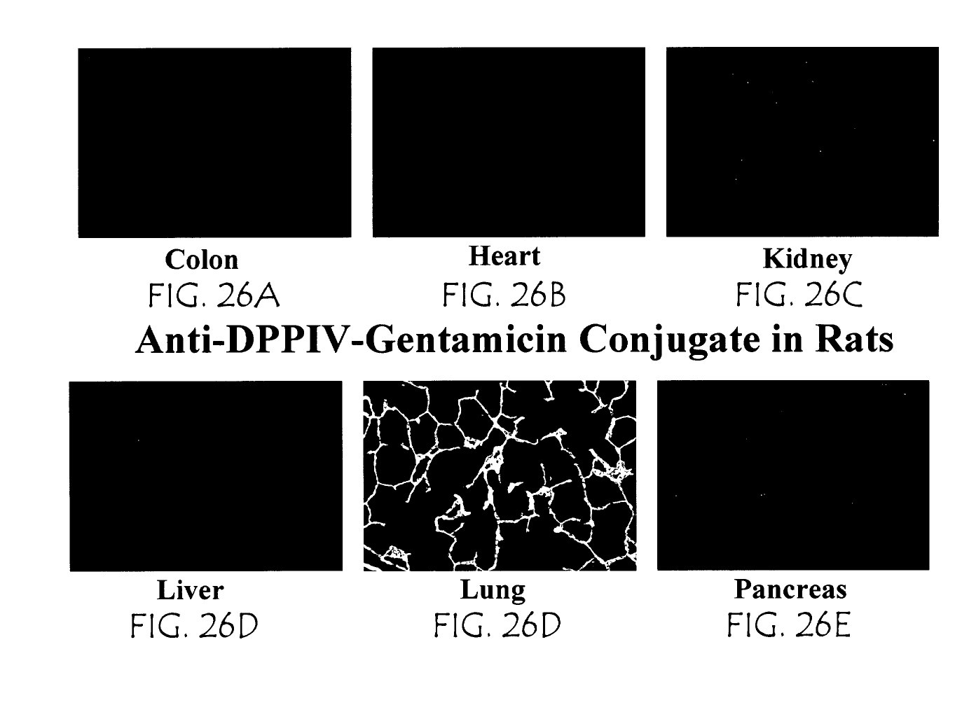

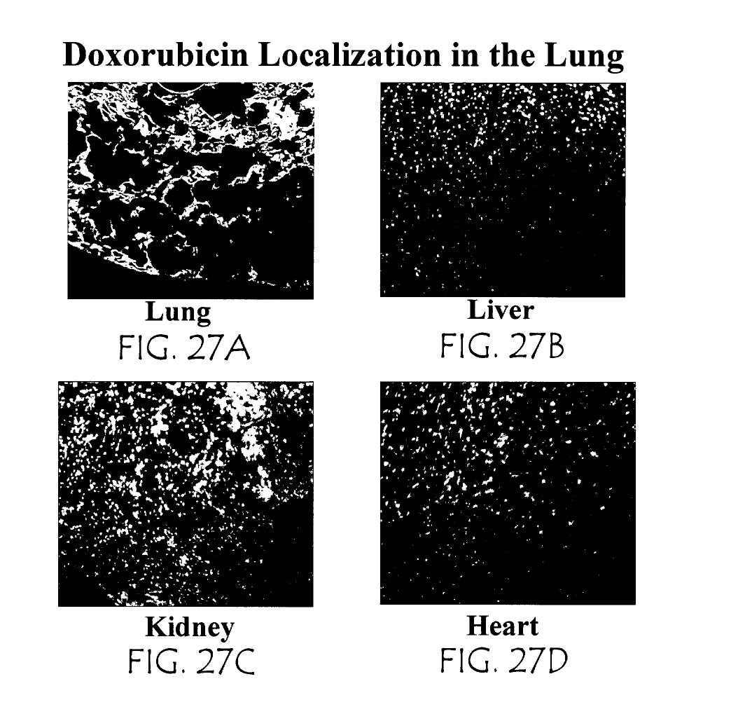

Using the above-described methods, OX-61/gentamicin and OX-61/doxorubicin therapeutic complexes were found to localize specifically to the lung tissue within 30 minutes after the initial injection. FIGS. 26A-F shows the binding of the OX-61/gentamicin therapeutic complex to specific tissues. Specifically, this therapeutic complex was observed in lung within thirty minutes following its injection (FIG. 26E). It was not present, however, in any other of the tissues examined (FIGS. 26A-D and 26F). Similar results were obtained for the OX-61/doxorubicin therapeutic complex (FIGS. 27A-D).

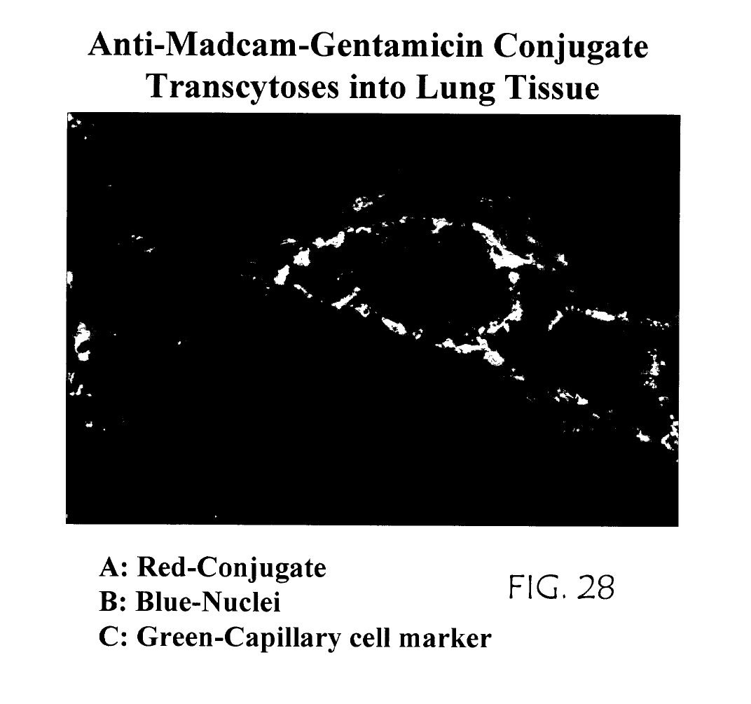

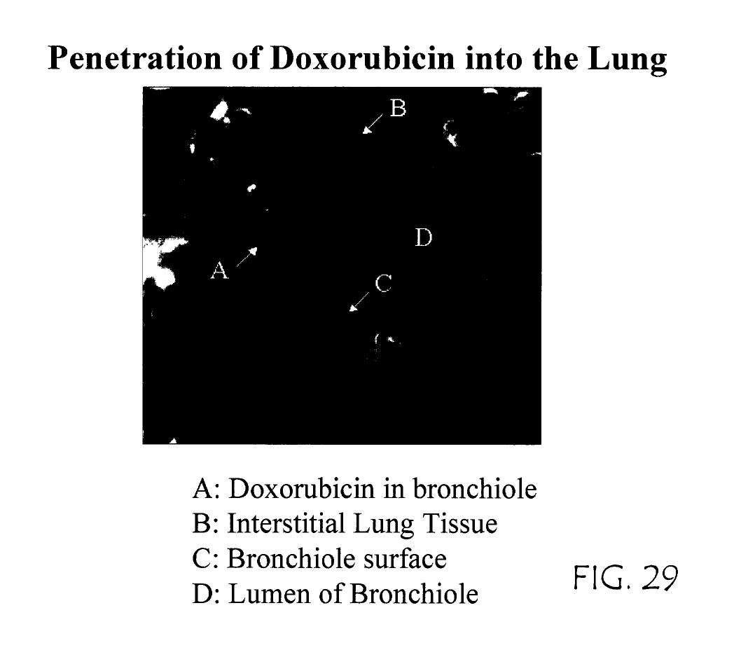

Using the above-described three color histology methods, DPP-4-mediated transcytotic transport of both OX-61/gentamicin and OX-61/doxorubicin therapeutic complexes was detected. FIG. 28 shows that the OX-61/gentamicin therapeutic complex penetrated the endothelium then localized into the interstitium of the lung. Therapeutic complexes were observed lining the capillaries and throughout the endothelial cell layer. Complexes were also observed throughout the interstitial tissues of the lung. The areas of yellow in FIG. 28 show the movement of the therapeutic complex across the endothelium. Similar results were seen for the OX-61/doxorubicin therapeutic complex. FIG. 29 specifically shows the accumulation of this therapeutic complex in the interstitium of the lung (FIG. 29, arrow B).

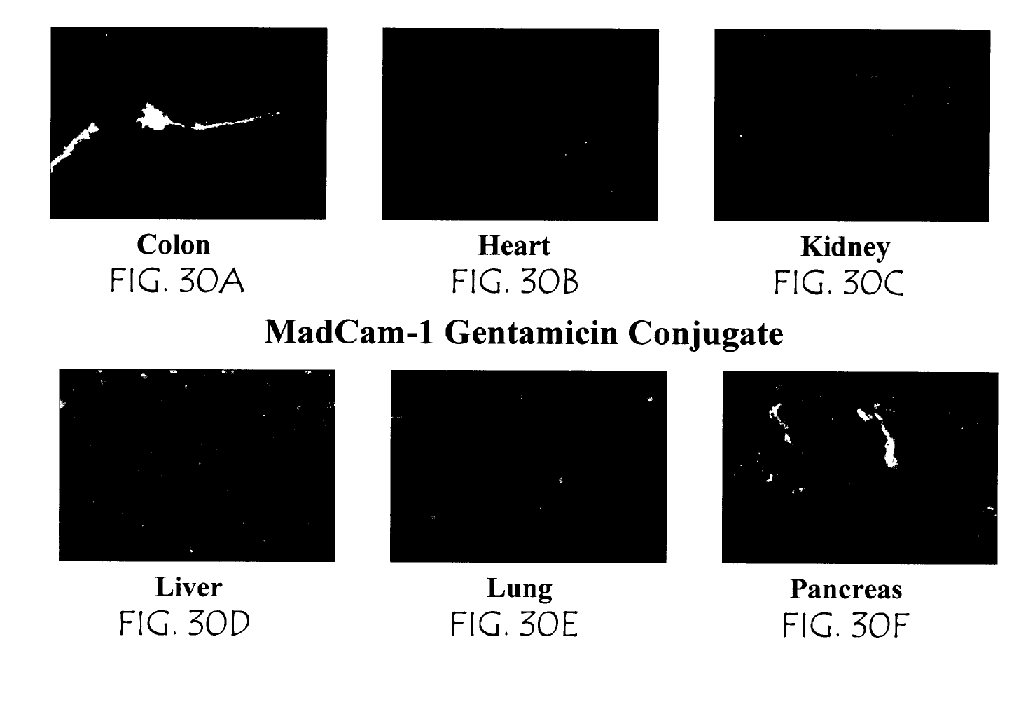

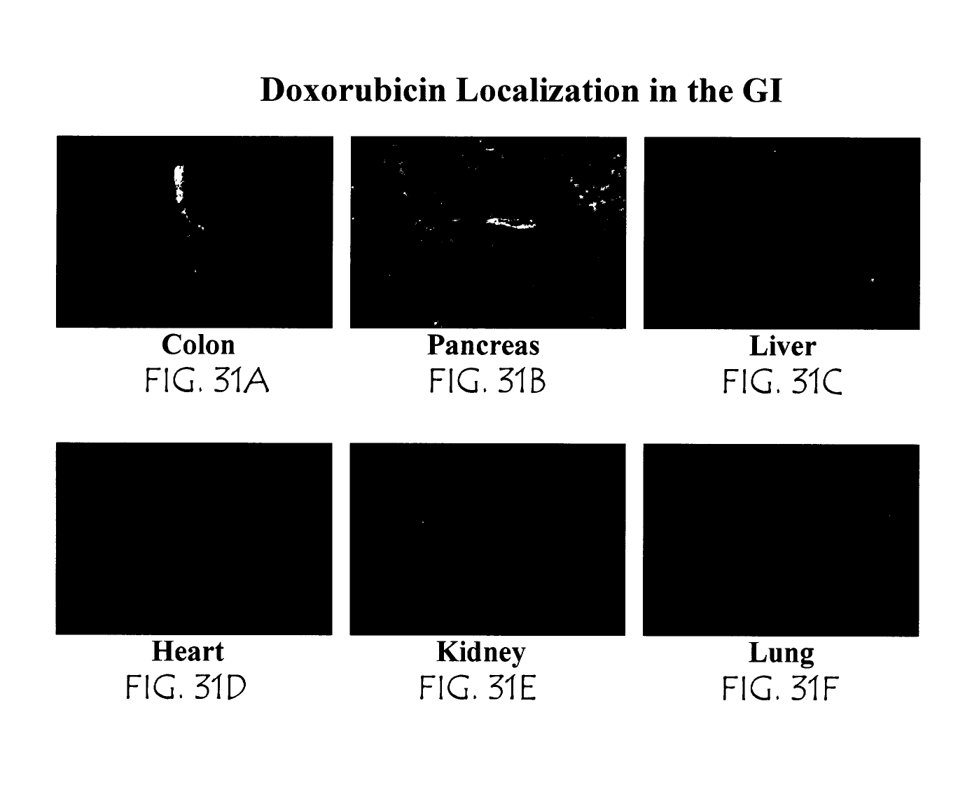

The tissue specific localization of OST-2/genatmicin and OST-2/doxorubicin conjugates was also evaluated. FIGS. 30A and 30F show that the OST-2/gentamicin conjugate specifically bound to MadCam-1 in both the colon and the pancreas. This conjugate did not localize to any of the other tissues that were tested (FIG. 30B-E). Similar results were observed for the OST-2/doxorubicin therapeutic complex (FIG. 31 A-F).

### Example 15

### Targeted Liposomal Formulations of Gentamicin Using the DPP-4-Specific Antibody OX-61

The following example describes the delivery of liposomal therapeutic complexes to specific target tissues. Therapeutic complexes were constructed by coupling mouse monoclonal antibodies specific to DPP-4 (ligand) to gentamicin (therapeutic moiety) using liposomes (linker). The liposomes were constructed using either egg phosphatidylcholine (EPC) or disteroylphosphatidylcholine (DSPC) as the main phospholipid component (greater than 50 mole percent). Maleimido-pegylated disteroylphosphatidylethanolamine (MPDSPE) was added as a minor lipid component in a concentration of about 5 mole percent. MPDSPE was synthesized by coupling polyethylene glycol (PEG) having a molecular weight of about 5000 kDa to disteroylphosphatidylethanolamine (DSPE). The free end of the attached PEG group was then converted to a reactive maleimide using methods well known in the art. The liposome formulation was completed by adding cholesterol in a concentration ranging from 0 to 50 mole percent depending on the amount of phophospholipid that was initially used.

Therapeutic complexes were generated by coupling both gentamicin and OX-61 to the liposome linkers. Gentamicin sulfate was coupled by passively entrapping it within the liposomes during their formation. Gentamicin was entrapped at a concentration of approximately 150 μg/ml. Following the entrappment of the therapeutic moiety, the OX-61 antibody was coupled to the liposome linker. This coupling was accomplished by first reacting OX-61 with Traut's reagent to convert primary amines to thiols. The antibody was then coupled to the reactive MPDSPE.

The biodistribution of gentamicin administered in EPC and DSPC liposomes targeted to DPP-4 (EPC-DPP and DSPC-DPP therapeutic complexes, respectively) was compared to that of free gentamicin and gentamicin that was administered in untargeted liposomes. Specifically, a solution of free gentamicin or a dispersion containing therapeutic complexes or liposomes having no ligand bound to their surface was injected into the tail veins of Sprauge-Dawley rats at a dose of 150 μg gentamicin per rat. The rats were sacrificed after either 30 minutes or 18 hours and their organs were removed and homogenized. The amount of gentamicin in each organ homogenate was measured using a TDX analyzer (Abbott). At least three independent experiments were performed for each gentamicin formulation at each time point.

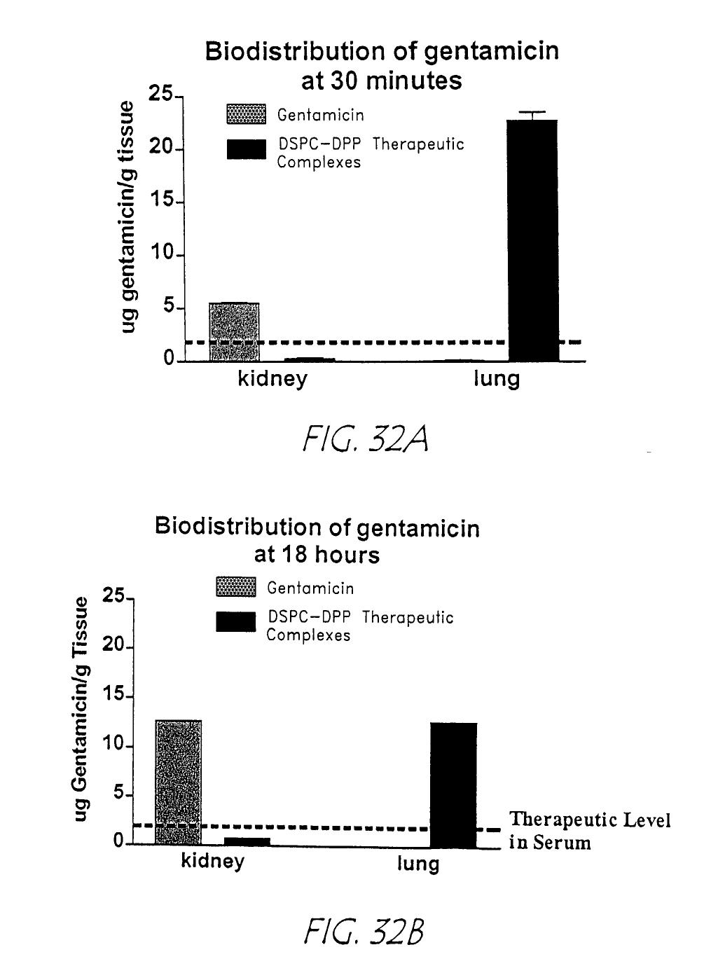

Using the above methods, the amount of gentamicin that localized to the lungs and kidneys after administration was determined for both free gentamicin and gentamicin administered in DSPC-DPP therapeutic complexes. In particular, within 30 minutes after administration, free gentamicin began to accumulate in the kidney (FIG. 32A). After 18 hours, the amount of gentamicin present in the kidneys more than doubled (FIG. 32B). In contrast, even after 18 hours, very little gentamicin appeared in the kidneys when administered in DSPC-DPP therapeutic complexes (FIGS. 32A-B). Nearly opposite effects were seen in lung tissue. FIGS. 32A-B show that, when administered in its free form, very little gentamicin was observed in the lungs either 30 minutes or 18 hours after injection. However, when administered in a DSPC-DPP therapeutic complex, gentamicin was present at about 20 μg per gram of lung tissue after 30 minutes (FIG. 32A). After 18 hours, the level fell by about half (FIG. 32B). These results indicated that build up of gentamicin in the kidneys, and thus gentamicin- mediated toxicity, can be prevented by delivering this drug specifically to the site of infection using appropriately targeted liposomal therapeutic complexes.

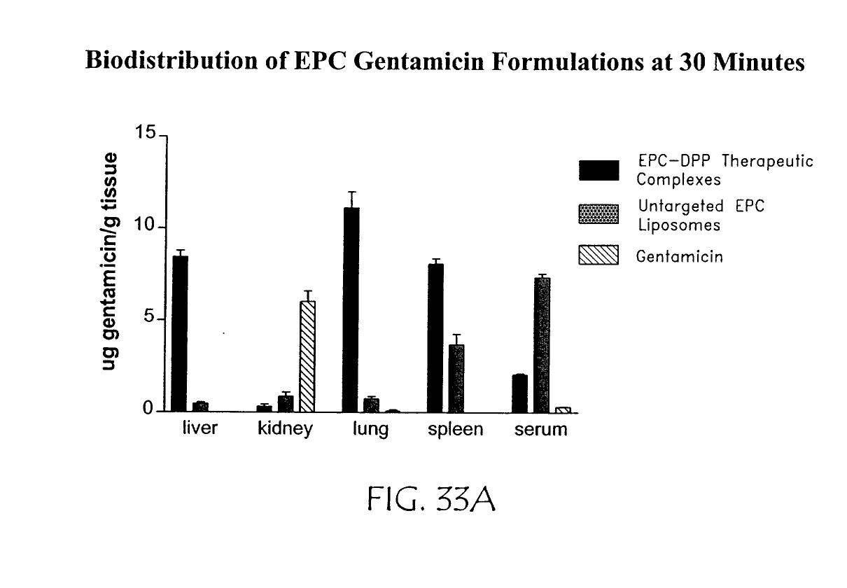

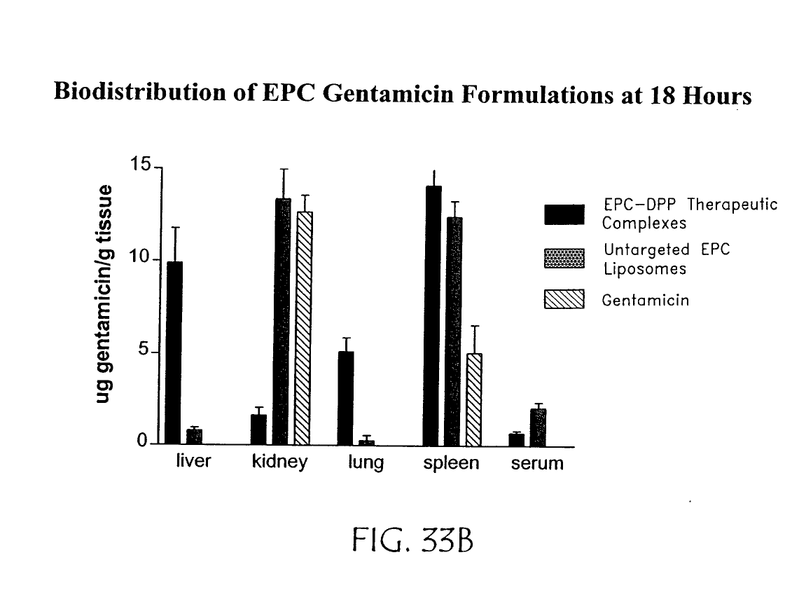

The biodistribution of free gentamicin was compared with that of gentamicin delivered in EPC-DPP therapeutic complexes and untargeted EPC liposomes. Within 30 minutes after administration of free gentamicin, a substantial amount of this compound appeared in the kidneys. After 18 hours, this amount more than doubled (FIGS. 33A-B). Gentamicin delivered in untargeted liposomes, appeared predominately in the serum after 30 minutes, but substantial amounts were detected in both the kidney and the spleen after 18 hours (FIGS. 33A-B). In contrast, within 30 minutes, most of the gentamicin delivered in EPC-DPP therapeutic complexes was distributed between the lung, liver and spleen but very little was observed in the kidneys or serum. The highest level of gentamicin, about 15% of the injected dose, was detected in the lung (FIG. 33A). Similar distributions were observed after 18 hours (FIG. 33B).

The above results indicate that gentamicin was targeted to lungs using EPC-DPP therapeutic complexes. Although the amount of gentamicin appearing in the liver and the spleen was significant, it is likely that the amount of drug accumulating in these organs can be reduced. Such a result can be achieved by using antibody fragments rather than whole antibodies as the targeting ligand. It has been well established that the Fc portion of antibodies mediate uptake into the liver and spleen. Accordingly, removing this portion of the antibody would likely reduce accumulation in these organs. Although accumulation of gentamicin in the kidney could not be prevented using untargeted liposomes, gentamicin could be effectively shielded from the kidney using the EPC-DPP therapeutic complex. Accordingly, such complexes are useful for both targeted drug delivery and preventing drug toxicity.

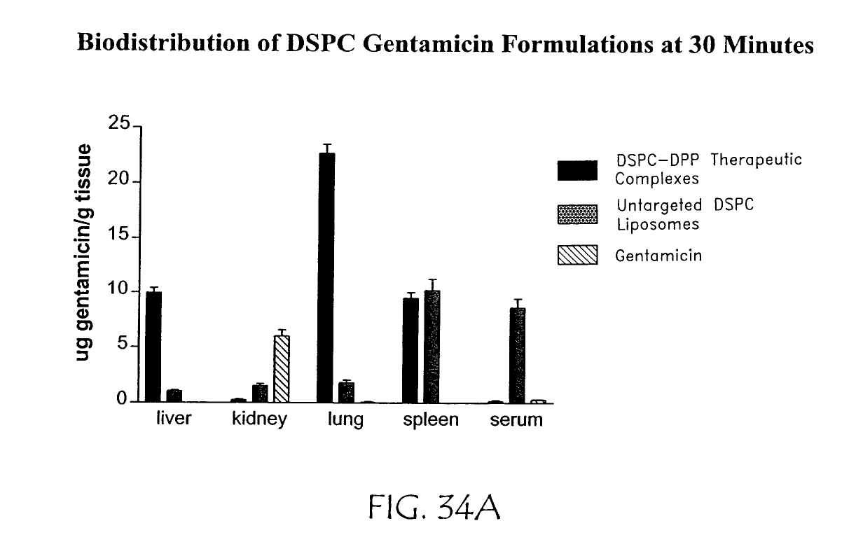

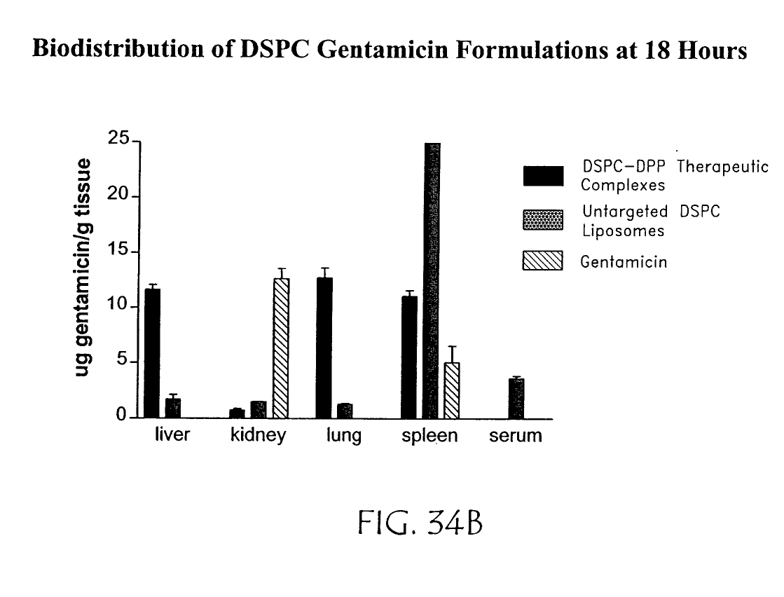

The biodistribution of free gentamicin was also compared with that of gentamicin delivered in DSPC-DPP therapeutic complexes and untargeted DSPC liposomes. FIGS. 34A-B show that the biodistribution of gentamicin delivered in DSPC-DDP therapeutic complexes both after 30 minutes and 18 hours was similar to that of gentamicin delivered in EPC-DPP therapeutic complexes with one significant difference. At both time points, DSPC-DPP therapeutic complexes localized over twice the amount of gentamicin in the lungs as EPC-DPP therapeutic complexes. (FIGS. 34A-B and 33A-B). The biodistribution of gentamicin delivered in untargeted DSPC liposomes was also similar to that of gentamicin delivered in untargeted EPC liposomes except far less gentamicin was found in the kidney after 18 hours when using DSPC liposomes for delivery (FIGS. 34A-B and 33A-B).

Taken together the above results indicate that DSPC-DPP therapeutic complexes were capable of targeting high levels of gentamicin to the lung. In addition, the use of such therapeutic complexes prevents the build up of gentamicin in the kidneys where it is known to have toxic effects.

### Example 16

### Efficacy of Therapeutic Complexes Containing Gentamicin

The following example describes the efficacy of EPC-DPP therapeutic complexes containing gentamicin in the treatment of pneumonia. Pneumonia was established in fifteen rats by infecting each animal with 1.5×10⁷ Klebsiella pneumoniae via intratracheal injection. The rats were then divided into three groups having five animals each. After 24 hours, one group was treated by administering 5 mg/kg of free gentamicin per animal. A second group was treated by administering 5 mg/kg of gentamicin formulated in EPC-DPP therapeutic complexes per animal. The final group was left untreated as a control group. The rats were then monitored for survival over the next fifteen days.

The gentamicin delivered in EPC-DPP therapeutic complexes was superior to free gentamicin for the treatment of pneumonia. Only one of the five animals died in the EPC-DPP-treated group. This death occurred on day six. Each of the other four animals survived through day fifteen and displayed no signs of infection. Additionally, one of the surviving animals was sacrificed and no pathogenic bacteria were found in the lung. These results indicated that the gentamicin delivered in the EPC-DPP therapeutic complexes had completely cured the infection in 80% of the rats treated.

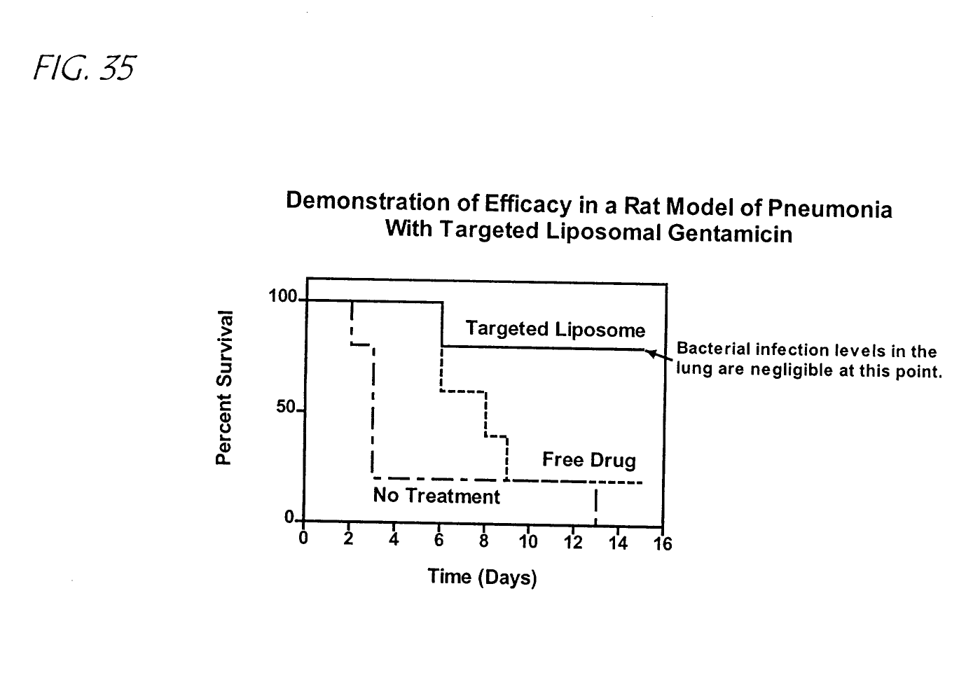

In contrast, all of the untreated rats died. Four of these animals died by day three. Four of the five animals treated with free gentamicin died by day nine. However, one animal did survive to day 15. Accordingly, the efficacy of free gentamicin was much less than that of gentamicin delivered to the lung in EPC-DPP therapeutic complexes (FIG. 35).

In Examples 17-22, the lung-specific luminally expressed molecule rat dipeptidyl peptidase IV (DPP-4) is used to produce a number of therapeutic complexes which are used to treat a variety of lung-specific diseases or deficiencies.

### Example 17

### Use of DPP-4 Doxorubicin Therapeutic Complex with an Acid Sensitive Linker for the Treatment of Lung Cancer

Initially, a therapeutic level of a human doxorubicin/DPP-4 complex such as that from Example 7 is administered to a patient intravenously. An effective amount of the complex is delivered to the patient, preferably 1 μg to 100 mg/Kg of patient weight in saline or an intravenously acceptable delivery vehicle. The DPP-4 F(ab′)₂ is specific for the lung tissue. As the therapeutic complex is transcytosed into the lung tissue, the acid sensitive linker is cleaved and the doxorubicin is free to intercalate into the DNA. Because the doxorubicin is incorporated into the DNA of cycling cells, the effect on the cancer cells which are in the process of cycling will be marked and the effect on the normal lung cancer cells much reduced. Therefore, the treatment results in a reduction of the number of cancer cells in the lung, with a minimum of side effects. Because doxorubicin generally targets dividing cells and, because of the tissue specificity, it will only affect the dividing cells of the lung, and therefore, it is envisioned that the number of cells killed due to side effects of the treatment will be minimal.

In Example 18 a method is set out for the synthesis and use of a DPP-4/doxocillin prodrug treatment for lung cancer.

### Example 18

### Use of DPP-4/doxocillin Therapeutic Complex for the Treatment of Lung Cancer Using a Prodrug

The therapeutic complex is a DPP-4/β-lactamase conjugate which includes an F(ab′)₂ specific for DPP-4 linked to β-lactamase via a polypeptide linker, or a covalent bond. The linker used was SMCC. The chemotherapeutic agent doxocillin does not cross the endothelium due to a number of negative charges in the structure, which makes it nontoxic for all cells and ineffective as an anticancer drug. However, doxocillin can be thought of as a pro-drug which becomes active upon cleavage of the β-lactam ring to produce doxorubicin. Doxorubicin does cross the endothelium and intercalates into the DNA of cycling cells, making it an effective chemotherapeutic agent.

Initially, a therapeutic amount of a DPP-4/β-lactamase complex is administered to the patient intravenously. The DPP-4 F(ab′)₂ is linked to the β-lactamase prodrug in the therapeutic complex using a linker which is not cleavable. The DPP-4 F(ab′)₂ ligand is targeted to the lung tissue. A therapeutic level of the therapeutic complex is administered to the patient at between about 1 μg to 100 mg/Kg of patient weight. After administration and localization of the therapeutic complex, a therapeutic level of doxocillin is administered to the patient at between about 1 μg to 100 mg/Kg of patient weight, preferably between 10 μg to 10 mg/Kg of patient weight. The doxocillin is taken up systemically, but only in the microenvironment of the lung, the doxocillin is cleaved by the β-lactamase to produce doxorubicin. Therefore, the eukaryotic cytotoxic activity of the prodrug is unmasked only at the location of the β-lactamase, that is, the lungs. The doxorubicin is taken up by the lung tissue and intercalates into the DNA. However, because the doxorubicin is incorporated into the DNA of cycling cells, the effect on the cancer cells which are in the process of cycling will be marked and the effect on the normal lung cancer cells much reduced. The treatment results in a reduction in the number of cancer cells in the lung.

In Example 19 a method is set out for the synthesis and use of a DPP-4/cephalexin prodrug therapeutic complex to treat pneumonia.

### Example 19

### Use of DPP-4 Therapeutic Complex for the Treatment of Lung Infections

The most common bacterial pneumonia is pneumococcal pneumonia caused by Streptococcus pneumoniae. Other bacterial pneumonias may be caused by Haemophilus influenzae, and various strains of mycoplasma. Pneumococcal pneumonia is generally treated with penicillin. However, penicillin-resistant strains are becoming more common.

The present invention is used for the treatment of pneumococcal pneumonia in humans (or other mammals) as follows: A therapeutic complex is constructed by linking the F(ab′)₂ fragment of human DPP-4 antibodies to cephalexin. The linker used is a liposome. The liposomes are constructed so that the F(ab′)₂ fragment is incorporated into the membrane and the cephalexin is carried within the liposome. Liposomes are produced by polymerizing the liposome in the presence of the DPP-4/F(ab′)₂ ligand such that the ligand becomes a part of the phospholipid bilayer and are prepared using the thin film hydration technique followed by a few freeze-thaw cycles. However, liposomal suspensions can also be prepared according to method known to those skilled in the art. 0.1 to 10 nmol of the therapeutic complex is injected intravenously. The liposomes carrying the cephalexin are targeted to the lung by the DPP-4 specific F(ab′)₂ fragments. Upon binding to the endothelium, the liposomes are taken up and the cephalexin is taken into the lung tissue. The cephalexin can then act on the cell walls of the dividing S. pneumonia organisms. One advantage of the targeting of antibiotics to a specific region is that less antibiotic is needed for the same result, there is less likelihood of side effects, and the likelihood of contributing to the drug resistance of the microorganism is considerably reduced.

In Example 20 a method is set out for the synthesis and use of a DPP-4/rifampin prodrug therapeutic complex to treat tuberculosis.

### Example 20

### Use of DPP-4 Therapeutic Complex for the Treatment of Tuberculosis

It can readily be envisioned that diseases such as tuberculosis, caused by the bacterium M. tuberculosis, which is often treated using rifampin or isoniazid for a very long period of time, would be more effectively treated using the therapeutic agent of the present invention. Much of the reason for the high incidence of disease and drug resistance in this microbe is the noncompliance with the extremely long course of treatment. It can be envisioned that using a method that directly targets the lungs with a high concentration of antibiotic would reduce the need for an unworkably long treatment and thus reduce the incidence of noncompliance and drug resistance.

The preferred embodiment is used for the treatment of tuberculosis in humans (or other mammals) as follows: A therapeutic complex is constructed by linking the F(ab′)₂ fragment of human DPP-4 antibodies to rifampin. The linker used is a liposome. The liposomes are constructed so that the F(ab′)₂ fragment is incorporated into the membrane and the rifampin is carried within the liposome. Liposomes are produced by polymerizing the liposome in the presence of the DPP-4/F(ab′)₂ ligand such that the ligand becomes a part of the phospholipid bilayer and are prepared using the thin film hydration technique followed by a few freeze-thaw cycles. However, liposomal suspensions can also be prepared according to method known to those skilled in the art. 0.1 to 10 nmol of the therapeutic complex is injected intravenously. The liposomes carrying the rifampin are targeted to the lung by the DPP-4 specific F(ab′)₂ fragments. Upon binding to the endothelium, the liposomes are taken up and the rifampin is taken into the lung tissue. The rifampin can then act on the M. tuberculosis organisms.

In Example 21, a method is set out for the synthesis and use of a DPP-4/surfactant protein therapeutic complex to treat lung diseases resulting from under-production of surfactant proteins.

### Example 21

### Use of DPP-4 Therapeutic Complex for the Treatment of Surfactant Deficiencies

A number of lung diseases, including emphysema, include, as part of the cause or effect of the disease, deficiencies of surfactant proteins. The present invention is used for the treatment of surfactant deficiencies as follows: A therapeutic complex is constructed by linking the F(ab′)₂ fragment of DPP-4 antibodies to a surfactant protein such as SP-A (surfactant protein A). The linker used is a pH sensitive bond. The therapeutic complex is injected intravenously into a patient's veins and is targeted to the lung by the DPP-4 specific F(ab′)₂ fragments. Upon binding to the endothelium, the therapeutic complex is transcytosed by the lung tissue and the change in pH cleaves the bond, thus releasing the surfactant protein.

In Example 22, a method is set out for the synthesis and use of a DPP-4/corticosteroid therapeutic complex to treat rejection of transplanted lung tissue.

### Example 22

### Use of DPP-4 Therapeutic Complex for the Treatment of Lung Transplantation Rejection

The present invention is used for the treatment of lung transplantation rejection as follows: a therapeutic complex is constructed by linking the F(ab′)₂ fragment of DPP-4 antibodies to an immunosuppressant such as a corticosteroid or cyclosporin with a pH sensitive linker. The therapeutic complex is injected intravenously into a patient's veins and is targeted to the lung by the DPP-4 specific F(ab′)₂ fragments. Upon binding to the endothelium, the therapeutic complex is transcytosed or taken up by the lung tissue and the change in pH cleaves the bond, thus releasing the immunosuppressant only in the area of the lungs. It can readily be seen that the advantage of such a treatment is that the patient is not immunosuppressed and still has a healthy active immune system during recovery from the surgery. The lung (or other transplanted organ) is the only organ which is immunosuppressed and is carefully monitored.

One skilled in the art will appreciate that these methods and compositions are and may be adapted to carry out the objects and obtain the ends and advantages mentioned, as well as those inherent therein. The methods, procedures, and compositions described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

Those skilled in the art recognize that the aspects and embodiments of the invention set forth herein may be practiced separate from each other or in conjunction with each other. Therefore, combinations of separate embodiments are within the scope of the invention as disclosed herein.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

Other embodiments of the invention can be envisioned within the scope of the following claims.

What is claimed is:

1. A method for delivering a therapeutic agent to a specific tissue, comprising: administering a therapeutically effective amount of a therapeutic complex, said therapeutic complex comprising: a ligand which binds to a tissue-specific luminally expressed protein, a therapeutic moiety, and a linker which links said therapeutic moiety to said ligand.

2. The method of claim 1 wherein said ligand is selected from the group consisting of proteins, peptides, and small molecules.

3. The method of claim 2, wherein said proteins are selected from the group consisting of antibodies, antibody complexes, antibody fragments, and enzymes.

4. The method of claim 1, wherein said therapeutic moiety is selected from the group consisting of enzymes, antibiotics, immunomodulators, chemotherapeutic agents, antiviral agents, antifungal agents, contrast agents, prodrugs and hormones.

5. The method of claim 4, wherein said enzymes specifically cleave prodrugs to produce the corresponding pharmaceutical agent.

6. The method of claim 1, wherein said linker is selected from the group consisting of a bond, a peptide, a liposome, and a microcapsule.

7. The method of claim 6, wherein said bond is sensitive to acidic or reducing conditions.

8. The method of claim 1 wherein an enzyme is administered between about 20 minutes and about 12 hours after administration of the therapeutic complex.

9. The method of claim 1 wherein a prodrug is administered within about 48 hours after administration of the therapeutic complex.

10. A lung and/or heart-specific therapeutic complex which interacts with a targeted endothelial cell, comprising:
a ligand which attaches said therapeutic complex to the luminal surface of a vascular endothelial cell membrane of the specific tissue, wherein said ligand binds to SEQ ID NO:9 or 11, or a homolog thereof;
a linker; and
a therapeutic moiety, wherein said linker links the ligand to the therapeutic moiety.

11. The lung and/or heart-specific therapeutic complex of claim 10, wherein said ligand is an antibody or a binding part thereof.

12. The lung and/or heart-specific therapeutic complex of claim 10, wherein said ligand does not activate a receptor.

13. The lung and/or heart-specific therapeutic complex of claim 10, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

14. The lung and/or heart-specific therapeutic complex of claim 10, wherein said linker is pH sensitive.

15. The lung and/or heart-specific therapeutic complex of claim 14, wherein said pH sensitive linker is an acid sensitive bond between the ligand and the therapeutic moiety.

16. The lung and/or heart-specific therapeutic complex of claim 10, wherein said linker is a liposome.

17. The lung and/or heart-specific therapeutic complex of claim 16, wherein said ligand is on the outside of the liposome and said therapeutic moiety is on the inside of said liposome.

18. The lung and/or heart-specific therapeutic complex of claim 10, wherein said therapeutic moiety is an enzyme which cleaves a prodrug.

19. The lung and/or heart-specific therapeutic complex of claim 10, wherein said linker is cleavable by an enzyme.

20. The lung and/or heart-specific therapeutic complex of claim 10, wherein said therapeutic moiety is an antibiotic.

21. The lung and/or heart-specific therapeutic complex of claim 10, wherein said therapeutic moiety is a chemotherapeutic agent.

22. A method of determining the presence or concentration of carbonic anhydrase IV in a tissue or cell, comprising administering the therapeutic complex of claim 10 to said tissue or cell in vitro or in vivo, and identifying or quantitating the amount of the therapeutic complex which bound.

23. A pharmaceutical composition comprising the lung and/or heart-specific therapeutic complex of claim 10 and one or more pharmaceutically acceptable carriers.

24. A lung and/or kidney-specific therapeutic complex which interacts with a targeted endothelial cell, comprising:
a ligand which attaches said therapeutic complex to the luminal surface of a vascular endothelial cell membrane of the specific tissue, wherein the ligand binds to SEQ ID NO:4 or 6, or a homolog thereof;
a linker; and
a therapeutic moiety, wherein said linker links the ligand with the therapeutic moiety.

25. The lung and/or kidney-specific therapeutic complex of claim 24, wherein said ligand is an antibody or a binding part thereof.

26. The lung and/or kidney-specific therapeutic complex of claim 24, wherein said ligand does not activate a receptor.

27. The lung and/or kidney-specific therapeutic complex of claim 24, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

28. The lung and/or kidney-specific therapeutic complex of claim 24, wherein said linker is pH sensitive.

29. The lung and/or kidney-specific therapeutic complex of claim 28, wherein said pH sensitive linker is an acid sensitive bond between the ligand and the therapeutic moiety.

30. The lung and/or kidney-specific therapeutic complex of claim 24, wherein said linker is a liposome.

31. The lung and/or kidney-specific therapeutic complex of claim 30, wherein said ligand is on the outside of the liposome and said therapeutic moiety is on the inside of said liposome.

32. The lung and/or kidney-specific therapeutic complex of claim 24, wherein said therapeutic moiety is an enzyme which cleaves a prodrug.

33. The lung and/or kidney-specific therapeutic complex of claim 24, wherein said linker is cleavable by an enzyme.

34. The lung and/or kidney-specific therapeutic complex of claim 27, wherein said at least one pharmaceutical is an immunosuppressant.

35. The lung and/or kidney-specific therapeutic complex of claim 27, wherein said at least one pharmaceutical is an antithrombotic.

36. A method of determining the presence or concentration of dipeptidyl peptidase IV in a tissue or cell, comprising administering the therapeutic complex of claim 24 to said tissue or cell in vitro or in vivo, and identifying or quantitating the amount of the therapeutic complex which bound.

37. A pharmaceutical composition comprising the lung and/or kidney-specific therapeutic complex of claim 24 and one or more pharmaceutically acceptable carriers.

38. A pancreatic and/or gut-specific therapeutic complex which interacts with a targeted endothelial cell, comprising:
a ligand which attaches said therapeutic complex to the luminal surface of a vascular endothelial cell membrane of the specific tissue, wherein said ligand binds to SEQ ID NO:14 or 16, or a homolog thereof;
a linker; and
a therapeutic moiety, wherein said linker links the ligand with the therapeutic moiety.

39. The pancreatic and/or gut-specific therapeutic complex of claim 38, wherein said ligand is an antibody or binding part thereof.

40. The pancreatic and/or gut-specific therapeutic complex of claim 38, wherein said ligand does not activate a receptor.

41. The pancreatic and/or gut-specific therapeutic complex of claim 38, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

42. The pancreatic and/or gut-specific therapeutic complex of claim 38, wherein said linker is pH sensitive.

43. The pancreatic and/or gut-specific therapeutic complex of claim 42, wherein said pH sensitive linker is an acid sensitive bond between the ligand and the therapeutic moiety.

44. The pancreatic and/or gut-specific therapeutic complex of claim 38, wherein said linker is a liposome.

45. The pancreatic and/or gut-specific therapeutic complex of claim 44, wherein said ligand is on the outside of the liposome and said therapeutic moiety is on the inside of said liposome.

46. The pancreatic and/or gut-specific therapeutic complex of claim 38, wherein said therapeutic moiety is an enzyme which cleaves a prodrug.

47. The pancreatic and/or gut-specific therapeutic complex of claim 38, wherein said linker is cleavable by an enzyme.

48. The pancreatic and/or gut-specific therapeutic complex of claim 41 wherein said at least one pharmaceutical is an antibiotic or an antiviral.

49. The pancreatic and/or gut-specific therapeutic complex of claim 41 wherein said at least one pharmaceutical is an antithrombotic.

50. A method of determining the presence or concentration of ZG16-p in a tissue or cell, comprising administering the therapeutic complex of claim 38 to said tissue or cell in vitro or in vivo, and identifying or quantitating the amount of the therapeutic complex which bound.

51. A pharmaceutical composition comprising the pancreatic and/or gut-specific therapeutic complex of claim 38 and one or more pharmaceutically acceptable carriers.

52. A prostate-specific therapeutic complex which interacts with a targeted endothelial cell, comprising:
a ligand which attaches said therapeutic complex to the luminal surface of a vascular endothelial cell membrane of the specific tissue comprising SEQ ID NO:23 or a homolog thereof;
a linker; and
a therapeutic moiety, wherein said linker links the ligand with the therapeutic moiety.

53. The prostate-specific therapeutic complex of claim 52, wherein said ligand is an antibody or a binding part thereof.

54. The prostate-specific therapeutic complex of claim 52, wherein said ligand does not activate a receptor.

55. The prostate-specific therapeutic complex of claim 52, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

56. The prostate-specific therapeutic complex of claim 52, wherein said linker is pH sensitive.

57. The prostate-specific therapeutic complex of claim 56, wherein said pH sensitive linker is an acid sensitive bond between the ligand and the therapeutic moiety.

58. The prostate-specific therapeutic complex of claim 52, wherein said linker is a liposome.

59. The prostate-specific therapeutic complex of claim 58, wherein said ligand is on the outside of the liposome and said therapeutic moiety is on the inside of said liposome.

60. The prostate-specific therapeutic complex of claim 52, wherein said therapeutic moiety is an enzyme which cleaves a prodrug.

61. The prostate-specific therapeutic complex of claim 52, wherein said linker is cleavable by an enzyme.

62. The prostate-specific therapeutic complex of claim 55, wherein said at least one pharmaceutical is an immunosuppressant.

63. The prostate-specific therapeutic complex of claim 52, wherein said therapeutic moiety is a chemotherapeutic.

64. A method of determining the presence or concentration of Albumin fragment in a tissue or cell, comprising administering the therapeutic complex of claim 52 to said tissue or cell in vitro or in vivo, and identifying or quantitating the amount of the therapeutic complex which bound.

65. A pharmaceutical composition comprising the prostate-specific therapeutic complex of claim 52 and one or more pharmaceutically acceptable carriers.

66. A brain-specific therapeutic complex which interacts with a targeted endothelial cell, comprising:
a ligand which attaches said therapeutic complex to the luminal surface of a vascular endothelial cell membrane of the specific tissue wherein said ligand binds to SEQ ID NO:26 or 28 or a homolog thereof;
a linker; and
a therapeutic moiety, wherein said linker links the ligand with the therapeutic moiety.

67. The brain-specific therapeutic complex of claim 66, wherein said ligand is an antibody or a binding part thereof.

68. The brain-specific therapeutic complex of claim 66, wherein said ligand does not activate a receptor.

69. The brain-specific therapeutic complex of claim 66, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

70. The brain-specific therapeutic complex of claim 66, wherein said linker is pH sensitive.

71. The brain-specific therapeutic complex of claim 70, wherein said pH sensitive linker is an acid sensitive bond between the ligand and the therapeutic moiety.

72. The brain-specific therapeutic complex of claim 66, wherein said linker is a liposome.

73. The brain-specific therapeutic complex of claim 72, wherein said ligand is on the outside of the liposome and said therapeutic moiety is on the inside of said liposome.

74. The brain-specific therapeutic complex of claim 66, wherein said therapeutic moiety is an enzyme which cleaves a prodrug.

75. The brain-specific therapeutic complex of claim 66, wherein said linker is cleavable by an enzyme.

76. The brain-specific therapeutic complex of claim 69, wherein said at least one pharmaceutical is an immunosuppressant.

77. The brain-specific therapeutic complex of claim 69, wherein said at least one pharmaceutical is an antithrombotic.

78. A method of determining the presence or concentration of CD71 (transferrin receptor) in a tissue or cell, comprising administering the therapeutic complex of claim 66 to said tissue or cell in vitro or in vivo, and identifying or quantitating the amount of the therapeutic complex which bound.

79. A pharmaceutical composition comprising the brain-specific therapeutic complex of claim 66 and one or more pharmaceutically acceptable carriers.

80. A pancreas and/or gut-specific therapeutic complex which interacts with a targeted endothelial cell, comprising:
a ligand which attaches said therapeutic complex to the luminal surface of a vascular endothelial cell membrane of the specific tissue wherein said ligand binds to SEQ ID NO:18 or 20, or a homolog thereof;
a linker; and
a therapeutic moiety, wherein said linker links the ligand with the therapeutic moiety.

81. The pancreas and/or gut-specific therapeutic complex of claim 80, wherein said ligand is an antibody or a binding part thereof.

82. The pancreas and/or gut-specific therapeutic complex of claim 80, wherein said ligand does not activate a receptor.

83. The pancreas and/or gut-specific therapeutic complex of claim 80, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

84. The pancreas and/or gut-specific therapeutic complex of claim 80, wherein said linker is pH sensitive.

85. The pancreas and/or gut-specific therapeutic complex of claim 84, wherein said pH sensitive linker is an acid sensitive bond between the ligand and the therapeutic moiety.

86. The pancreas and/or gut-specific therapeutic complex of claim 80, wherein said linker is a liposome.

87. The pancreas and/or gut-specific therapeutic complex of claim 86, wherein said ligand is on the outside of the liposome and said therapeutic moiety is on the inside of said liposome.

88. The pancreas and/or gut-specific therapeutic complex of claim 80, wherein said therapeutic moiety is an enzyme which cleaves a prodrug.

89. The pancreas and/or gut-specific therapeutic complex of claim 80, wherein said linker is cleavable by an enzyme.

90. The pancreas and/or gut-specific therapeutic complex of claim 83, wherein said at least one pharmaceutical is an immunosuppressant.

91. The pancreas and/or gut-specific therapeutic complex of claim 83, wherein said at least one pharmaceutical is an antithrombotic.

92. A method of determining the presence or concentration of MAdCAM in a tissue or cell, comprising administering the therapeutic complex of claim 80 to said tissue or cell in vitro or in vivo, and identifying or quantitating the amount of the therapeutic complex which bound.

93. A pharmaceutical composition comprising the pancreas and/or gut-specific therapeutic complex of claim 80 and one or more pharmaceutically acceptable carriers.

94. A kidney-specific therapeutic complex which interacts with a targeted endothelial cell, comprising:
a ligand which attaches said therapeutic complex to the luminal surface of a vascular endothelial cell membrane of the specific tissue, wherein said ligand binds to SEQ ID NO:30 or 32, or a homolog thereof;
a linker; and
a therapeutic moiety, wherein said linker links the ligand to the therapeutic moiety.

95. The kidney-specific therapeutic complex of claim 94, wherein said ligand is an antibody or a binding part thereof.

96. The kidney-specific therapeutic complex of claim 94, wherein said ligand does not activate a receptor.

97. The kidney-specific therapeutic complex of claim 94, wherein said therapeutic moiety is selected from the group consisting of at least one pharmaceutical, at least one gene, at least one antisense oligonucleotide, at least one chemotherapeutic agent, at least one contrast agent, at least one protein, at least one toxin, at least one radioactive atom, and a mixture thereof.

98. The kidney-specific therapeutic complex of claim 94, wherein said linker is pH sensitive.

99. The kidney-specific therapeutic complex of claim 98, wherein said pH sensitive linker is an acid sensitive bond between the ligand and the therapeutic moiety.

100. The kidney-specific therapeutic complex of claim 94, wherein said linker is a liposome.

101. The kidney-specific therapeutic complex of claim 100, wherein said ligand is on the outside of the liposome and said therapeutic moiety is on the inside of said liposome.

102. The kidney-specific therapeutic complex of claim 94, wherein said therapeutic moiety is an enzyme which cleaves a prodrug.

103. The kidney-specific therapeutic complex of claim 94, wherein said linker is cleavable by an enzyme.

104. The kidney-specific therapeutic complex of claim 97, wherein said at least one pharmaceutical is a chemotherapeutic.

105. A method of determining the presence or concentration of CD90 (Thy-1) in a tissue or cell, comprising administering the therapeutic complex of claim 94 to said tissue or cell in vitro or in vivo, and identifying or quantitating the amount of the therapeutic complex which bound.

106. A pharmaceutical composition comprising the kidney-specific therapeutic complex of claim 94 and one or more pharmaceutically acceptable carriers.

107. A method for the treatment of prostate cancer comprising
administering a prostate-specific therapeutic complex of claim 52 in an amount effective to reduce the number of cancer cells, wherein said therapeutic moiety is a chemotherapeutic agent.

108. The method of claim 107 wherein said chemotherapeutic agent is selected from the group consisting of an antisense RNA, an apoptosis-inducing protein, a nucleotide analog, a radioactive molecule, a toxin, and any other chemotherapeutic agent.

109. A method for the treatment of brain tumors comprising
administering a brain-specific therapeutic complex of claim 66 in an amount effective to reduce the number of cancer cells, wherein said therapeutic moiety is a chemotherapeutic agent.

110. The method of claim 109 wherein said chemotherapeutic agent is selected from the group consisting of an antisense RNA, an apoptosis-inducing protein, a nucleotide analog, a radioactive molecule, a toxin, and any other chemotherapeutic agent.

111. A method for the treatment of pancreatic cancer comprising
administering the pancreas and/or gut-specific therapeutic complex of claim 38 in an amount effective to reduce the amount of thrombosis, wherein said therapeutic moiety is an antithrombotic agent.

112. A method for the treatment of pancreatic cancer comprising
administering the pancreas and/or gut-specific therapeutic complex of claim 80 in an amount effective to reduce the amount of thrombosis, wherein said therapeutic moiety is an antithrombotic agent.

113. A method for the treatment of kidney transplant rejection comprising
administering the kidney and/or lung specific therapeutic complex of claim 94 in an amount sufficient to reduce the rejection of the kidney transplant, wherein said therapeutic moiety is an immunosuppressant agent.

114. The method of claim 113 wherein said immunosuppressant agent is a corticosteroid or a cyclosporin.

115. A method for delivering a therapeutic agent to a specific tissue, comprising:
administering a therapeutically effective amount of a therapeutic complex, said therapeutic complex comprising: a ligand which binds to a tissue-specific luminally expressed protein, a therapeutic moiety, and a linker which links said therapeutic moiety to said ligand, wherein said tissue-specific luminally expressed protein is selected from the group consisting of CD71, CD90, MAdCAM, Albumin fragment, carbonic anhydrase IV, ZG16-p and dipeptidyl peptidase IV.

116. A method for lung and/or heart-specific delivery of a substance in vivo or in vitro, comprising:
providing a carbonic anhydrase IV-binding agent, and
administering said carbonic anhydrase IV-binding agent in vivo or in vitro,
wherein said substance is delivered to the lung and/or heart or lung and/or heart tissue as a result of the administration of the carbonic anhydrase IV-binding agent.

117. The method of claim 116, wherein said carbonic anhydrase IV-binding agent is selected from the group consisting of an antibody, a protein, a peptide, an oligonucleotide, a small molecule, and a polysaccharide.

118. The method of claim 116, wherein said substance is covalently or non-covalently bound to said carbonic anhydrase IV-binding agent.

119. The method of claim 116, wherein said substance is administered separately from said carbonic anhydrase IV-binding agent.

120. The method of claim 116, wherein said substance is selected from the group consisting of a therapeutic agent, a contrast agent, a diagnostic agent, and a toxic agent.

121. The method of claim 116, wherein said substance is said carbonic anhydrase IV-binding agent.

122. The method of claim 116, wherein said in vivo administration is by a method selected from the group consisting of injection, oral delivery, aerosolization, an implantable pump, a patch, and a stent.

123. The method of claim 116, wherein said in vitro administration is to a lung and/or heart or lung and/or heart tissue to be transplanted.

124. A method of identifying a lung and/or heart-specific ligand, comprising:
identifying a carbonic anhydrase IV-binding agent.

125. The method of claim 124 wherein said identification is by a method selected from the group consisting of antibody production, combinatorial library screening, one-hybrid technology, molecular modeling and two-hybrid technology.

126. A method for brain-specific delivery of a substance in vivo or in vitro, comprising:
providing a CD71 (transferrin receptor)-binding agent, and
administering said CD71-binding agent in vivo or in vitro, wherein said substance is delivered to the brain or brain tissue as a result of the administration of the CD71-binding agent.

127. The method of claim 126, wherein said CD71-binding agent is selected from the group consisting of an antibody, a protein, a peptide, an oligonucleotide, a small molecule, and a polysaccharide.

128. The method of claim 126, wherein said substance is covalently or non-covalently bound to said CD71-binding agent.

129. The method of claim 126, wherein said substance is administered separately from said CD71-binding agent.

130. The method of claim 126, wherein said substance is selected from the group consisting of a therapeutic agent, a contrast agent, a diagnostic agent, and a toxic agent.

131. The method of claim 126, wherein said substance is said CD71-binding agent.

132. The method of claim 126, wherein said in vivo administration is by a method selected from the group consisting of injection, oral delivery, aerosolization, an implantable pump, a patch, and a stent.

133. The method of claim 126, wherein said in vitro administration is to a brain or brain tissue to be transplanted.

134. A method of identifying a brain-specific ligand, comprising:
identifying a CD71-binding agent.

135. The method of claim 134 wherein said identification is by a method selected from the group consisting of antibody production, combinatorial library screening, one-hybrid technology, molecular modeling and two-hybrid technology.

136. A method for kidney-specific delivery of a substance in vivo or in vitro, comprising:
providing a CD90(Thy-1)-binding agent, and
administering said CD90-binding agent in vivo or in vitro, wherein said substance is delivered to the kidney or kidney tissue as a result of the administration of the CD90-binding agent.

137. The method of claim 136, wherein said CD90-binding agent is selected from the group consisting of an antibody, a protein, a peptide, an oligonucleotide, a small molecule, and a polysaccharide.

138. The method of claim 136, wherein said substance is covalently or non-covalently bound to said CD90-binding agent.

139. The method of claim 136, wherein said substance is administered separately from said CD90-binding agent.

140. The method of claim 136, wherein said substance is selected from the group consisting of a therapeutic agent, a contrast agent, a diagnostic agent, and a toxic agent.

141. The method of claim 136, wherein said substance is said CD90-binding agent.

142. The method of claim 136, wherein said in vivo administration is by a method selected from the group consisting of injection, oral delivery, aerosolization, an implantable pump, a patch, and a stent.

143. The method of claim 136, wherein said in vitro administration is to a kidney or kidney tissue to be transplanted.

144. A method of identifying a kidney-specific ligand, comprising:
identifying a CD90-binding agent.

145. The method of claim 144 wherein said identification is by a method selected from the group consisting of antibody production, combinatorial library screening, one-hybrid technology, molecular modeling and two-hybrid technology.

146. A method for lung and/or kidney-specific delivery of a substance in vivo or in vitro, comprising:
providing a dipeptidyl peptidase IV-binding agent, and
administering said dipeptidyl peptidase IV-binding agent in vivo or in vitro,
wherein said substance is delivered to the lung and/or kidney or lung and/or kidney tissue as a result of the administration of the dipeptidyl peptidase IV-binding agent.

147. The method of claim 146, wherein said dipeptidyl peptidase IV-binding agent is selected from the group consisting of an antibody, a protein, a peptide, an oligonucleotide, a small molecule, and a polysaccharide.

148. The method of claim 146, wherein said substance is covalently or non-covalently bound to said dipeptidyl peptidase IV-binding agent.

149. The method of claim 146, wherein said substance is administered separately from said dipeptidyl peptidase IV-binding agent.

150. The method of claim 146, wherein said substance is selected from the group consisting of a therapeutic agent, a contrast agent, a diagnostic agent, and a toxic agent.

151. The method of claim 146, wherein said substance is said dipeptidyl peptidase IV-binding agent.

152. The method of claim 146, wherein said in vivo administration is by a method selected from the group consisting of injection, oral delivery, aerosolization, an implantable pump, a patch, and a stent.

153. The method of claim 146, wherein said in vitro administration is to a lung and/or kidney or lung and/or kidney tissue to be transplanted.

154. A method of identifying a lung and/or kidney-specific ligand, comprising:
identifying a dipeptidyl peptidase IV-binding agent.

155. The method of claim 154 wherein said identification is by a method selected from the group consisting of antibody production, combinatorial library screening, one-hybrid technology, molecular modeling and two-hybrid technology.

156. A method for pancreas and/or gut-specific delivery of a substance in vivo or in vitro, comprising:
providing a ZG16-p-binding agent, and
administering said ZG16-p-binding agent in vivo or in vitro, wherein said substance is delivered to the pancreas and/or gut or pancreas and/or gut tissue as a result of the administration of the ZG16-p-binding agent.

157. The method of claim 156, wherein said ZG16-p-binding agent is selected from the group consisting of an antibody, a protein, a peptide, an oligonucleotide, a small molecule, and a polysaccharide.

158. The method of claim 156, wherein said substance is covalently or non-covalently bound to said ZG16-p-binding agent.

159. The method of claim 156, wherein said substance is administered separately from said ZG16-p-binding agent.

160. The method of claim 156, wherein said substance is selected from the group consisting of a therapeutic agent, a contrast agent, a diagnostic agent, and a toxic agent.

161. The method of claim 156, wherein said substance is said ZG16-p-binding agent.

162. The method of claim 156, wherein said in vivo administration is by a method selected from the group consisting of injection, oral delivery, aerosolization, an implantable pump, a patch, and a stent.

163. The method of claim 156, wherein said in vitro administration is to a pancreas and/or gut or pancreas and/or gut tissue to be transplanted.

164. A method of identifying a pancreas and/or gut-specific ligand, comprising:
identifying a ZG16-p-binding agent.

165. The method of claim 164 wherein said identification is by a method selected from the group consisting of antibody production, combinatorial library screening, one-hybrid technology, molecular modeling and two-hybrid technology.

166. A method for pancreas and/or gut-specific delivery of a substance in vivo or in vitro, comprising:
providing a MAdCAM-binding agent, and
administering said MAdCAM-binding agent in vivo or in vitro, wherein said substance is delivered to the pancreas and/or gut or pancreas and/or gut tissue as a result of the administration of the MAdCAM-binding agent.

167. The method of claim 166, wherein said MAdCAM-binding agent is selected from the group consisting of an antibody, a protein, a peptide, an oligonucleotide, a small molecule, and a polysaccharide.

168. The method of claim 166, wherein said substance is covalently or non-covalently bound to said MAdCAM-binding agent.

169. The method of claim 166, wherein said substance is administered separately from said MAdCAM-binding agent.

170. The method of claim 166, wherein said substance is selected from the group consisting of a therapeutic agent, a contrast agent, a diagnostic agent, and a toxic agent.

171. The method of claim 166, wherein said substance is said MAdCAM-binding agent.

172. The method of claim 166, wherein said in vivo administration is by a method selected from the group consisting of injection, oral delivery, aerosolization, an implantable pump, a patch, and a stent.

173. The method of claim 166, wherein said in vitro administration is to a pancreas and/or gut or pancreas and/or gut tissue to be transplanted.

174. A method of identifying a pancreas and/or gut-specific ligand, comprising:
identifying a MAdCAM-binding agent.

175. The method of claim 174 wherein said identification is by a method selected from the group consisting of antibody production, combinatorial library screening, one-hybrid technology, molecular modeling and two-hybrid technology.

176. A method for prostate-specific delivery of a substance in vivo or in vitro, comprising:
providing a Albumin fragment-binding agent, and
administering said Albumin fragment-binding agent in vivo or in vitro,
wherein said substance is delivered to the prostate or prostate tissue as a result of the administration of the Albumin fragment-binding agent.

177. The method of claim 176, wherein said Albumin fragment-binding agent is selected from the group consisting of an antibody, a protein, a peptide, an oligonucleotide, a small molecule, and a polysaccharide.

178. The method of claim 176, wherein said substance is covalently or non-covalently bound to said Albumin fragment-binding agent.

179. The method of claim 176, wherein said substance is administered separately from said Albumin fragment-binding agent.

180. The method of claim 176, wherein said substance is selected from the group consisting of a therapeutic agent, a contrast agent, a diagnostic agent, and a toxic agent.

181. The method of claim 176, wherein said substance is said Albumin fragment-binding agent.

182. The method of claim 176, wherein said in vivo administration is by a method selected from the group consisting of injection, oral delivery, aerosolization, an implantable pump, a patch, and a stent.

183. The method of claim 176, wherein said in vitro administration is to a prostate or prostate tissue to be transplanted.

184. A method of identifying a prostate-specific ligand, comprising:
identifying a Albumin fragment-binding agent.

185. The method of claim 184 wherein said identification is by a method selected from the group consisting of antibody production, combinatorial library screening, one-hybrid technology, molecular modeling and two-hybrid technology.


SEQUENCE LISTING (33 sequences)

SEQ ID NO: 1 - protein, 10 aa, Artificial Sequence
  Proteinase cleavable peptide linker.
GFPRGFPAGG

SEQ ID NO: 2 - protein, 12 aa, Artificial Sequence
  Turin protease cleavable peptide linker.
TRHRQPRGWEQL

SEQ ID NO: 3 - protein, 5 aa, Artificial Sequence
  Peptide corresponding to rat DPP4.
FRPAE

SEQ ID NO: 4 - protein, 767 aa, Rattus norvegicus
MKTPWKVLLGLLGVAALVTIITVPVVLLNKDEAAADSARTYTLADYLKNTFRVKSYSLRWVSDSEYLYKQENNILLFNAEHGNSSIFLENSTFEIFGDSISDYSVSPDRLFVLLEYNYVKQWRHSYTASYSIYDLNKRQLITEEKIPNNTQWITWSQEGHKLAYVWKNDIYVKIEPHLPSHRITSTGKENVIFNGINDWVYEEEIFGAYSALWWSPNGTFLAYAQFNDTGVPLIEYSFYSDESLQYPKTVWIPYPKAGAVNPTVKFFIVNTDSLSSTTTTIPMQITAPASVTTGDHYLCDVAWVSEDRISLQWLRRIQNYSVMAICDYDKTTLVWNCPTTQEHIETSATGWCGRFRPAEPHFTSDGSSFYKIVSDKDGYKHICQFQKDRKPEQVCTFITKGAWEVISIEALTSDYLYYISNEYKEMPGGRNLYKIQLTDHTNKKCLSCDLNPERCQYYSVSLSKEAKYYGLGCRGPGLPLYTLHRSTDQKELRVLEDNSALDKMLQDVQMPSKKLDFIVLNETRFWYQMILPPHFDKSKKYPLLIDVYAGPCSQKADAAFRLNWATYLASTENIIVASFDGRGSGYQGDKIMHAINKRLGTLEVEDQIEAARQFLKMGFVDSKRVAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDAGVDFQAMWYTDEDHGIASSTAHQHIYSHMSHFLQQCFSLR

SEQ ID NO: 5 - DNA, 4835 bp, Rattus norvegicus
gagcagaggcgcaggacgtccgtctccgcgcgcgtgacttctgcctgcgctcaagcttcagagttcagtttcaaggagccgcccaaccatgaagacaccgtggaaggttcttctgggactgcttggtgtcgctgcgcttgtcaccatcatcaccgtgccagtggttctgctgaacaaagatgaagcggccgctgatagcgcgagaacttacacactagctgactatttaaagaatacctttcgggtcaagtcctactccttgcggtgggtttcagattctgaatacctctacaagcaagaaaacaatatcttgctattcaatgctgaacacgggaacagctccattttcttggagaacagtacctttgagatctttggagattctataagtgattattcagtgtcacccgacagactgttcgttctcttagaatacaattatgtgaagcaatggagacactcctacacggcttcatacagtatttatgacttgaataaaagacagctgatcacagaagagaagattccaaataatacacagtggatcacatggtcacaagaaggtcacaaattggcatatgtctggaagaatgatatttatgttaaaattgaaccacatttgcctagtcataggatcacatcaacaggaaaagaaaatgtaatatttaacggaataaatgactgggtttatgaagaggaaatcttcggtgcctactccgcactgtggtggtctccaaacggcacttttctagcttatgcccagtttaacgacaccggagtgcctctcattgaatactccttctactctgatgagtcactgcagtaccccaagacagtctggattccgtacccaaaggcaggagctgtgaatccaactgtaaagttctttattgtaaatacagactctctcagctcaactactactacgattcccatgcaaatcaccgctcctgcatctgtgacaacaggggatcactacttgtgtgacgtggcctgggtttcagaagacagaatctcgttgcagtggctcaggaggattcagaactattccgtgatggcgatctgcgactatgataagaccaccctagtatggaactgtccaacgacgcaggagcatattgaaacgagtgccacaggctggtgcggaagatttaggcctgcagaaccccacttcacctccgacggaagcagcttctataaaatcgtcagtgacaaagatggctacaaacacatctgccagttccagaaagataggaaacccgaacaggtctgtacatttattacaaaaggagcctgggaagtcattagtatcgaagctctgaccagcgattatctgtactacattagtaatgaatataaagaaatgccaggaggaagaaatctttataaaattcagcttactgaccacacaaataagaagtgccttagttgtgacctgaatccagaaagatgccagtattactcggtgtcacttagtaaagaggcaaagtactatcagctgggatgccggggccctggtctgcccctctacactctgcatcgcagcactgatcaaaaagagctgagagtcctggaggacaattctgctttggataaaatgctgcaagatgtccaaatgccttcaaaaaaattggacttcattgttctgaatgaaacaagattttggtatcaaatgatcttacctcctcattttgataaatccaagaaataccctctactaatagatgtatatgcaggtccctgtagtcaaaaagcagatgctgccttcagactcaactgggccacttaccttgcaagcacagaaaacatcatagtagctagctttgatggcagaggaagtggttaccaaggagataagatcatgcatgcaatcaacaaaagacttggaacactggaagttgaagatcaaattgaagcagccaggcaatttttaaaaatgggatttgtggacagcaagcgagttgcaatttggggctggtcatatggagggtacgtaacctcaatggtcctgggatcgggaagtggcgtgttcaagtgtggaatagccgtggcgcccgtgtcacggtgggagtactatgactcagtatacacagagcgttacatgggtctcccaactccagaggacaaccttgaccattacaggaactcaacagtcatgagcagagctgaaaattttaagcaagttgagtacctccttattcacggtacagcagatgataatgttcactttcagcagtcagctcagatctccaaagccctggtggatgctggcgtggatttccaagcaatgtggtacacggacgaagaccatgggatcgccagcagcacagctcaccagcacatctattcccacatgagccatttcctccagcagtgcttctccttacgctagcatggcaaggctctccgcagcttactcaagagcacacttgtcctcattatctcaaaactgcactgttaagatgacgattttaataatgtcgcctcgagaaattccagcctacttcccagttttatacctgcaatcctaactaaggatgcctgtcttcagaacagattattaccttacagcaatttggatttccccctctgttttgtttatcatttaaaaccatttccacatcagctgctgaaacaacaaatataaattatttttgcaagagctatgcatagatttcctgagcagaatttcaatttttttcccccttactaggctggtccaaatcttgttcccttatttaagggggtggcaagacgtgggtaatgatgtcattaggccagcaacaagagaagcgggaacagagaatatggctagaaacccaggtccaagcatacaaacccaaccaggctactgtcagctcgcctcgagaagagctgctcactgccagactggcaccgttttctgagaaagactattcaaacagtctcaggaaatcatatatgcaaagcactgacttctaagtaaaaccacagcagttgaatagactccaaagaaatgcaagggacgctgccagcaatgtaagggccccaggtgccagttatggctataggtgctacataaacacagcaagcctgatgggaaagcatgttaaatgtgcttttaaaaattaccaagtctcctagtgagaagaggcagcttggaacatagcgacttgccccgttaaaagttgaaaatatttgtgtcacaaattctaacatgaaggaatacttgcgtcagttcttcctacttcctttctttgagcattttcattaaagcattttaacttcattatctttctaatggaaaactgtatgagaatgttttgtgttattatttctattctacacactggaatgttgcctggtcatttagcaagtatgcttccattttttcaaaggtaatgggttatatcttgaatcaaacttaaactgcattgacatatggacacatttgttcaaaggttcttgtttaacttgtgtgaaatccaagactgtcttgtaaacatggaaagagttcaacttttaaaaaaaaatttagatacataaaactgtttaaagttatatgattcataagagtttatctaatacccccagaaatttctactcacatttatcacatagcttggtcatttacatactatggaactcataatattatttaacttaggggagcacgtgaggttcgtggcacgagatggaatgctatcagcagagtagacatgtttttccagggtcttgttttttgtttttgtttctggtctcttcctgtttgggcggagggtaatataatagataatatacataatagaatacactctgatacctgacttagccgtgttttgacaacttggaaacttgattcaattatttataacacagctgaaaatttaaaatggactccacacatttaaatgcagtttcaggccaattttctaggtacaattaccacagacaggtgagctacagcataaattccaaacatggcagaaatggaaattacctataaatataaatgagtttagatattgatgagcctgatgctatttcccgggcactccactgttcccctcaccttaaggaactctcaagtcctgctcttccactgcaagcacagctggtccttaaatctacaggcctctggctacagtccgaatttgaacacagttctgtcaccgtgtgcagcagcagcagccatgtgcaaagttctagatcaaggaacaaaggtagcacatgttcctgacagtgtggaaacataaacataaatgcgaattaaatagaaattatcccttctgaattctttttgttcctttcatttctaaataggttgttcctggagcctgaattaataaaaagaacacagcacacatttttcaggcgatgagggtttcacatggtgataatgtgaatacattcagtttttatttgattctcataggtcaagttttactgttcggtaagagttgtaaattagattaaaaccctgatgcataagttgtaaacaaacttaatttaagagcaagtttgaaaagcacaagagctaataacaccactgaggcatatagacaagtctcttatgggcatatgcagctccctgaagcgcatggatcaagctaccgcctcagagcacaccagcaccaggggcgcatgctaaaggaagagctcccctccccaccccccatgcttcacgatccatgttgacttcagtctgtgccattctgggcatcatagttctccttcagattattagcagttccacctcttggcacgtactacttttgctctaagttggagtgagagtactggtttataagattactggatttgtacaatatttaagattcaataaattctaagtg

SEQ ID NO: 6 - DNA, 3407 bp, Homo sapiens
  CDS (76)...(2376)
cgcgcgtctccgccgcccgcgtgacttctgcctgcgctccttctctgaacgctcacttccgaggagacgccgacgatgaagacaccgtggaagattcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttattgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcaccacgagactggcacagttttctgagaaagactattcaaacagtctcaggaaatcaaatatcgaaagcactgacttctaagtaaaccacagcagttgaaagactccaaagaaatgtaagggaaactgccagcaacgcagcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttc

SEQ ID NO: 7 - protein, 766 aa, Homo sapiens [sequence omitted]

SEQ ID NO: 8 - protein, 9 aa, Artificial Sequence
  Peptide corresponding to rat carbonic anhydrase IV.
DSHWCYEIQ

SEQ ID NO: 9 - protein, 309 aa, Rattus norvegicus
MQLLLALLALAYVAPSTEDSHWCYEIQAKEPNSHCSGPEQWTGDCKKNQQSPINIVTSKTKLNPSLTPFTFVGYDQKKKWEVKNNQHSVEMSLGEDIYIFGGDLPTQYKAIQLHLHWSEESNKGSEHSIDGKHFAMEMHVVHKKMTTGDKVQDSDSKDKIAVLAFMVEVGNEVNEGFQPLVEALSRLSKPFTNSTVSESCLQDMLPEKKKLSAYFRYQGSLTTPGCDETVIWTVFEEPIKIHKDQFLEFSKKLYYDQEQKLNMKDNVRPLQPLGNRQVFRSHASGRLLSLPLPTLLVPTLTCLVASFLH

SEQ ID NO: 10 - DNA, 1205 bp, Rattus norvegicus
ggcttcgttggtgctggaccccaggctgggcagcgtctatgccctcaagcaccatgcagctccttcttgctctactggcgctggcttacgtggccccctctactgaagattcacactggtgctatgagattcaagccaaggagcccaacagccattgctcagggcctgaacaatggactggagactgtaagaagaaccagcagtctcctatcaatattgtcactagtaagacaaagttgaaccccagtctgacacccttcactttcgttggctatgaccaaaagaagaagtgggaagttaagaacaaccaacactcagtggaaatgtcgctgggggaggacatctatatttttggaggagatctgcccacccagtacaaggccatacagttacacctgcactggtcagaggagtcgaacaagggttcagagcacagtattgatgggaaacattttgccatggagatgcatgtcgtgcataagaagatgacaacaggcgataaggtgcaggactcggactccaaggacaagattgcggtgctggcattcatggttgaggtgggaaacgaggtaaacgagggcttccagcccctggtggaggcactgtccaggctctccaaaccctttacaaactccacagtgagtgagagctgcctgcaggatatgcttcctgaaaagaagaaactgtctgcctacttccgttaccagggctcactgactacaccaggctgtgatgagactgtcatctggactgtgttcgaggaacctattaagatccataaagaccagttcctggaattctcaaaaaagctctactatgaccaagaacagaagttgaacatgaaggacaatgtgaggcccctgcagccactgggaaaccgccaggtgttcaggtctcatgcctcaggacgactgctgtctttgcccctgcccactctattggtccccacactcacctgcctggtggccagcttcctccactgatggtcaaattctggatatctggcctctgacctcaacctttagaggatatggcttctctttctcaatctttccaggttgaactttggggactattaaggtgtgctgtggtggtgcacacctttaatcccagccctggatggagagagcagagacctatggatctctgaccttcccctaacccctgattaaattaaataaaatatggatatgtttttactctta

SEQ ID NO: 11 - protein, 312 aa, Homo sapiens
MRMLLALLALSAARPSASAESHWCYEVQAESSNYPCLVPVKWGGNCQKDRQSPINIVTTKAKVDKKLGRFFFSGYDKKQTWTVQNNGHSVMMLLENKASISGGGLPAPYQAKQLHLHWSDLPYKGSEHSLDGEHFAMEMHIVHEKEKGTSRNVKEAQDPEDEIAVLAFLVEAGTQVNEGFQPLVEALSNIPKPEMSTTMAESSLLDLLPKEEKLRHYFRYLGSLTTPTCDEKVVWTVFREPIQLHREQILAFSQKLYYDKEQTVSMKDNVRPLQQLGQRTVIKSGAPGRPLPWALPALLGPMLACLLAGFLR

SEQ ID NO: 12 - DNA, 1104 bp, Homo sapiens
ctcggtgcgcgaccccggctcagaggactctttgctgtcccgcaagatgcggatgctgctggcgctcctggccctctccgcggcgcggccatcggccagtgcagagtcacactggtgctacgaggttcaagccgagtcctccaactacccctgcttggtgccagtcaagtggggtggaaactgccagaaggaccgccagtcccccatcaacatcgtcaccaccaaggcaaaggtggacaaaaaactgggacgcttcttcttctctggctacgataagaagcaaacgtggactgtccaaaataacgggcactcagtgatgatgttgctggagaacaaggccagcatttctggaggaggactgcctgccccataccaggccaaacagttgcacctgcactggtccgacttgccatataagggctcggagcacagcctcgatggggagcactttgccatggagatgcacatagtacatgagaaagagaaggggacatcgaggaatgtgaaagaggcccaggaccctgaagacgaaattgcggtgctggcctttctggtggaggctggaacccaggtgaacgagggcttccagccactggtggaggcactgtctaatatccccaaacctgagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcacgcagcctctctgttgcctcagctctccaagttccaggcttccggtccttagccttcccaggtgggactttaggcatgattaaaatatggacatatttttggag

SEQ ID NO: 13 - protein, 10 aa, Artificial Sequence
  Peptide corresponding to rat ZG16-p.
NSIQSRSSSY

SEQ ID NO: 14 - protein, 148 aa, Rattus norvegicus
MLAIALLVLLCASASANSIQSRSSSYSGEYGGKGGKRFSHSGNQLDGPITAIRIRVNRYYIIGLQVRYGTVWSDYVGGNRETEEIFLHPGESVIQVSGKYKSYVKQLIFVTDKGRYLPFGKDSGTSFNAVPLHPNTVLRFISGRSGSA

SEQ ID NO: 15 - DNA, 672 bp, Rattus norvegicus
aattcctcttagtccttctctgtgcctcggcatctgcaactactaggggaaagcctcaggatgttggccattgccctcttagtccttctctgtgcctcggcgtctgctaattccattcagtccaggtcctcctcttacagtggagagtatggcggtaaaggaggaaagcgattctctcactctggcaaccagctggacggccccatcactgccatccggatccgtgtcaacagatactacataataggtctccaggtgcgctacggcacagtgtggagtgactatgtgggtggcaacagggagactgaggagatctttctgcaccccggggaatctgtgatccaggtgtctggcaagtacaaatcttatgtgaagcagctgatcttcgtgacagacaaaggccgctacctgccttttggaaaagactcaggcacaagtttcaacgctgttcccttgcaccccaacactgtcctccgtttcattagtggccgatctggctctgcatagatgctatcagcctgcactgggatacctaccctagccactgcaacacttgttgaaacccaccatcctctgctgtggtgggtatgagaactcccttatcaacaagccccaggaaacatgcaaatagcttaataaaaggatatggttaaaaaaaaaaaaaaaaaaaaa

SEQ ID NO: 16 - protein, 47 aa, Homo sapiens
IALTLNGPKCLCGSDTAVQCELSPIPLSICLRKKVISCLILHTAFYT

SEQ ID NO: 17 - DNA, 370 bp, Homo sapiens
gattgcccttacactaaatggtcccaagtgtttatgtggttctgacactgccgtccagtgtgagttgtccccgataccattgtcaatatgcctgagaaaaaaagtaatttcctgccttattctacacacagcattttatacctagtctgagcgagaatctgagagtgatctttcctatagtatagagtagggtacatggatcttttcacaataagctgctgctcggaggcatttgtcacttctgagtttgcaactgtgtcagaggcccccagaaggctgcttccagtgaagtgaggtattaacactgaatagatttggatatactcctggtcacagtccgttcatcctgagccagaatcaaaaaaaaaaa

SEQ ID NO: 18 - protein, 394 aa, Rattus norvegicus
MEPILALLLALGPFQLSRGQSFQVNPPEPEVAVAMGTSLQINCSMSCDKDIARVHWHGLDTNLGNVQTLPGSRVLSVRGMLSDTGTRVCVGSCGSRSFQHSVKILVYAFPDQLEVTPEFLVPGRDQVVSCTAHNIWPAGPDSLSFALLRGEQSLEGAQALETEQEEEMQETEGTPLFQVTQRWLLPSLGTPALPALYCQVTMQLPKLVLTHRRKIPVLQSQTSPEPPSTTSAKPYILTSSHTTKAVSTGLSSVALPSTPLSSEGPCYPEIHQNPEADWELLCEASCGSGVTVHWTLAPGDLAAYHKREAGAQAWLSVLPLGPIPEGWFQCRMDPGGQVTSLYVTGQVIPNPSSMVALWIGSLVLGLLALAFLAYCLWKRYRPGPLPDSSSCTLL

SEQ ID NO: 19 - DNA, 1279 bp, Rattus norvegicus
gacagagaaaggcatggagcccatcctggccctcctgctggccctgggacccttccagctcagcagaggccagtccttccaggtgaatcctcctgagcctgaggtagctgtagccatgggcacatccctccagatcaactgcagcatgtcctgtgacaaggatatagcccgggtgcactggcatggcctggacaccaacctgggcaatgtgcagaccctcccaggcagcagggtcctctccgtacgaggcatgctgtcagacacgggcactcgtgtatgcgtgggttcctgtgggagtcgaagctttcagcactctgtgaagatccttgtgtatgcctttccagaccagctggaggtaaccccggagttccttgtacctggacgggaccaggtagtgtcctgcacagcccacaacatctggcctgcaggcccggacagtctttcctttgccttgctccgaggagagcagagcctggagggtgctcaggccctggaaacagagcaagaggaggagatgcaagagactgagggcactccactcttccaagtgacacaacgctggttgctgccctccctggggacccccgcccttcccgccctttattgccaggtcaccatgcagctgcccaaactggtgctgacacatagaaggaagattccagtcctacagagccagacctcaccagagccccccagcaccacctctgctaagccatacatcctgacctcatcacatactactaaggcagtctccactgggctcagcagtgtagccctgccttctacgcctctgagctccgaggggccctgctaccccgaaatccaccagaacccagaggcagactgggaacttctctgcgaagcctcctgtgggtctggagtgacggtgcattggaccctggctcctggtgacctggcagcctaccacaagagagaagctggggcccaggcatggctaagcgtgctgcccctgggccccattcctgagggctggttccagtgtcgcatggaccctggcgggcaggtgaccagtctgtatgttactggccaggtgatcccaaacccctcctccatggtcgccctgtggattggcagcttggtgctggggctgcttgcactcgccttccttgcctactgcctgtggaaacgctaccggccgggtcctctcccagactccagctcgtgtacgctcctatgaagttccattatgccagactaagggaggcaaaggattaccagatgtaggtttggggcatcaagatgatagtgtggccccttt

SEQ ID NO: 20 - protein, 381 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSPEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 21 - DNA, 1546 bp, Homo sapiens
gggactgagcatggatttcggactggccctcctgctggcggggcttctggggctcctcctcggccagtccctccaggtgaagcccctgcaggtggagcccccggagccggtggtggccgtggccttgggcgcctcgcgccagctcacctgccgcctggcctgcgcggaccgcggggcctcggtgcagtggcggggcctggacaccagcctgggcgcggtgcagtcggacacgggccgcagcgtcctcaccgtgcgcaacgcctcgctgtcggcggccgggacccgcgtgtgcgtgggctcctgcgggggccgcaccttccagcacaccgtgcagctccttgtgtacgccttcccggaccagctgaccgtctccccagcagccctggtgcctggtgacccggaggtggcctgtacggcccacaaagtcacgcccgtggaccccaacgcgctctccttctccctgctcgtcgggggccaggaactggagggggcgcaagccctgggcccggaggtgcaggaggaggaggaggagccccagggggacgaggacgtgctgttcagggtgacagagcgctggcggctgccgcccctggggacccctgtcccgcccgccctctactgccaggccacgatgaggctgcctggcttggagctcagccaccgccaggccatccccgtcctgcacagcccgacctccccggagcctcccgacaccacctccccggagtctcccgacaccacctccccggagtctcccgacaccacctccccggagcctcccgacaccacctccccggagcctcccgacaagacctccccggagcccgccccccagcagggctccacacacacccccaggagcccaggctccaccaggactcgccgccctgagatctcccaggctgggcccacgcagggagaagtgatcccaacaggctcgtccaaacctgcgggtgaccagctgcccgcggctctgtggaccagcagtgcggtgctgggactgctgctcctggccttgcccacgtatcacctctggaaacgctgccggcacctggctgaggacgacacccacccaccagcttctctgaggcttctgccccaggtgtcggcctgggctgggttaagggggaccggccaggtcgggatcagcccctcctgagtggccagcctttccccctgtgaaagcaaaatagcttggaccccttcaagttgagaactggtcagggcaaacctgcctcccattctactcaaagtcatccctctgttcacagagatggatgcatgttctgattgcctctttggagaagctcatcagaaactcaaaagaaggccactgtttgtctcacctacccatgacctgaagcccctccctgagtggtccccacctttctggacggaaccacgtactttttacatacattgattcatgtctcacgtctccctaaaaatgcgtaagaccaagctgtgccctgaccaccctgggcccctgtcgtcaggacctcctgaggctttggcaaataaacctcctaaaatgataaaaaaaaaa

SEQ ID NO: 22 - protein, 9 aa, Artificial Sequence
  Peptide corresponding to rat albumin.
TQKAPQVST

SEQ ID NO: 23 - protein, 173 aa, Artificial Sequence
  Fragment of rat albumin.
TQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 24 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAETEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 25 - protein, 608 aa, Homo sapiens; identical to SEQ ID NO: 24

SEQ ID NO: 26 - protein, 622 aa, Rattus norvegicus
IEFTDIIKQLSQNTYTPREAGSQKDENLAYYIENLFHDFKFSKVWRDEHYVKIQVKNSVSQNLVTINSGSNIDPVEAPEGYVAFSKAGEVTGKLVHANFGTKKDFEELNYSVNGSLVIVRAGKITFAEKVANAQSFNAIGVLIYMDRNTFPVVEADLQFFGHAHLGTGDPYTPGFPSFNHTQFPPSQSSGLPSIPVQTISRAPAEKLFKNMEGNCPPSWNIDSSCKLELSQNQNVKLTVNNVLKETRILNIFGVIKGYEEPDRYIVVGAQRDAWGPGVAKSSVGTGLLLKLAQVFSDMISKDGFRPSRSIIFASWTAGDYGAVGPTEWLEGYLSSLHLKAFTYINLDKVVLGTSNFKVSASPLLYTLMGKIMQDVKHPIDGKYLYRNSNWISKIEELSLDNAAFPFLAYSGIPAVSFCFCEDEDYPYLGTKLDTYEILIQKVPQLNQMVRTAAEVAGQFIIKLTHDIELTLDYEMYNSKLLSFMKDLNQFKADIKDMGLSLQWLYSARGDYFRATSRLTTDFHNAEKTNRFVMREINDRIMKVEYHFLSPYVSPRESPFRHIFWGSGSHTLSALVENLRLRQKNITAFNETLFRNQLALATWTIQGVANALSGDIWNIDNEF

SEQ ID NO: 27 - DNA, 3413 bp, Rattus norvegicus
catagagttcactgacatcatcaagcagctgagccagaatacatatactcctcgtgaggctggatctcagaaagacgagaatcttgcctattatattgaaaatctgttccatgactttaaattcagcaaagtctggcgagatgaacattatgtgaagattcaagtgaaaaacagtgtttctcaaaacttggtgaccataaattcaggtagtaacattgacccagtggaggctcctgagggttatgtggcatttagtaaagctggagaagttactggtaaactggtccatgctaattttggcactaaaaaggactttgaagaattaaattattctgtgaatggatctttagtgattgttagagcagggaaaattacttttgcagaaaaggttgcaaatgcccaaagctttaatgcaattggtgtcctcatctacatggacaggaatacgttccccgttgttgaggcagaccttcaattctttggacatgctcatctaggaactggggatccatatacacctggctttccttctttcaaccatactcagtttccgccatctcagtcatctggattgccttctatacctgtgcagacgatctcaagagctcctgcagaaaagctattcaaaaacatggaaggaaactgtcctcctagttggaatatagattcctcatgtaagctggaactttcacagaatcaaaatgtgaagctcactgtgaacaatgtactgaaagaaacaagaatacttaacatctttggcgttattaaaggctatgaggaaccagaccgctacattgtagtaggagcccagagagacgcttggggccctggtgttgcgaagtccagtgtgggaacaggtcttctgttgaaacttgcccaagtattctcagatatgatttcaaaagatggatttagacccagcaggagtattatctttgccagctggactgcaggagactatggagctgttggtccgactgagtggctggaggggtacctttcatctttgcatctaaaggctttcacttacattaatctggataaagtcgtcctgggtactagcaacttcaaggtttctgccagccccctattatatacacttatggggaagataatgcaggacgtaaagcatccgattgatggaaaatatctatatcgaaacagtaattggattagcaaaattgaggaactttccttggacaatgctgcattcccttttcttgcatattcaggaatcccagcagtttctttctgtttttgtgaggatgaggactatccttatttgggcactaaactagatacctatgagatattaattcagaaagttcctcagctcaaccaaatggttcgtacagcagcagaggtggccggtcagttcattattaaacttacccatgacattgagttgaccctggactatgagatgtacaacagcaaactactgtcatttatgaaggatctgaaccagttcaaagcagatataaaagatatgggtctaagtctacaatggctgtattctgctcgtggagactacttccgtgctacttctagactaacaactgattttcataatgctgagaaaacaaacagattcgtcatgagggaaatcaatgatcgtattatgaaagtggagtatcacttcctgtcaccctatgtatctccaagagagtctcctttccgacacatcttctggggctctggctctcacaccctctcagctttagtggagaacctgagacttcgtcagaaaaatatcactgctttcaatgaaacgctcttcagaaaccagttggccctggctacgtggactattcagggagttgcaaatgccctctctggtgacatttggaatattgacaatgagttttaaatgtaatgtgcataattaagtgagagagggtagtctgtttctagacttgagctggttgtgctaaattttcattagagctcgaattaatgttaaaaattctacccaatcatctaatgtgtttaggcagcagcttttagtgcagggttggacccacacttcaagttacagtggacaacactttccatgttcatgataccttcttagattatctttagaattttgagtcttttgtaataccttgctctttgcttcatggtcatgaaaatgtcagaaccagttgtaagaacattgctataagtcctgagggcactactagtatcttgaggtgggaggaagagggtgtatgtgaggggcagagtggtcgctgggtgtgattccccatctccatctgaccctcactgggattctccaattgagctgtatgcctgaaggatttagctggcttccattcccctaaagtagacagttacttttcagaagaggtgcaacttgttttcttgccagcaaggttgaactaggtccttctgctggataaaagaaaggaagtttgtctgtttacaggaataaggccttattggtttaacctttgtgttatttaggatgagaccagaagccaaagacttcaagttttctctccactgtcatctacccagtagtctttagttctttgggttgttttgtttttgttttgtttttcttttccaacactttctgaaaaagaacaggtttagactcagtctgtcagcagaacacactgccgagctcggtacccggggatcctctagagtcgacctgcaggcatgcaagctttccctatagtgagtcgtattagagcttggtgcatgcctgcaggtcgactctagaggatccccgggtaccgagctctggccaaagtgctggtcttcagggaagctctgtcgtttttggcactgagatatttattgtttatttatcagtgacagagttcactataaatagtgtttttttaatagaagataattatcggaagcagtgccttccataattatgacagttatactgtcgttttcttttaataaaagcagcatctgctaatgagacccacagatactggaagttttgcacttacggtcagcacttgcgggctttagaaaggagaaagccacaagccaaacaatatccgatgagctagaagaggattgggttaaataagagattcctagttgagttggaaaaaaatgataattctaagtccagtgagttgtggccaagttaaatgtcatttaaaggctatgatagtacatcaacaaaattctatagctcagtttattcaagatgtaactcaaatccaattttgcaaaatttccagtacctttgtcacaaacttaactcacattatcgggagcagtgtcttccataatgtataaagaacaaggtagtttttgcctaccacagtgtctatatcggagacagtgacctccatatgttacactaagggtgtacgtaattatcgggaacagtgtttcccataattttcttcatgcgatgacatcttcaaagcttg

SEQ ID NO: 28 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLYYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 29 - DNA, 5015 bp, Homo sapiens
cgggtggcggctcgggacggaggacgcgctagtgttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagggggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaataatatgtccttatcattgtataaaagctgttatgtgcaacagtgtggagattccttgtctgatttaataaaatacttaaacactgaaaaaaaaaaaaaaaaaaa

SEQ ID NO: 30 - protein, 161 aa, Rattus norvegicus
MNPVISITLLLSVLQMSRGQRVISLTACLVNQNLRLDCRHENNTNLPIQHEFSLTREKKKHVLSGTLGVPEHTYRSRVNLFSDRFIKVLTLANFTTKDEGDYMCELRVSGQNPTSSNKTINVIRDKLVKCGGISLLVQNTSWLLLLLLSLSFLQATDFISL

SEQ ID NO: 31 - DNA, 650 bp, Rattus norvegicus
ctgcaagctaggggagcccagacccaggacggagctattggcaccatgaacccagtcatcagcatcactctcctgctttcagtcttgcagatgtcccgaggacagagggtgatcagcctgacagcctgcctggtgaaccagaaccttcgactggactgccgtcatgagaataacaccaacttgcccatccagcatgagttcagcctgacccgagagaagaagaagcacgtgctgtcaggcaccctgggggttcccgagcacacttaccgctcccgcgtcaaccttttcagtgaccgctttatcaaggtccttactctagccaacttcaccaccaaggatgagggcgactacatgtgtgaacttcgagtctcgggccagaatcccacaagctccaataaaactatcaatgtgatcagagacaagctggtcaagtgtggtggcataagcctgctggttcaaaacacttcctggctgctgctgctcctgctttccctctccttcctccaagccacggacttcatttctctgtgactggttgggcccaaggagaaacaggaaacctcaaggtctgctgaagaggtcttgcttctcccggtcagctgactccctccccaagaccttcaaatatctcaaaacgcggggagaaatgg

SEQ ID NO: 32 - protein, 161 aa, Homo sapien
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 33 - DNA, 486 bp, Homo sapien
atgaacctggccatcagcatcgctctcctgctaacagtcttgcaggtctcccgagggcagaaggtgaccagcctaacggcctgcctagtggaccagagccttcgtctggactgccgccatgagaataccagcagttcacccatccagtacgagttcagcctgacccgtgagacaaagaagcacgtgctctttggcactgtgggggtgcctgagcacacataccgctcccgaaccaacttcaccagcaaatacaacatgaaggtcctctacttatccgccttcactagcaaggacgagggcacctacacgtgtgcactccaccactctggccattccccacccatctcctcccagaacgtcacagtgctcagagacaaactggtcaagtgtgagggcatcagcctgctggctcagaacacctcgtggctgctgctgctcctgctctccctctccctcctccaggccacggatttcatgtccctgtga